(19) United States
(12) Patent Application Publication
(10) Pub. No.: US 2008/0082154 A1
(43) Pub. Date: Apr. 3, 2008

(54) Stent Graft Delivery System for Accurate Deployment

(75) Inventors: David Yi Tseng, Princeton Junction, NJ (US); Francisco Moisef Llort, Skillman, NJ (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/864,071

(22) Filed: Sep. 28, 2007

Related U.S. Application Data
(60) Provisional application No. 60/848,197, filed on Sep. 28, 2006.
(60) Provisional application No. 60/848,198, filed on Sep. 28, 2006.
(60) Provisional application No. 60/848,232, filed on Sep. 28, 2006.
(60) Provisional application No. 60/848,246, filed on Sep. 28, 2006.

(51) Int. Cl. A61F 2/06

(52) U.S. Cl. 623/1.11

(57) ABSTRACT

A delivery system for deploying a stent graft at a lesion site is provided. The delivery system comprises a wire lumen and a support stent slidably positioned about the wire lumen. The support stent is expandable from a compressed delivery configuration to an expanded configuration. An inner sheath is retractably positioned about the support stent with the support stent in the delivery configuration. An anchor stent is slidably positioned about the inner sheath. A tubular graft proximal end is coupled to the anchor stent and deployable with the anchor stent from a compressed delivery configuration to a deployed configuration. An outer sheath is retractably positioned about the anchor stent and the graft in the compressed delivery configuration.

### RELATED APPLICATIONS

This application claims the benefit of: 1) provisional U.S. Patent Application Ser. No. 60/848,197, filed Sep. 28, 2006; 2) provisional U.S. Patent Application Ser. No. 60/848,198, filed Sep. 28, 2006; 3) provisional U.S. Patent Application Ser. No. 60/848,232, filed Sep. 28, 2006; and 4) provisional U.S. Patent Application Ser. No. 60/848,246, filed Sep. 28, 2006, all of which are incorporated herein by reference in their entirety.

### BACKGROUND OF THE INVENTION

Expandable endovascular prosthetic implants, such as stents and stent grafts, can be loaded into a catheter for delivery and deployment at a lesion site, such as an aneurysm or dissection within a patient's vascular system. The catheter is typically configured to retain the prosthetic implant in a delivery configuration during delivery to the lesion site. At the lesion site, the prosthetic implant may be deployed, for example by retracting a catheter sheath from the prosthetic implant's proximal end (nearest the patient's heart) to the distal end.

Prosthetic implants must be accurately placed to sufficiently cover the target lesion site during endovascular treatments or procedures. With many conventional catheters, implant movement during deployment may occur from frictional interference or contact with the catheter sheath as the catheter sheath is retracted from about the implant. Such implant movement may be an increased concern when implants having a high foreshortening percentage, such as a braided stent, are deployed. For example, during the deployment of a braided stent having a twenty percent foreshortening percentage, a proximal end and an opposing distal end of the stent may tend to converge, which causes the stent to migrate from a desired anchoring position within the target lesion site.

Moreover, covering undesired locations, such as healthy vessels and/or branch vessels, due to inaccurate implant placement may cause unfavorable clinical consequences, such as branch vessel occlusion and/or restenosis. Attempts to prevent or limit undesirable implant movement during deployment have included applying a lubricious coating to the conventional implant to reduce the frictional contact between the implant and the catheter sheath.

With thoracic stent graft placement, due to a high blood flow rate, a volume gradient, and/or a pressure gradient in the thoracic region, the proximal end of the stent graft may be pushed or moved distally as a result of blood flow and/or the pressure gradient within the thoracic region during initial deployment of the stent graft. Such migration may result in inaccurate positioning of the stent graft with respect to the lesion site. Further, in abdominal aneurysm procedures, an inadequate distance between an edge of the renal artery and an edge of the aneurysm, commonly referred to as a “short neck,” may prevent or limit a patient's acceptance of an endovascular treatment or procedure.

Also when a self-expanding stent graft is deployed within a curved portion of a blood vessel, desirably the stent graft will correspond to and/or accommodate the curvature of the blood vessel. Conventional stent grafts have included a plurality of discontinuous or noncontiguous stent elements that overlap each other to approximate the blood vessel curvature. Such element overlap in these stent grafts may result in angular deformity of the stent graft and/or an increased potential for structural damage to the stent graft and/or the blood vessel from repetitive pulsatile motion induced by blood flow and/or pressure variations.

Additionally, kinking or bending of a stent graft placed in a curved vessel may occur, which may compromise the blood flow through the stent graft. Attempts to provide stent grafts that are bent or otherwise curved to approximate the curvature of the blood vessel also may separate from the vessel wall because such stent grafts do not smoothly accommodate the curved vessel portion. This separation may lead to an attachment endoleak, a flap occlusion and/or portions of the stent graft projecting into the graft component of the stent graft and/or into the blood vessel wall, causing damage and/or injury.

### SUMMARY OF THE INVENTION

This invention relates to a stent graft delivery device. The present invention facilitates accurate positioning of a stent or stent graft at a desired lesion site while preventing or limiting undesirable stent or stent graft movement and/or migration. Further, a post-deployment placement of the stent or stent graft with respect to the lesion site can be accurately predicted or determined to prevent undesirable blockage or occlusion of branch vessels.

In one example, a delivery system for deploying a stent graft in a body vessel is provided. The delivery system comprises a wire lumen and a support stent slidably positioned about the wire lumen. The support stent includes a proximal end and a distal end, and is expandable from a compressed delivery configuration to an expanded configuration. An inner sheath is retractably positioned about the support stent with the support stent in the delivery configuration. An anchor stent is slidably positioned about the inner sheath. The anchor stent has a proximal end and a distal end, and is deployable from a compressed delivery configuration to a deployed configuration. A tubular graft having a proximal end and a distal end is also included. The graft proximal end is coupled to the anchor stent and deployable with the anchor stent from a compressed delivery configuration to a deployed configuration. An outer sheath is retractably positioned about the anchor stent and the graft in the compressed delivery configuration.

In another example, a delivery system for deploying a stent graft in a body vessel is provided. The delivery system includes a wire lumen slidably positionable about a guide wire. A support stent having a proximal end and a distal end is slidably positioned about the wire lumen and is expandable from a compressed delivery configuration to an expanded configuration. An inner sheath is retractably positioned about the support stent with the support stent in the compressed delivery configuration. An anchor stent having a proximal end and a distal end is slidably positioned about the inner sheath and deployable from a compressed delivery configuration to a deployed configuration. An outer sheath is retractably positioned about the anchor stent with the anchor stent in the insertion configuration. A handle is operatively coupled to each of the inner sheath and the outer sheath.

In a further example, a delivery system for deploying an endoluminal prosthesis within a body lumen at a target location is provided. The delivery system includes a delivery sheath having a proximal end and a distal end. The delivery sheath is configured to retain the prosthesis within the delivery system in an unexpanded configuration at the delivery sheath distal end. A support member having a proximal end and a distal end is positioned at least partially within the delivery sheath, where the proximal end of the support member is adjacent to the distal end of the prosthesis. A handle is also included. The handle is configured to impart relative movement to at least one of the delivery sheath and the support member.

In yet another example, a delivery system is provided. The delivery system includes a shaft defining a guide wire passage. A support member having a proximal end and a distal end is movably coupled to the shaft. The support member is configured to advance in a proximal direction along the shaft. A tubular delivery sheath is also included. The delivery sheath is configured to at least partially surround the support member and to retract in a distal direction along the shaft.

In another example, a delivery system for deploying a stent graft in a body vessel is provided. The delivery system includes a wire lumen and a support stent slidably positioned about the wire lumen. The support stent has a proximal end and a distal end, and is expandable from a compressed delivery configuration to an expanded configuration. An inner sheath is retractably positioned about the support stent with the support stent in the compressed delivery configuration. An anchor stent is slidably positioned about the inner sheath. The anchor stent has a proximal end and a distal end, and is deployable from a compressed insertion configuration to a deployed configuration. A tubular graft having a proximal end and a distal end is also included. The graft proximal end is coupled to the anchor stent and is deployable with the anchor stent from a compressed delivery configuration to a deployed configuration. An outer sheath is retractably positioned about the anchor stent and the graft in the compressed delivery configuration. A capture mechanism is operatively coupled to the proximal end of the anchor stent. The capture mechanism is initially configured to retain the proximal end of the stent in a delivery configuration. The capture mechanism is actuatable to release the proximal end of the anchor stent.

### BRIEF DESCRIPTION OF THE DRAWINGS

FIG. 1 is a side view of an exemplary stent graft in a deployed configuration in which a portion of the stent graft has a curvature of about 45°.

FIG. 2 is a side view of an exemplary stent graft in a deployed configuration in which a portion of the stent graft has a curvature of about 60°.

FIG. 3 is a side view of an exemplary stent graft in a deployed configuration in which a portion of the stent graft has a curvature of about 90°

FIG. 4 is a side view of an exemplary stent graft in a deployed configuration having an offset curvature of about 90°.

FIG. 5 is a side view of an exemplary stent graft in a deployed configuration in which a portion of the stent graft has a curvature of about 110°.

FIG. 6 is a side view of an exemplary stent graft in a deployed configuration in which a portion of the stent graft has a curvature of about 130°.

FIG. 7 is a perspective view of a proximal end of an exemplary stent graft on a delivery device including an anchor stent.

FIG. 8 is a side view of the proximal end of the stent graft shown in FIG. 7.

FIG. 9 is a perspective view of a distal end of an exemplary graft.

FIG. 10 is a side view of an exemplary stent in an arcuate initial configuration.

FIG. 11 is a side view of a partially deployed stent of FIG. 10.

FIG. 12 is an exploded perspective view of an exemplary stent graft delivery system.

FIG. 13 is a side view of the system shown in FIG. 12 in an initial delivery configuration.

FIG. 14 is a side view of the system shown in FIG. 12 with an outer sheath retracted.

FIG. 15 is a side view of the system shown in FIG. 12 with a deployed prosthesis.

FIG. 16 is a side view of the system shown in FIG. 12 with an inner sheath retracted.

FIG. 17 is an enlarged view of a portion of the system shown in FIG. 16.

FIG. 18 is a side view of the system shown in FIG. 12 in a final deployed configuration.

FIG. 19 is a schematic side view of a stent graft positioned with respect to a lesion site in a compressed delivery configuration.

FIG. 20 is a schematic side view of the stent graft shown in FIG. 19 with a distal end of the stent graft in a deployed configuration.

FIG. 21 is a schematic side view of the stent graft shown in FIG. 19 in a deployed configuration.

FIG. 22 is a schematic side view of a stent graft positioned with respect to a lesion site in a compressed delivery configuration.

FIG. 23 is a schematic side view of the stent graft shown in FIG. 22 with a distal end of the stent graft in a deployed configuration.

FIG. 24 is a schematic side view of the stent graft shown in FIG. 22 in a deployed configuration.

FIG. 25 is a front view of a portion of a capture mechanism.

FIG. 26 is a perspective view of a delivery device suitable for use with the stent graft shown in FIG. 22.

FIG. 27 is a perspective view of a capture mechanism suitable for use with the delivery device shown in FIG. 26.

FIG. 28 is a perspective view of a nose cone suitable for use with the delivery device shown in FIG. 26.

FIG. 29 is a side view of an exemplary delivery system illustrating movement of a retraction element.

FIG. 30 is a side view of the delivery system shown in FIG. 29 illustrating movement of a locking element.

FIG. 31 is a sectional view of the delivery system shown in FIG. 30 at sectional line A-A.

FIG. 32 is a side view of an exemplary delivery system illustrating movement of a first retraction element.

FIG. 33 is a side view of the delivery system shown in FIG. 32 illustrating movement of a second retraction element.

FIG. 34 is a sectional view of the delivery system shown in FIG. 32 at sectional line B-B.

FIG. 35 is a perspective view of an exemplary delivery system in an initial position.

FIG. 36 is a perspective view of the delivery system shown in FIG. 35 illustrating movement of a retraction element.

FIG. 37 is a perspective view of the delivery system shown in FIG. 35 illustrating movement of a second retraction element.

FIG. 38 is a perspective view of a portion of the delivery system shown in FIG. 35.

FIG. 39 is a sectional view of the portion of the delivery system shown in FIG. 38.

FIG. 40 is a perspective view of the delivery system shown in FIG. 35 with the housing removed.

FIG. 41 is another perspective view of the delivery system shown in FIG. 35 with the housing removed.

FIG. 42 is a sectional view of a portion of the delivery system shown in FIG. 38.

FIG. 43 is a perspective view of a portion of the delivery system shown in FIG. 3.

FIG. 44 is a perspective view of another portion of the delivery system shown in FIG. 38.

FIG. 45 is a perspective view of another portion of the delivery system shown in FIG. 38.

FIG. 46 is a perspective view of another portion of the delivery system shown in FIG. 38.

FIG. 47 is a side view of an exemplary delivery system illustrating movement of a retraction element.

FIG. 48 is a side view of the delivery system shown in FIG. 47 illustrating movement of a second retraction element.

FIG. 49 is a side view of the delivery system shown in FIG. 47 illustrating movement of a third retraction element.

FIG. 50 is a side view of an exemplary delivery system in an initial position.

FIG. 51 is a sectional view of the delivery system shown in FIG. 50.

FIG. 52 is a partial secondary side view of the delivery system shown in FIG. 50 with a retraction element drawn to an intermediate position.

FIG. 53 is a partial sectional side view of the delivery system shown in FIG. 50 with a retraction element drawn to a final position.

FIG. 54 is a side view of an exemplary delivery system in an initial position.

FIG. 55 is a sectional view of the delivery system shown in FIG. 54.

FIG. 56 is a partial sectional side view of the delivery system shown in FIG. 54 with an outer sheath retracted.

FIG. 57 is a partial sectional side view of the delivery system shown in FIG. 54 illustrating movement of the retraction element.

FIG. 58 is a perspective view of a portion of the delivery system shown in FIG. 54.

FIG. 59 is a partial sectional side view of an exemplary delivery system in an unlocked, initial position.

FIG. 60 is a partial sectional side view of the delivery system shown in FIG. 59 illustrating movement of an outer sheath retraction element.

FIG. 61 is a partial sectional side view of the delivery system shown in FIG. 59 illustrating movement of a graft retraction element.

FIG. 61A is an enlarged view of a portion of the system shown in FIG. 61.

FIG. 62 is a side view of the delivery system shown in FIG. 59 illustrating movement of an inner sheath retraction element.

FIG. 63 is a perspective view of an exemplary delivery system in an initial position.

FIG. 64 is a perspective view of the delivery system shown in FIG. 63 illustrating movement of an outer sheath retraction element.

FIG. 65 is a perspective view of the delivery system shown in FIG. 63 illustrating movement of an inner sheath retraction element.

FIG. 66 is a sectional view of a portion of the delivery system shown in FIG. 64.

FIG. 67 is a side view of a portion of the delivery system shown in FIG. 63 with the housing removed.

FIG. 68 is a side view of a portion of the delivery system shown in FIG. 65 with the housing removed.

FIG. 69 is a perspective view of a portion of the delivery system shown in FIG. 63 with a portion of the housing removed.

FIG. 70 is a side view of an exemplary delivery system in an initial position.

FIG. 71 is a side view of the delivery system shown in FIG. 70 illustrating movement of an outer sheath retraction element.

FIG. 72 is a side view of the delivery system shown in FIG. 70 illustrating movement of an inner sheath retraction element.

FIG. 73 is a perspective view of an exemplary delivery system in an initial position.

FIG. 74 is a perspective view of the delivery system shown in FIG. 73 illustrating movement of an outer sheath retraction element.

FIG. 75 is a perspective view of the delivery system shown in FIG. 73 illustrating movement of an inner sheath retraction element.

FIG. 76 is a side view of a portion of the delivery system shown in FIG. 73 with the housing removed.

FIG. 77 is a side view of a portion of the delivery system shown in FIG. 75 with the housing removed.

FIG. 78 is a partial sectional side view of a portion of the delivery system shown in FIG. 74.

FIG. 79 is a partial sectional side view of a portion of the delivery system shown in FIG. 74.

FIG. 80 is a perspective view of a portion of the delivery system shown in FIG. 73 with the housing removed.

FIG. 81 is a top view of an exemplary delivery system in an initial position.

FIG. 82 is a side view of the delivery system shown in FIG. 81.

FIG. 83 is a top view of the delivery system shown in FIG. 81 illustrating movement of an outer sheath retraction element.

FIG. 84 is a top view of the delivery system shown in FIG. 81 illustrating movement of an inner sheath retraction element.

FIG. 85 is a perspective view of an exemplary delivery system in an initial position.

FIG. 86 is a perspective view of the delivery system shown in FIG. 85 illustrating movement of an outer sheath retraction element.

FIG. 87 is a perspective view of the delivery system shown in FIG. 85 illustrating movement of an inner sheath retraction element.

FIG. 88 is a sectional view of a portion of the delivery system shown in FIG. 86.

FIG. 89 is a side view of a portion of the delivery system shown in FIG. 86 with the housing removed.

FIG. 90 is a side view of a portion of the delivery system shown in FIG. 87 with the housing removed.

FIG. 91 is a side view of an exemplary graft release mechanism.

FIG. 92 is a side view of an exemplary graft release mechanism.

FIG. 93 is a side view of the graft release mechanism shown in FIG. 92 with an outer sheath retracted.

FIG. 94 is a side view of an exemplary graft release mechanism.

FIG. 95 is a sectional side view of the graft release mechanism shown in FIG. 94.

FIG. 96 is a sectional side view of the graft release mechanism shown in FIG. 94 with a retaining ring retracted.

FIG. 97 is a side view of an exemplary graft release mechanism.

FIG. 98 is a sectional side view of the graft release mechanism shown in FIG. 97 with a retaining ring retracted.

FIG. 99 is a sectional side view of the graft release mechanism shown in FIG. 98 with a graft in a delivery configuration.

FIG. 100 is a sectional side view of the anchor stent release mechanism shown in FIG. 98 with a graft in a deployed configuration.

FIG. 101 is a side view of an exemplary graft release mechanism.

FIG. 102 is a side view of the anchor stent release mechanism shown in FIG. 101 with a graft partially deployed.

FIG. 103 is a side view of the graft release mechanism shown in FIG. 101 with a graft partially deployed.

FIG. 104 is a sectional side view of an exemplary support member advancement mechanism.

FIG. 105 is a sectional side view of the support member advancement mechanism shown in FIG. 104.

FIG. 106 is a sectional side view of an exemplary support member advancement mechanism in an initial position.

FIG. 107 is a sectional side view of the support member advancement mechanism shown in FIG. 106 in a final position.

FIG. 108 is a sectional side view of an exemplary support member advancement mechanism in an initial position.

FIG. 109 is a sectional side view of the support member advancement mechanism shown in FIG. 108 in a final position.

FIG. 110 is a sectional side view of an exemplary support member advancement mechanism in an initial position.

FIG. 111 is a sectional side view of a portion of the support member advancement mechanism shown in FIG. 110.

FIG. 112 is a sectional side view of an exemplary support member advancement mechanism in an initial position.

FIG. 113 is a sectional side view of a portion of the support member advancement mechanism shown in FIG. 112.

FIG. 114 is a partial sectional view of an exemplary prosthesis delivery system.

FIG. 115 is a partial sectional view of an exemplary prosthesis delivery system before deployment.

FIG. 116 is a partial sectional view of an exemplary prosthesis delivery system during deployment.

FIG. 117 is a partial sectional view of an exemplary prosthesis delivery system after deployment.

### DETAILED DESCRIPTION OF THE INVENTION

The present invention provides a delivery system for deploying a stent graft in a body vessel, for example for repairing and/or treating aneurysms such as abdominal aortic and thoracic aortic aneurysms. The stent and stent graft may have a configuration that, upon deployment, adapts or conforms to the body vessel. More specifically, with the stent or stent graft positioned at a lesion site within a curved portion of a blood vessel, the stent or stent graft is adaptable to the anatomical curvature of the blood vessel.

The present invention facilitates accurate positioning of the stent or stent graft at the desired lesion site while preventing or limiting undesirable stent or stent graft movement and/or migration. Further, a post-deployment placement of the stent or stent graft with respect to the lesion site can be accurately predicted or determined to prevent undesirable blockage or occlusion of branch vessels.

The stent graft may be deployed from a distal end (related to a position of a patient's heart) to the proximal end of the stent graft. The distal end is commonly referred to as the “bottom” position and the proximal end is commonly referred to as the “up” position. By deploying the stent graft in a “bottom-up” procedure, a distal end of the stent graft is precisely and accurately positioned at the desired lesion site and a post-deployment placement of the stent graft with respect to the lesion site can be accurately predicted or determined to prevent undesirable blockage or occlusion of branch vessels.

The present invention is described below in reference to its application in connection with endovascular treatment of thoracic aortic aneurysms and dissections. However, it is likewise applicable to any suitable endovascular treatment or procedure including, without limitation, endovascular treatment of abdominal aortic aneurysms and dissections.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

### DEFINITIONS

“Adaptable” refers to the ability of the stent graft components to move and/or adjust to the curvature of the blood vessel

References to “endovascular” are to be understood to refer to within blood vessels.

“Body vessel” means any tube-shaped body passage lumen that conducts fluid, including but not limited to blood vessels such as those of the human vasculature system, esophageal, intestinal, biliary, urethral and ureteral passages.

“Implantable” refers to an ability of a prosthetic implant to be positioned, for any duration of time, at a location within a body, such as within a body vessel. Furthermore, the terms “implantation” and “implanted” refer to the positioning, for any duration of time, of a prosthetic implant at a location within a body, such as within a body vessel.

“Biocompatible” refers to a material that is substantially non-toxic in the in vivo environment of its intended use, and that is not substantially rejected by the patient's physiological system (i.e., is non-antigenic). This can be gauged by the ability of a material to pass the biocompatibility tests set forth in International Standards Organization (ISO) Standard No. 10993 and/or the U.S. Pharmacopeia (USP) 23 and/or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity and/or immunogenicity. A biocompatible structure or material, when introduced into a majority of patients, will not cause a significantly adverse, long-lived or escalating biological reaction or response, and is distinguished from a mild, transient inflammation which typically accompanies surgery or implantation of foreign objects into a living organism.

The term “string” refers to any continuous strand of material. For example, strings may include, but are not limited to, monofilaments, filaments, fibers, yarns, cords, strings, threads, and sutures.

The term “retraction element” refers to any element able to impart motion to another element. For example, retraction elements may include, but are not limited to, knobs, rotary knobs, levers, grips, slides, handles, shafts, arms, tabs, cranks, slides, pivots, and stems.

The term “locking element” refers to any element able to limit or otherwise prevent movement of another element. For example, locking elements may include, but are not limited to, knobs, levers, grips, handles, shafts, arms, cranks, pins, tabs, buttons, poles, pivots, rods, stems, and lockouts.

Stent and Stent Graft

Stents and stent grafts according to the present invention may have a configuration upon deployment during an endovascular procedure permitting adaptation of the stent, graft or stent graft to the anatomical configuration of the blood vessel. For example, they may have a curved configuration upon deployment during an endovascular procedure, permitting adaptation of the stent, graft or stent graft to the anatomical curvature of the blood vessel. In one example, the configuration may be provided by a shape memory of the stent as a result of a secondary annealing process, as described in greater detail below.

At the lesion site, a stent may be movable between a compressed and/or deformed delivery configuration and a deployed configuration to adjust to the configuration of a blood vessel. The stent may be formed or fabricated in an initial configuration having a curvature of about 0° to about 180°. In one example, the stent may have a curvature of about 180° in the initial configuration. In a deployed configuration, the stent is adaptable to approximate the configuration, such as a curvature, of the blood vessel portion or lesion site within which the stent is positioned. The curvature of the stent in the deployed configuration may be different than the curvature in the initial configuration.

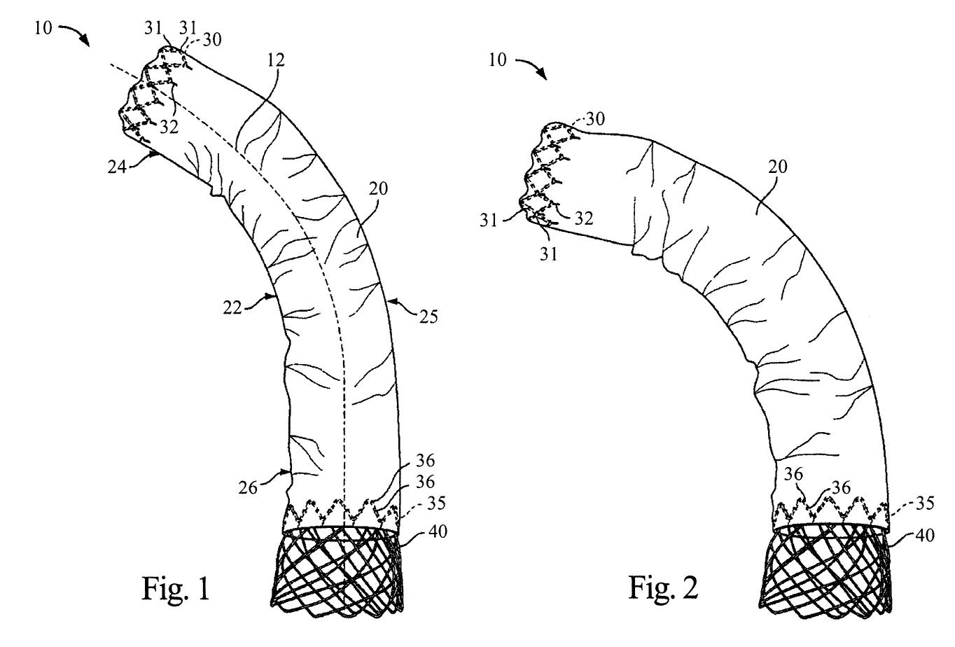

FIGS. 1-6 illustrate exemplary stent grafts. Stent graft 10 may be positioned within a blood vessel, such as a patient's aorta, to reinforce a weak spot or lesion site in the blood vessel at or near an aneurysm. In one example, stent graft 10 is positioned within the blood vessel at a curved portion of the blood vessel, such as at the aortic arch. Stent graft 10 provides strength to the injured or diseased blood vessel at the aneurysm and allows blood to flow through stent graft 10 without further stress and/or trauma to the aneurysm, thus, preventing enlargement and/or rupture of the blood vessel at the lesion site.

In one example, the stent graft 10 includes a braided stent, as described in greater detail below. A braided stent facilitates smoothly approximating a curvature of the blood vessel without introducing additional stress points at the vessel wall at or near the lesion site. Forming the braided stent by a suitable annealing or heat treating process to an arcuate initial configuration, material straightening stresses on the blood vessel wall may be eliminated or reduced. Thus, this further reduces stresses applied by the support stent and/or stent graft against the vessel wall.

Stent graft 10 defines a longitudinal axis 12 along a length of stent graft 10, as shown in FIG. 1. Stent graft 10 may have any suitable length corresponding to a length of the lesion site at which the stent graft is to be positioned. Stent graft 10 may be anchored tightly to an interior wall surface of the blood vessel proximally and/or distally to the lesion site.

FIGS. 1-6 show an exemplary stent graft 10 in an arcuate deployed configuration having a curvature of about 0° to about 180°. In one example, in the deployed configuration, stent graft 10 has a configuration substantially similar to the configuration of stent graft 10 in the initial configuration. In another example, in the deployed configuration, stent graft 10 has a curvature different than the curvature of stent graft 10 in the initial configuration. FIGS. 1-6 illustrate stent graft 10 in various deployed configurations having a curvature of about 45°, as shown in FIG. 1, to about 130°, as shown in FIG. 6.

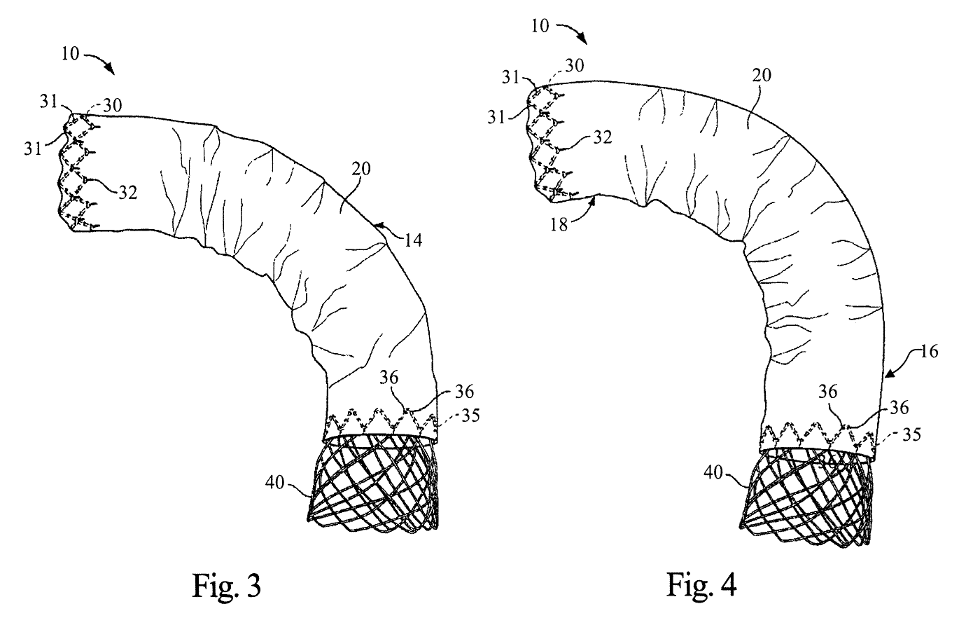

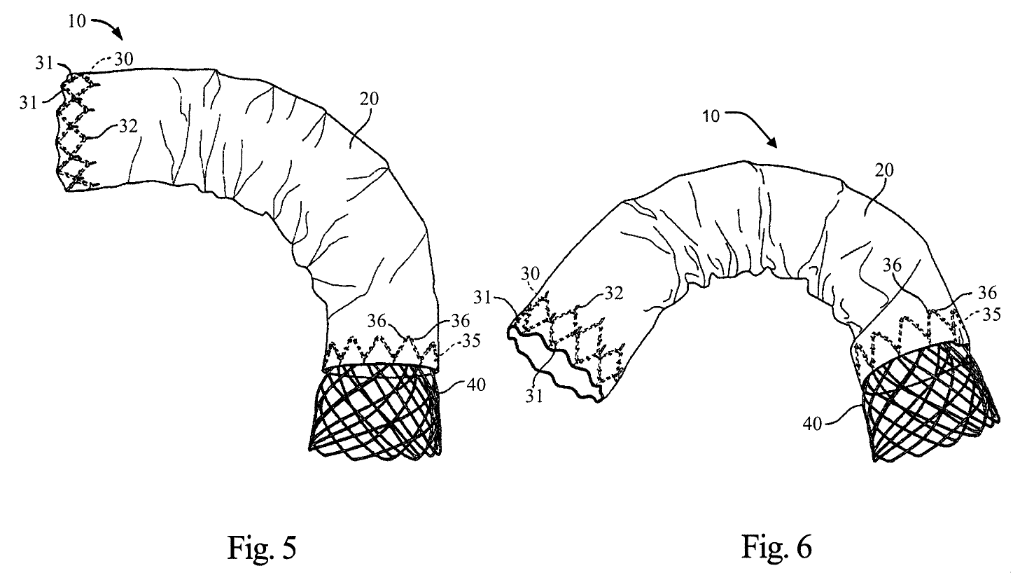

In the deployed configuration stent graft 10 may have a curvature of about 45° as shown in FIG. 1, about 60° as shown in FIG. 2, about 90° as shown in FIGS. 3 and 4, about 110° as shown in FIG. 5 or about 130° as shown in FIG. 6. An arcuate or curved portion of stent graft 10 may be positioned at a center portion 14 of stent graft 10 as shown in FIG. 3, at or near a proximal portion 18 of stent graft 10 as shown in FIG. 4 or at or near a distal portion 16 of stent graft 10 (not shown).

An external diameter of distal portion 16 of stent graft 10 may be different than an external diameter of proximal portion 18 of stent graft 10. The external diameter of distal portion 16 may correspond to an internal diameter of the blood vessel at or near a distal end of the curved blood vessel portion and the external diameter of proximal portion 18 may correspond to an internal diameter of the blood vessel at or near a proximate end of the curved blood vessel portion. In one example, the external diameter of proximal portion 18 is greater than the external diameter of distal portion 16.

Graft

As shown in FIGS. 1-6, stent graft 10 may include a graft 20 formed of a suitable biocompatible material. Graft 20 may include any suitable biocompatible synthetic and/or biological material, which is suitable for facilitating repair to the injured or diseased blood vessel.

Graft 20 has a body 22 that defines a proximal end 24, a midsection 25 and an opposing distal end 26. In one example, body 22 has a tubular configuration and is flexible to adapt to contact an inner surface of the curved blood vessel portion. Graft 20 may be fabricated from a suitable fabric or cloth material that is flexible to contact an inner surface of the curved blood vessel portion and/or adjust to the curvature of the inner surface. Referring to FIG. 1, proximal end 24 is configured, upon deployment of graft 20 at the lesion site, to contact and/or sealingly anchor to the interior wall surface of the vessel at a proximal anchoring location. Similarly, distal end 26 is configured to contact and/or sealingly anchor to the interior wall surface at a distal anchoring location.

The stent graft 10, including graft 20, may be delivered to the lesion site using a suitable delivery device, such as a catheter, that is configured to retain stent graft 10 in a compressed delivery configuration as stent graft 10 is delivered through the patient's vascular system to the lesion site. At the lesion site, stent graft 10 may be partially deployed. More specifically, graft 20 may be positioned at the lesion site such that proximal end 24 is positioned proximally with respect to the lesion site. With proximal end 24 sealingly anchored to the interior wall surface, distal end 26 may contact and/or sealingly anchor to the interior wall surface of the vessel at the distal anchoring location positioned distal with respect to the lesion site. In another example, graft 20 is positioned at the lesion site such that distal end 26 contacts or anchors to the interior wall surface distal to the lesion site. With distal end 26 contacting the interior wall surface, proximal end 24 sealingly anchors to the interior wall surface of the vessel at the proximal anchoring location positioned proximal with respect to the lesion site.

Anchor Stent

As shown in FIGS. 1-6, an anchor stent 30 may be coupled to graft 20 using a suitable coupling mechanism, such as a string or stitching 31. Referring to 1-8, anchor stent 30 may be coupled to an inner surface of graft 20 at proximal end 24. Anchor stent 30 may include at least one projection, such as a plurality of barbs 32, which extend through graft 20 and outwardly with respect to an outer surface of graft 20. Barbs 32 may be integrally formed with anchor stent 30. Barbs 32 are configured to penetrate and/or imbed into a blood vessel wall, such as the aortic wall, with stent graft 10 in the deployed configuration for facilitating retaining stent graft 10 accurately and properly positioned at the lesion site. In one example, anchor stent 30 expands radially outwardly with respect to graft 20 such that barbs 32 penetrate and/or imbed into the blood vessel wall.

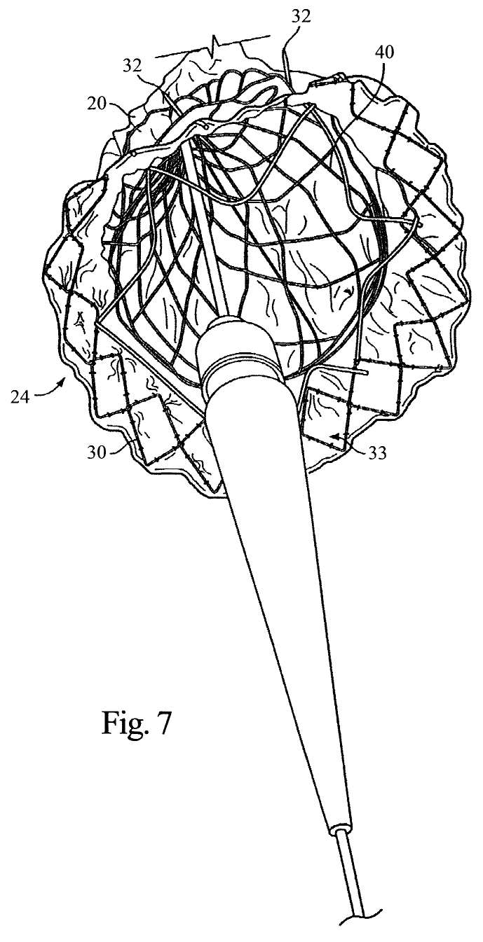

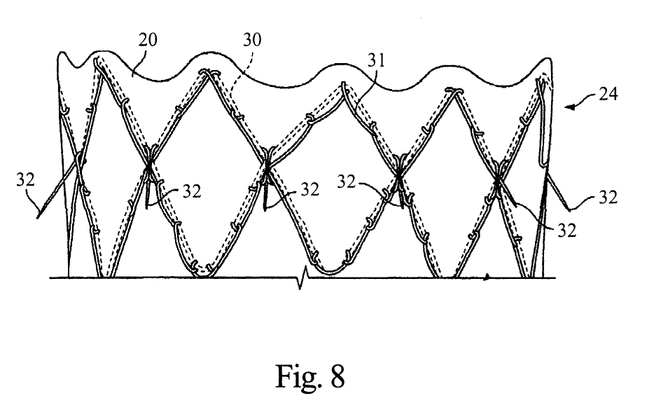

As shown in FIG. 7, anchor stent 30 may be configured to form a plurality of diamond shaped voids 33. Anchor stent 30, including integrally formed barbs 32, may be fabricated using a suitable laser cutting process, or other suitable process. The anchor stent may also comprise a Z stent or other type of stent.

Locking Ring

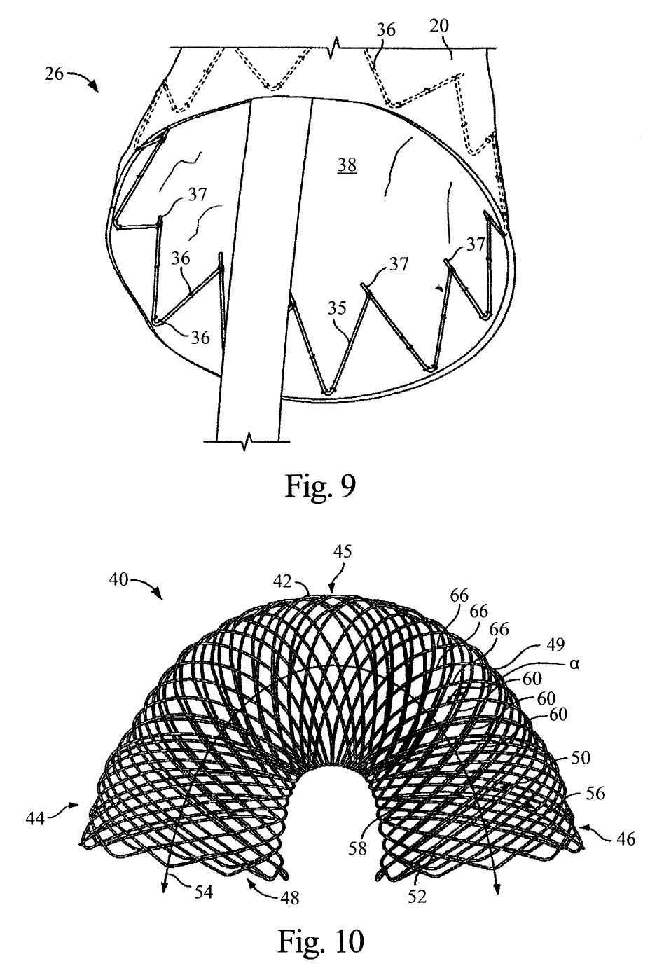

A locking ring 35 also may be coupled to graft 20 at distal end 26. As shown in FIG. 9, locking ring 35 is coupled to distal end 26 using a suitable coupling mechanism, such as a string or stitching 36. The locking ring 35 may include at least one projection, such as a plurality of prongs 37, which extend inwardly from locking ring 35 into a passage 38 defined by graft 20. The prongs 37 may be integrally formed with locking ring 35. Prongs 37 may be configured to interfere with and/or couple to a support stent 40 positioned within graft 20 for facilitating maintaining support stent 40 accurately positioned within graft 20. The prongs 37 may be relatively short and blunt as opposed to barbs 32, which are relatively longer and sharp or pointed. Further components or mechanisms may be incorporated into locking ring 35 that may be configured to interfere with and/or couple to support stent 40 to maintain support stent 40 accurately positioned within graft 20 without undesirably interfering with blood flow through passage 38.

Locking ring 35, with may include integrally formed prongs 37, may be fabricated using a suitable laser cutting process. However, locking ring 35 also may comprise a Z stent or other type of stent.

Support Stent

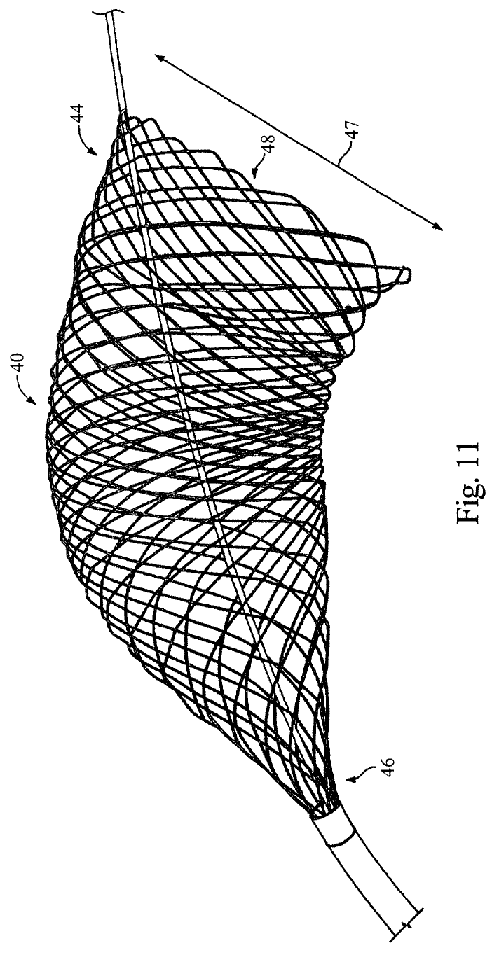

Referring further to FIGS. 1-6, stent graft 10 includes support stent 40 positionable within graft 20 and coupled to graft proximal end 24 and/or graft distal end 26. FIG. 10 shows support stent 40 in an arcuate initial configuration. FIG. 11 shows support stent 40 in a delivery configuration and partially deployed, as described in greater detail below. Support stent 40 may be fabricated from one or more shape memory wires. For example, support stent 40 may be formed from one or more of braided nitinol wires. In one example, support stent 40 is fabricated from a continuous braided nitinol wire, as described below. Support stent 40 also may be formed of a suitable biocompatible material including, without limitation, a suitable metal, such as stainless steel, platinum and/or titanium, alloy and/or composite material having suitable elastic properties.

At least a portion of support stent 40 may be made of a polymeric material having suitable strength, such as polyetheretherketon (PEEK), polyethersulfon (PES) or polyimide (PI). Support stent 40 also may include any suitable biocompatible synthetic and/or biological material, which is suitable for repair of the injured or diseased blood vessel. Support stent 40 may be fabricated by annealing a straight stent into an arcuate configuration, laser cutting a bent or curved tube to form a continuous laser cut arcuate stent or casting a polymeric material to form a polymer cast arcuate stent.

Support stent 40 has a body 42 that defines a proximal end 44, a midsection 45 and an opposing distal end 46. An external diameter of proximal end 44 and/or an external diameter of distal end 46 may be greater than an external diameter of midsection 45. Further, the external diameter of proximal end 44 may be similar to or different from the external diameter of distal end 46. In one example, body 42 has a tubular configuration and is expandable in a radial direction, as represented by directional arrow 47 in FIG. 11, with respect to a longitudinal axis of support stent 40 that corresponds to longitudinal axis 12.

Support stent 40 may be positioned within graft 20. For example the proximal end 44 of the support stent 40 may be attached at or near the proximal end 24 of the graft 20. For example, proximal end 24 may be sewed, stitched, glued or otherwise attached to the graft 20. In its compressed delivery configuration, only the proximal end 44 of the support stent 40 is attached to the graft. In this configuration, the distal end 46 of the support stent 40 defines a freely movable end portion of support stent 40, i.e., support stent distal end 46 is not directly coupled or attached to graft 20.

In one example, anchor stent 30 expands radially outwardly with respect to graft 20 such that barbs 32 penetrate and/or imbed into the blood vessel wall. With support stent proximal end 44 coupled to graft proximal end 24, support stent distal end 46 may define a freely movable end portion of support stent 40, e.g., support stent distal end 46 is not directly coupled or attached to graft 20. In one example, with stent graft proximal end 18 coupled to the blood vessel wall, support stent distal end 46 may be deployed. In an alternative example, stent graft distal end 16 is deployed. With stent graft distal end 16 contacting the blood vessel wall, stent graft proximal end 18 may be deployed.

Support stent distal end 46 is expandable to contact an inner surface of graft 20 and engage the graft 20 at or near the distal end 26 of graft 20. An engaging mechanism, such as locking ring 35, provided at or near the distal end 26 of graft 20, may engage the support stent 40 at or near the distal end 46 of support stent 40. In one example, the engaging mechanism may include prongs 37 extending radially inward from locking ring 35. Prongs 37 provided on the locking ring 35 may engage or interfere with the support stent distal end 46. In this manner, the support stent 40 may accurately positioned within graft 20. In the deployed configuration, support stent 40 and graft 20 define a passage 48 through which blood flows, as shown in FIGS. 1-6.

Support stent 40 has a suitable length extending between support stent proximal end 44 and support stent distal end 46 and along the length of graft 20. The length of support stent body 42 may be greater than or equal to the length of graft body 22. In one example, the length of support stent body 42 may be at least 1 cm greater than the length of graft body 22. For example, support stent distal end 46 extends at least 1 cm in a distal direction along longitudinal axis 12 beyond graft distal end 26. Support stent 40 may be extendable over a variable range of lengths beyond graft distal end 26, as required by certain applications to cover a dissected portion of the aorta. Such length may approach at least about 30 cm in certain applications.

As described above, support stent 40 may be a braided stent. As shown in FIG. 10, braided stent 40 may have an arcuate initial configuration, which may be configured to correspond to a curvature of the blood vessel. As shown in FIG. 10, braided stent 40 may include a continuous structural wire 49 forming a first helical wire portion 50 having a first translational direction, as shown by direction arrow 52, about an axis 54 of stent 40. Structural wire 49 further forms a second helical wire portion 56 having a second translational direction, as shown by direction arrow 58 in FIG. 10, about axis 54 opposite the first translational direction and interwound with first helical wire portion 50. First helical wire portion 50 and second helical wire portion 56 may form a double helix where first helical wire portion 50 and second helical wire portion 56 are congruent helices with a same axis, namely axis 54. Further, first helical wire portion 50 may intersect and/or be wound with second helical wire portion 56 at a braiding angle α as shown in FIG. 10. For example, braiding angle α is at least about 120°.

Alternatively, braided stent 40 may include multiple wires. For example, braided stent 10 may include a first helical wire having a first translational direction, as shown by direction arrow 52 in FIG. 10, about axis 54 of stent 40 and a second helical wire having a second translational direction, as shown by direction arrow 58 in FIG. 10, about axis 54 opposite the first translational direction and interwound with the first helical wire. The first helical wire and the second helical wire may form a double helix wherein the first helical wire and the second helical wire are congruent helixes with a same axis, namely axis 54.

As shown in FIGS. 10 and 11, first helical wire portion 50 generally includes a plurality of coil segments or windings 60. Additionally, second helical wire portion 56 includes a plurality of coil segments or windings 66. Each coil winding 60 is movable with respect to adjacent coil windings 60 and/or each coil winding 66 is movable with respect to adjacent coil windings 66 to contact and form or adjust to an inner surface of a corresponding curved portion of the blood vessel. First helical wire portion 50 and second helical wire portion 56 may have an equal number of coil windings 60 and 66, respectively, such that in the deployed configuration, braided stent 40 smoothly approximates the curvature of the interior wall of the blood vessel.

Support stent 40 may be movable from the initial configuration to the deployed configuration to correspond with the curvature of the interior wall of the blood vessel, while eliminating or limiting individual stress points or areas exerted by support stent 40 on the interior wall of the blood vessel. When support stent 40 has an arcuate initial configuration, support stent 40 does not exert undesirable forces against the interior vessel wall while positioned at the lesion site within the curved portion.

Support stent 40 may be heat-treated to form support stent 40 in the arcuate initial configuration. Support stent 40 also may include an annealed material. Support stent 40 may be annealed to form support stent 40 in the arcuate initial configuration. For example, support stent 40 may be fabricated by forming continuous structural wire 49 into first helical wire portion 50 and second helical wire portion 56. The formed support stent 40 is then annealed to move and retain the stent at the arcuate initial configuration. In this example, axis 54 defines a curvature of support stent 40. During the annealing process, the material is exposed to an elevated temperature for an extended period of time and then slowly cooled. The microstructure of the material is changed as the material is heated and then slowly cooled to alter the mechanical properties of the material. The annealing process further negates any internal stresses developed within the material during the machining and/or casting processes

Body 42 of support stent 40 may have a differential compliance, i.e., a compliance that varies along a length of body 42, for facilitating adjusting to a curvature of the blood vessel at the lesion site. For example, proximal end 44 may have a “soft” compliance or stiffness that at least approaches or approximates the physiological compliance of the blood vessel for facilitating positioning support stent 40 within a curved or angular portion of the blood vessel. The stiffness of proximal end 44 may approach or approximate the stiffness of the blood vessel to prevent or limit erosion of the blood vessel due to a radial force exerted by support stent 40 against the interior wall of the blood vessel with support stent 40 deployed. Here, distal end 46 has a greater stiffness than the stiffness of proximal end 44.

A heat treatment process may be used to facilitate adjusting a radial strength of at least a portion of body 42 to produce support stent 40 having differential compliance. Proximal end 44 may be made of a softer material than a material used to make body 42 including distal end 46. Suitable materials include, without limitation, a metal material, an alloy material, such as a nitinol material, or a polymeric material. In this example, proximal end 44 is made of a material having a stiffness that complies with a stiffness of the blood vessel and distal end 46 is made of a material having a greater stiffness than the stiffness of proximal end 44. Distal end 46 may be made of a material having a stiffness less than a stiffness of proximal end 44.

As shown in FIGS. 1-6, the stent graft may include a support stent, having a proximal and distal end, that is at least partially disposed within a tubular graft material. The graft material may have an anchor stent positioned at or near either or both the proximal and distal end of the graft. As shown in FIGS. 1-6, an anchor stent may be attached to the graft proximal end. The proximal end of the support stent may be attached to the graft at or near the proximal end of the graft. The graft also may include a locking mechanism such as a locking ring at or near the distal end of the graft. The locking mechanism, during expansion of the support stent, may engage the support stent at or near the distal end of the support stent. When the support stent, for example is a braided stent, the support stent in its compressed delivery configuration may have a length greater than the support stent in the expanded delivery configuration. Because the length of the support stent may decrease upon expansion, the support stent is attached to the graft only at or near the proximal end of the graft in the delivery configuration. During expansion of the support stent, the locking mechanism engages the support stent in the deployed configuration to thereby substantially hold or fix the diameter and length of the support stent in the deployed configuration.

Delivery System

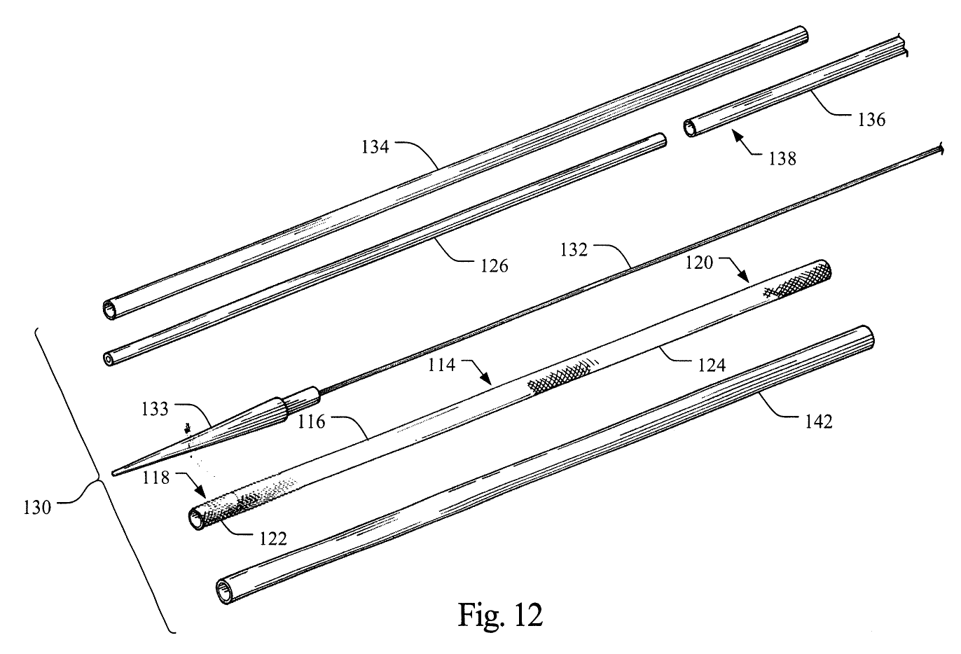

FIGS. 12-18 show a delivery system for delivering and/or deploying a prosthetic implant, such as a stent or a stent graft, at a lesion site during a thoracic aortic aneurysm repair procedure. During a thoracic aortic aneurysm repair procedure, a delivery system 130 I used to deliver and/or position a stent graft, for example stent graft 110, with respect to the lesion site at or near the aneurysm. Delivery system 130 may include a wire lumen 132 slidably positionable about a guide wire (not shown) initially positioned within a vessel of a patient. In one example, the guide wire is advanced by the surgeon through the vessel from the patient's femoral artery and positioned within the aorta. Wire lumen 132 defines a passage (not shown) therethrough such that wire lumen 132 is slidably positioned about the guide wire. In one example, a nose cone 133 is coupled to or integrated with wire lumen 132 for facilitating advancing the stent graft to the lesion site.

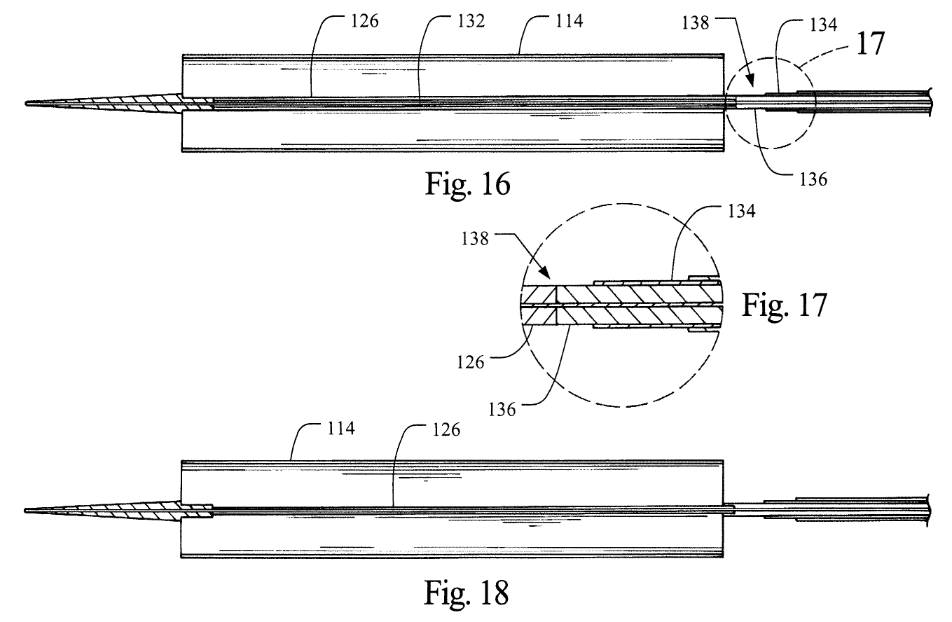

Referring further to FIGS. 16 and 17, support stent 126 may be slidably positioned about wire lumen 132. An inner sheath 134 is retractably positioned about support stent 126 with support stent 126 in the compressed delivery configuration. Inner sheath 134 is positioned about at least a portion of support stent 126 to maintain support stent 126 in the compressed delivery configuration as stent graft 110 is advanced to the lesion site. With stent graft 110 positioned within the vessel as desired, inner sheath 134 is retractable for facilitating deployment of support stent 126 from the compressed delivery configuration to the expanded deployed configuration, as described in greater detail below.

Delivery system 130 also may include a support member 136 slidably positioned about wire lumen 132. Support member 136 defines a proximal end 138 and an opposing distal end 140. Proximal end 138 contacts a distal end of support stent 126 with support stent 126 in the compressed delivery configuration, as shown in FIGS. 16 and 17.

In one example, support member 136 maintains a substantially constant force against support stent 126 as inner sheath 134 is retracted from about support stent 126 to prevent or limit undesirable movement of support stent 126 in the distal direction and retain support stent 126 properly positioned at the lesion site. In another example, support stent 126 expands as inner sheath 134 is retracted with respect to support stent 126. In various examples, inner sheath 134 and support member 136 move in opposite directions to facilitate minimizing a foreshortening of support stent 126, such as a braided stent. A ratio of opposing movement may be about 1:1 to about 1:3.

As shown in FIG. 12, graft 114 is slidably positioned about inner sheath 134. In one example, graft 114 may include anchor stent 30, and locking ring 35, as described above. An outer sheath 142 is retractably positioned about graft 114 with graft 114 in the delivery configuration. Outer sheath 142 is positioned about at least a portion of graft 114 to maintain graft 114 in the delivery configuration as stent graft 110 is advanced to the lesion site. With stent graft 110 positioned within the vessel as desired, outer sheath 142 is retractable for facilitating deployment of graft 114 from the delivery configuration to the deployed configuration, as described in greater detail below.

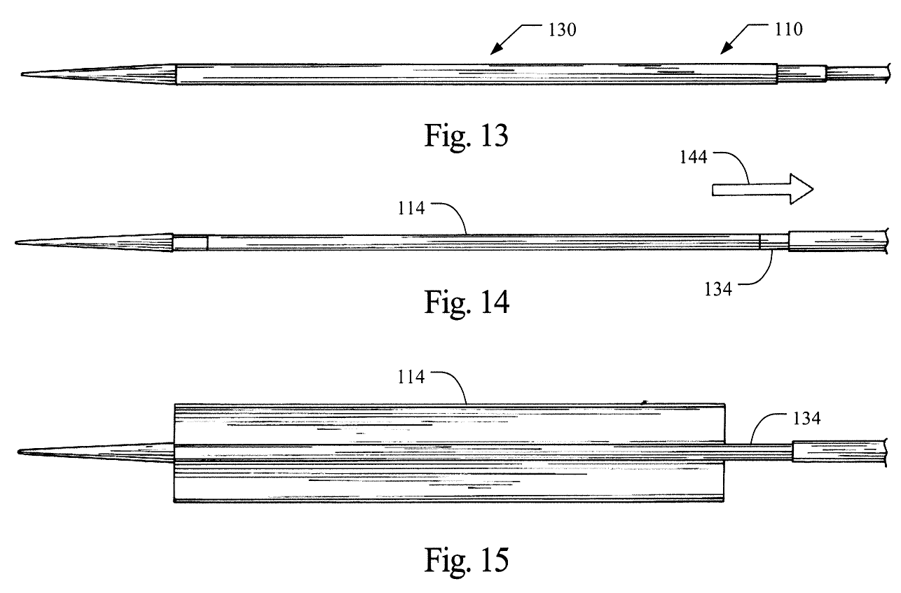

Referring to FIGS. 12-18, during a thoracic aortic aneurysm repair procedure, stent graft 110 is delivered to and deployed at the lesion site. A guide wire is inserted through a patient's vasculature structure. With stent graft 110 positioned within delivery system 130 as shown in FIG. 13, delivery system 130 is advanced to the lesion site along the guide wire. Delivery system 130 is positioned about the guide wire through the passage defined by lumen 132 with nose cone 133 at a leading end of delivery system 130.

With delivery system 130 at the lesion site, outer sheath 142 is moved in a distal direction, as shown by directional arrow 144 in FIG. 14, to retract outer sheath 142 and expose at least a portion of graft 114. As shown in FIG. 15, graft 114 is deployed at the lesion site. Graft 114 expands in a radial direction with respect to lumen 132 between the delivery configuration and the deployed configuration. In the deployed configuration, an outer radial surface of graft 114 contacts the interior surface of the vessel wall at the lesion site and graft 114 defines a passage therethrough. Proximal end 118 of graft 114 is positioned proximal to the aneurysm and distal end 120 is positioned distal to the aneurysm. As described above graft 114 may include anchor stent 30 and locking ring 35. Anchor stent 30 is positioned proximal to the aneurysm and locking ring 35 is positioned distal to the aneurysm.

An actuator may be operatively coupled to outer sheath 142, graft 114, inner sheath 134 and/or support stent 126. The actuator is activated, as described in greater detail below, to deploy graft 114 from the delivery configuration to a deployed configuration at the lesion site, as shown in FIG. 15. The actuator may include a handle configured to retract outer sheath 142 and deploy graft 114. In this example, the actuator is also operatively coupled to inner sheath 134 and configured to retract inner sheath 134 to deploy support stent 126.

With the deployed graft 114 properly positioned at the lesion site, inner sheath 134 is retracted from about support stent 126 for facilitating expansion of support stent 126 from the compressed delivery configuration to the expanded deployed configuration, as shown in FIG. 18. In the deployed configuration, an outer surface of support stent 126 contacts an inner surface of the graft 114. As shown in FIGS. 16 and 17, support member 136 may be positioned about wire lumen 132 and contacts support stent 126 as inner sheath 134 is retracted to prevent or limit undesirable movement of support stent 126 with respect to the lesion site and maintain support stent 126 positioned at the lesion site. Support member 136 is movable in the proximal direction along the guide wire to contact support stent 126 as inner sheath 134 is retracted in the opposing distal direction as shown by directional arrow 144 (FIG. 14). With graft 114 and support stent 126 deployed at the lesion site, the guide wire is retracted from within the vessel.

“Bottom-Up” Deployment

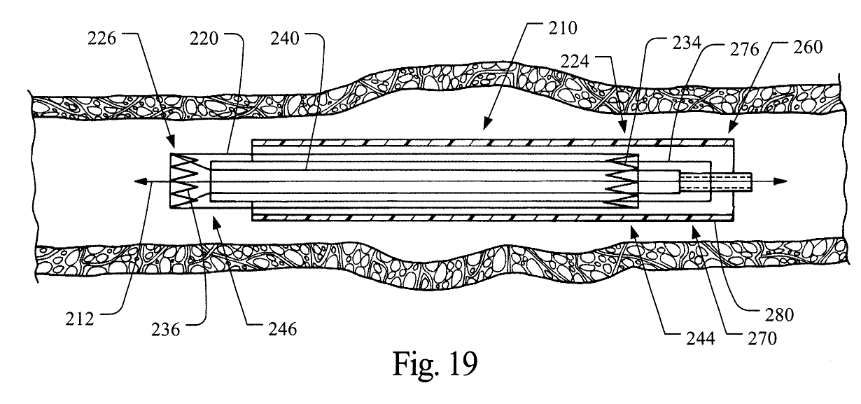

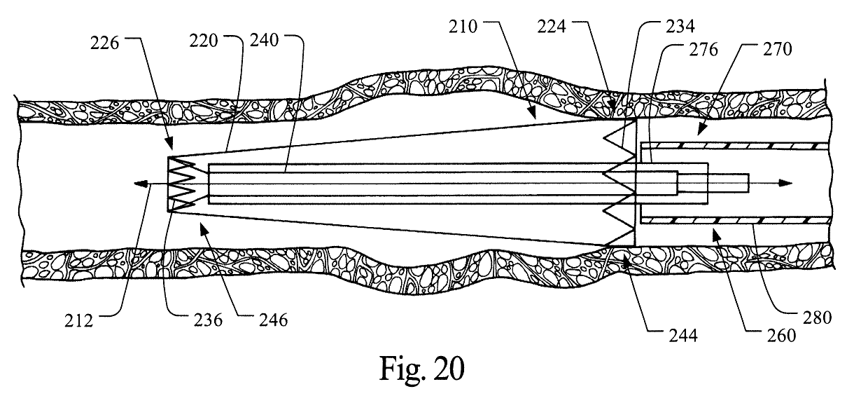

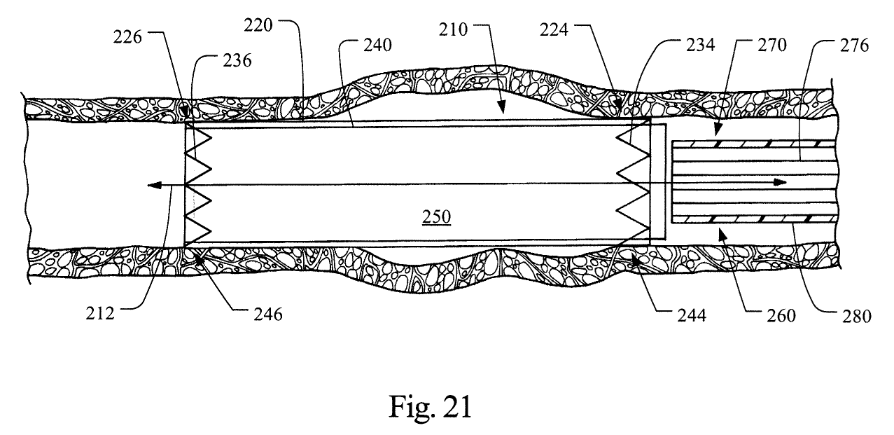

Referring to FIGS. 19-21, an apparatus 260 for delivering stent graft 210 to a lesion site during an endovascular procedure is provided. In one example, outer sheath 280 covers at least a portion of graft 220 during delivery of stent graft 210 to the lesion site. Further, inner sheath 276 is positioned within outer sheath 280 and covers at least a portion of support stent 240 during delivery of stent graft 210 to the lesion site. At the lesion site, outer sheath 280 is movable in a distal direction with respect to longitudinal axis 212 to at least partially expose and/or deploy graft 220. With graft 220 at least partially deployed, distal ring 234 contacts and/or anchors to the interior wall surface of the vessel. Inner sheath 276 is independently movable in the distal direction with respect to longitudinal axis 212 to deploy graft 220 and at least partially expose and/or deploy support stent 240. With support stent 240 at least partially deployed, anchor stent 236 is anchored to the interior wall surface. Support stent 240, including freely movable distal end 244, expands in an outward radial direction with respect to longitudinal axis 212 to contact an inner surface of graft 220 and form or define passage 250.

A method for deploying a stent or stent graft with respect to a lesion site during an endovascular procedure is provided. During the endovascular procedure, a small incision into the patient's skin is made above the femoral artery. The surgeon guides a guide wire into the femoral artery and advances the guide wire through the tortuous vascular structure to the aneurysm, e.g., the lesion site. In this example, stent graft 210 is loaded into delivery device 270. Delivery device 270 is inserted over the guide wire and inserted into the femoral artery to advance stent graft 210 to the lesion site. Delivery device 270 is configured to retain stent graft 210 in a compressed or delivery configuration during delivery of stent graft 210 to the lesion site. Imaging equipment, such as an angiogram imaging system, may be used to facilitate proper positioning of stent graft 210 with respect to the lesion site. Delivery device 270 carries stent graft 210 in the delivery configuration for facilitating advancement of stent graft 210 through the vascular structure, including the blood vessels.

With stent graft 210 positioned at or near the lesion site, the surgeon is able to move delivery device 270 in a proximal direction and/or a distal direction with respect to a position of the patient's heart to position distal ring 234 of stent graft 210 at a desired distal anchoring location with respect to the lesion site. Outer sheath 280 may be partially withdrawn to partially deploy proximal end 226 of graft 220 before moving delivery device 270 to position locking ring 234. Outer sheath 280 is moved in the distal direction to withdraw outer sheath 280 from delivery device 270 and deploy distal end 224 of graft 220 including locking ring 234. Locking ring 234 moves radially outwardly with respect to longitudinal axis 212 to contact the interior wall surface of the vessel at the distal anchor location. Locking ring 234 contacts and/or is anchored to the interior wall surface. Locking ring 234 may contact and/or be anchored to the interior wall surface proximal to an artery, such as the celiac artery, to prevent or limit obstruction of blood flow through the artery.

With locking ring 234 anchored at the distal anchoring location, inner sheath 276 is moved in the distal direction to withdraw inner sheath 276 from delivery device 270 and deploy proximal end 226 of graft 220 including anchor stent 236 and proximal end 246 of support stent 240. Anchor stent 236 moves radially outwardly with respect to longitudinal axis 212 to contact the interior wall surface of the vessel at a proximal anchor location. Anchor stent 236 is then sealingly anchored to the interior wall surface. For example, anchor stent 236 is positioned and anchored distal to the right carotid artery to prevent or limit obstruction of blood flow through the carotid artery. Locking ring 234 and anchor stent 236 may be anchored to the interior wall surface of the vessel to form a seal between the outer surface of locking ring 234, anchor stent 236 and the interior wall surface such that blood flows through passage 250 formed in stent graft 210 in the deployed configuration without allowing blood flow between the outer surface of graft 220 and the interior wall surface. Upon deployment of stent graft 210 with respect to the lesion site, delivery device 270 is withdrawn from the lesion site through the femoral artery.

Alternatively, outer sheath 280 is partially deployed to position retaining locking ring 234. Outer sheath 280 and inner sheath 276 are withdrawn substantially simultaneously to deploy locking ring 234 and anchor stent 236.

Capture Mechanism

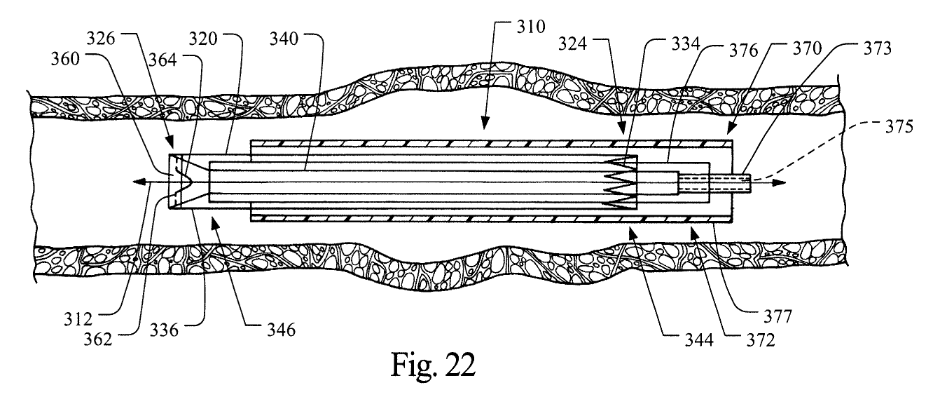

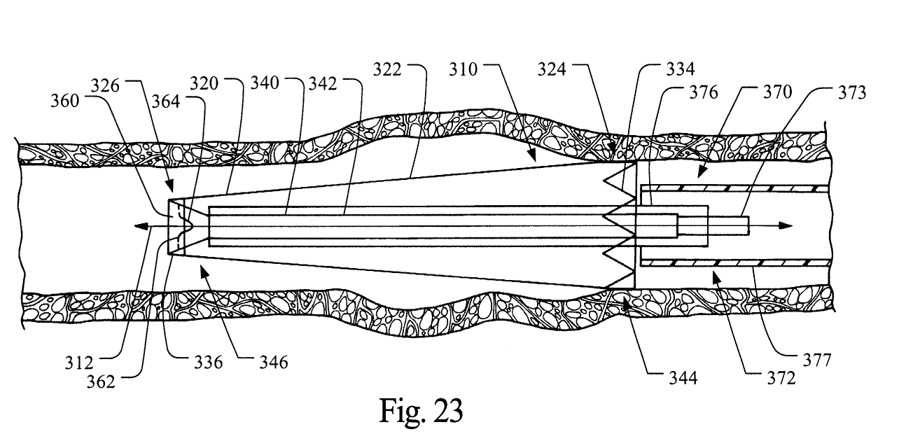

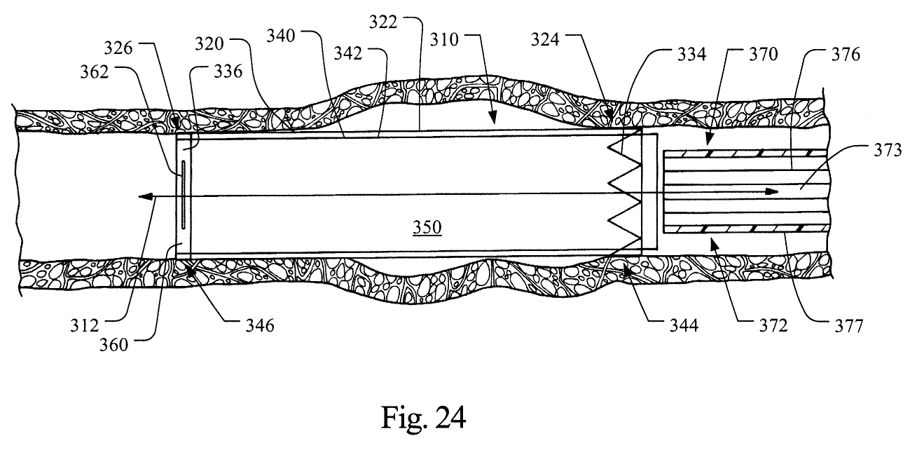

As shown in FIGS. 22-24, stent graft 310 may include a capture mechanism 360 operatively coupled to graft 320 and/or support stent 340. Capture mechanism 360 may be coupled or attached to graft proximal end 326 and/or support stent proximal end 346. Capture mechanism 360 is initially configured to retain graft proximal end 326 in the delivery configuration. As described in greater detail below, capture mechanism 360 is actuatable to release graft proximal end 326 for facilitating radial expansion of graft 320 and/or support stent 340 as the proximal end of stent graft 310 is deployed to the deployed configuration.

Capture mechanism 360 may include an integrated string 362 (as shown in FIGS. 22-24) forming a plurality of string loops 364 coupled to proximal end 326. String 362 may include a plurality of string loops 364 sewn into or otherwise coupled to anchor stent 336. String 362 is movable with respect to proximal end 326 for facilitating retaining proximal end 326 in the delivery configuration and allowing proximal end 326 to move toward the deployed configuration. In this example, a length of each string loop 364 may be made shorter or longer to decrease or increase, respectively, a cross-sectional area of the proximal end of stent graft 310. Further, each string loop 364 is initially operatively coupled to an inner sheath of a delivery device, as described in greater detail below. More specifically, each string loop 364 is coupled to a corresponding capture wire coupled to the inner sheath.

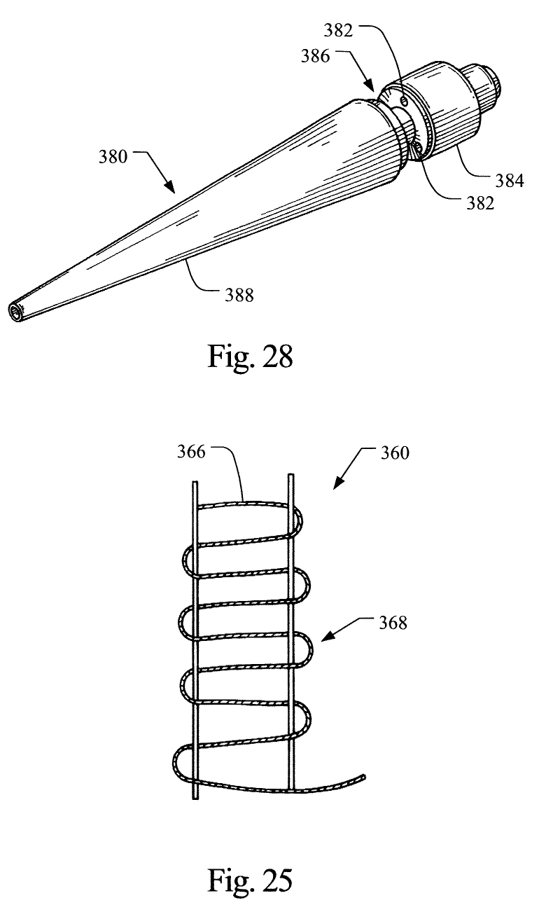

Alternatively, capture mechanism 60 may include a string 366 (as shown in FIG. 25), wrapped about an outer surface of stent graft 310. String 366 may include, for example, suture ribbons, filaments, yarns, threads, wires, strands, as well as any suitable alternative. String 366 may include a plurality of locking knots 368 configured to initially retain graft proximal end 326 in the delivery configuration. In one example, string 366 is initially operatively coupled to the inner sheath, such as by being releasably coupled to the capture wires, and configured to release graft proximal end 326 for facilitating radial expansion of graft proximal end 326 toward the deployed configuration. In this example, string 366 is initially operatively coupled to the inner sheath of a delivery device and releasable from the inner sheath to release the graft proximal end.

Referring further to FIGS. 25-28, an apparatus 370 for delivering stent graft 310 to and deploying stent graft 310 at a lesion site during an endovascular procedure is provided. Apparatus 370 may include stent graft 310 (as shown in FIGS. 22-24) and a delivery device 372 defining a longitudinal axis 373. Delivery device 372 is configured to deliver stent graft 310 to the lesion site within the blood vessel and deploy stent graft 310 at the lesion site. In one example, delivery device 372 may include a wire lumen 374, extending generally along longitudinal axis 373 and defining a passage 375 (shown in FIG. 22) configured to receive a guide wire (not shown) and advance delivery device 372, as well as stent graft 310, to the lesion site. An inner sheath 376 is positioned about wire lumen 374 to contact at least a portion of an outer surface of wire lumen 374. Inner sheath 376 is movable in a proximal direction and a distal direction with respect to wire lumen 374 and longitudinal axis 373. An outer sheath 377 is positioned about inner sheath 3766 to contact at least a portion of inner sheath 376. Outer sheath 377 is independently movable in the proximal direction and the distal direction with respect to wire lumen 374 and inner sheath 376 along longitudinal axis 373.

In one example, outer sheath 377 covers at least a portion of the length of graft 320 during delivery of stent graft 310 to the lesion site. Further, inner sheath 376 is positioned within outer sheath 377 and covers at least a portion of the length of support stent 340 during delivery of stent graft 310 to the lesion site. At the lesion site, outer sheath 377 is movable in a distal direction with respect to longitudinal axis 373 to at least partially expose and deploy graft 320. In this example, with graft 320 at least partially deployed, distal ring 334 is anchored to the interior wall surface of the vessel. Inner sheath 376 is independently movable in the distal direction with respect to longitudinal axis 373 to at least partially expose and deploy support stent 340. With support stent 340 at least partially deployed, anchor stent 336 may be anchored to the interior wall surface. Support stent 340, including freely movable distal end 344, expands in an outward radial direction with respect to longitudinal axis 373 to contact an inner surface of graft 320 and form or define passage 375.

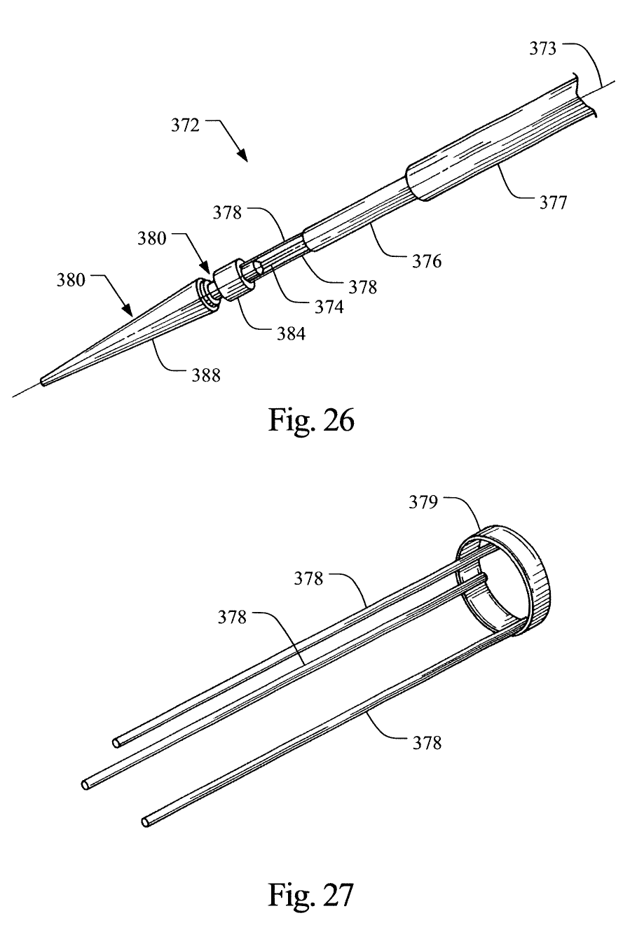

In one example, capture mechanism 360 is initially configured to retain graft proximal end 326 in the delivery configuration. Capture mechanism 360 is actuatable to release graft proximal end 326 for facilitating radial expansion of graft 320. As shown in FIG. 26, a plurality of capture wires 378 are coupled to inner sheath 376. Each capture wire 378 is coupled at a distal end to inner sheath 376 and releasably coupled at an opposing proximal end to capture mechanism 360. In one example, each capture wire 378 is coupled at a distal end to a ring 379, as shown in FIG. 27. Ring 379 is integrated with or coupled to inner sheath 376 using a suitable coupler, such as a string and/or another suitable coupler. Further each capture wire 378 may be releasably coupled at the proximal end to a corresponding string loop 364 formed by integrated string 362 of capture mechanism 360.

Where the capture mechanism 360 may include a string 366 wrapped about an outer surface of stent graft 310, string 346 may be operatively coupled to each capture wire 378. More specifically, string 366 may include a plurality of locking knots 368 initially configured to retain graft proximal end 326 in a delivery configuration, as shown in FIG. 25. String 366 is configured such that locking knots 368 decouple from each capture wire 378 for facilitating releasing graft proximal end 326 to allow proximal end 326 to move radially outward toward the deployed configuration.

Referring further to FIGS. 26 and 28, delivery device 372 may include a nose cone 380 positioned proximal to outer sheath 377 and inner sheath 376. Nose cone 380 may include a plurality of capture wire channels 382 defined within a shaft portion 384 of nose cone 380. In one example, each capture wire channel 382 is positioned radially about and extends parallel to longitudinal axis 373 of deliver device 372. Each capture wire channel 382 may be radially positioned at about 120° with respect to adjacent capture wire channels 382. Any suitable number of capture wire channels 382 may be defined within shaft portion 384 such that a suitable number of capture wires 378 may be fed through a corresponding capture wire channel 382 and releasably coupled to a corresponding string loop 364 formed in capture mechanism 360.

In one example, integrated string 362 forms three (3) string loops 364. Alternatively, integrated string 362 may form at least six (6) string loops 364 to twenty-four (24) string loops 364. Any suitable number of string loops 364 (and corresponding capture wires 378) may be provided to retain the proximal end of stent graft 310 in the delivery configuration or a partially deployed configuration, as desired, without undesirably increasing the loading profile. A plurality of string loops 364 facilitates uniform capturing of graft proximal end 326 and/or uniform releasing of graft proximal end 326 at the desired proximal anchor location for facilitating proper placement of stent graft 310 with respect to the lesion site.

As shown in FIGS. 26 and 28, a string capture groove 386 is defined within nose cone 380 between shaft portion 384 and a lead portion 388 of nose cone 380. String capture groove 386 extends radially about nose cone 380 and substantially perpendicular to longitudinal axis 373. String capture groove 386 intersects each capture wire channel 382 to provide communication between each capture wire channel 382 and string capture groove 386. In one example, each capture wire 378 extends through corresponding capture wire channel 382, from a distal end to a proximal end of capture wire channel 382, and into string capture groove 386. Each string loop 364 formed by capture mechanism 360 is releasably coupled within string capture groove 386 to a corresponding capture wire 378.

In this example, outer sheath 377 is movable in a distal direction along longitudinal axis 373 to deploy graft distal end 324. With graft distal end 324 deployed, distal ring 334 is anchored to the interior wall surface of the vessel. Inner sheath 376 is then movable in a distal direction along longitudinal axis 373 to deploy graft proximal end 326 and/or anchor stent 336. As inner sheath 376 is moved in the distal direction, each capture wire 378 is decoupled from corresponding string loop 364. As each capture wire 378 is decoupled from string loop 364, proximal end 326 of graft 320 moves radially outward toward the deployment configuration. By retaining proximal end 326 in the delivery configuration or a partially deployed configuration as graft distal end 324 is deployed, proximal end 326 can be accurately positioned before stent graft 310 is completely deployed and anchored to the interior wall surface of the vessel.

Referring further to FIG. 25, locking knots 368 of capture ribbon 366 are initially releasably coupled to each capture wire 378. As inner sheath 376 is moved in the distal direction, each capture wire 378 is decoupled from string 366. As each capture wire 378 is decoupled from string 366, proximal end 326 of graft 320 moves radially outward toward the deployment configuration. By retaining proximal end 326 in the delivery configuration or a partially deployed configuration as graft distal end 324 is deployed, proximal end 326 can be accurately positioned before stent graft 310 is completely deployed and anchored to the interior wall surface of the vessel.

In one example, the method may include initially retaining the proximal end of stent graft 310 in the delivery configuration as outer sheath 377 is withdrawn to deploy the distal end of stent graft 310 including distal ring 334. Distal ring 334 is anchored to the vessel wall. Inner sheath 376 of delivery device 372 is then withdrawn to deploy the proximal end of stent graft 310 including anchor stent 336, and anchor stent 336 is anchored to the vessel wall at the proximal anchor location.

In this example, capture mechanism 360 is operatively coupled to the proximal end of stent graft 310 and to a plurality of capture wires 378, which are independently coupled to inner sheath 376. Capture mechanism 360 initially retains graft proximal end 326 in the delivery configuration. With the proximal end of stent graft 310 retained in the delivery configuration, the proximal end of stent graft 310 is positioned with respect to the lesion site at a desirable proximal anchor location. Capture mechanism 360 is actuated to release graft proximal end 326 for facilitating radially expanding the proximal end of the stent graft. Inner sheath 376 is withdrawn to deploy the proximal end of stent graft 310 such that capture wires 378 coupled to the proximal end of inner sheath 376 are released from capture mechanism 360.

Capture mechanism 360 may include integrated string 362 coupled to graft proximal end 326. Integrated string 362 is sewn into graft proximal end 326 and/or anchor stent 336 to form string loops 364. Each capture wire 378 is releasably coupled to a corresponding string loop 364. Inner sheath 376 is moved in a distal direction to decouple each capture wire 378 from a corresponding string loop 364 formed on capture mechanism 360 to actuate capture mechanism 360 and release graft proximal end 326.

In one example, nose cone 380 of delivery device 372 defines a suitable number of capture wire channels 382. Each capture wire channel 382 is positioned radially about and extends parallel to longitudinal axis 373 of deliver device 372. String capture groove 386 is defined within nose cone 380. String capture groove 386 extends radially about nose cone 380 and substantially perpendicular to longitudinal axis 373. String capture groove 386 intersects each capture wire channel 382 to provide communication between each capture wire channel 382 and string capture groove 386. Each capture wire 378 is initially fed through a corresponding capture wire channel 382 and into string capture groove 386, wherein each capture wire 378 is coupled within string capture groove 386, to a corresponding string loop 364 formed in capture mechanism 360.

Delivery Device Actuator

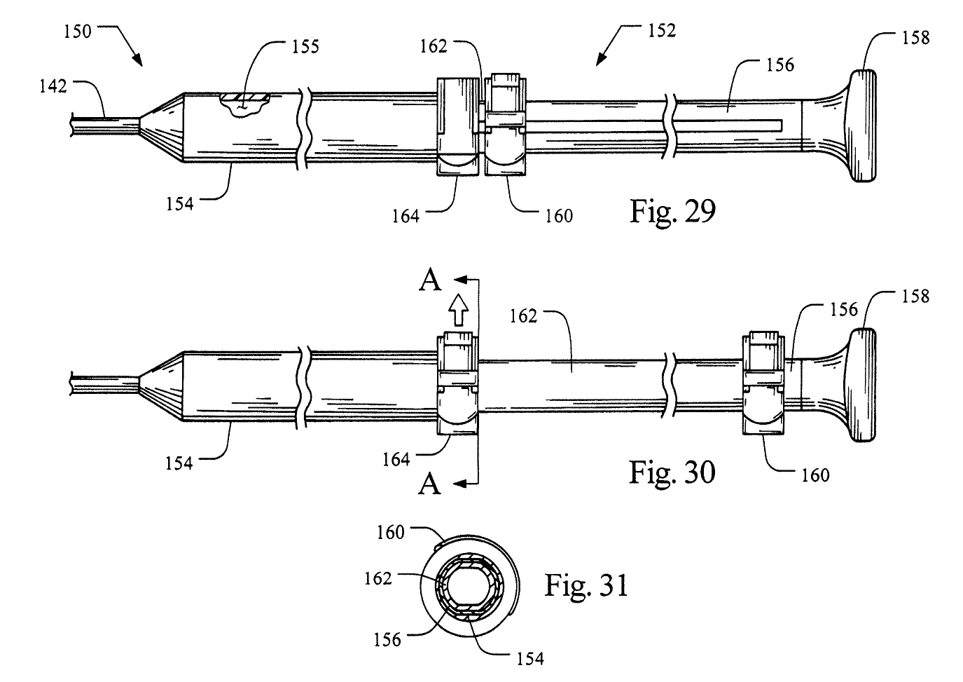

Referring to FIGS. 29-31, delivery system 130 may include an actuator 150. Actuator 150 has a handle 152 operatively coupled to inner sheath 134 and outer sheath 142. Handle 152 may include a housing 154 defining a chamber 155. Handle 152 further may include an outer sheath retraction tube 156 that is slidably positioned within chamber 155 and coupled at a proximal end to outer sheath 142. A retraction element 158 is coupled to a distal end of outer sheath retraction tube 156 for facilitating moving outer sheath retraction tube 156 with respect to housing 154. As shown in FIG. 29, outer sheath retraction tube 156 is slidably movable with respect to housing 154 in the distal direction to retract outer sheath 142 and deploy graft 114. In this example, as outer sheath 142 is retracted, graft 114 expands in a radial direction to contact an interior surface of the vessel wall. Alternatively, actuator 150 is activated to deploy graft 114 from the delivery configuration to a deployed configuration at the lesion site.

As shown in FIG. 29, a first locking element 160 is positioned about outer sheath retraction tube 156 and configured to lock outer sheath retraction tube 156 in a locked position to prevent or limit movement of outer sheath retraction tube 156 within housing 154 as stent graft 110 is delivered and/or positioned at the lesion site. With stent graft 110 properly positioned at the lesion site, first locking element 160 is unlocked and outer sheath retraction tube 156 is drawn in a distal direction with respect to housing 154 to retract outer sheath 142.

Handle 152 also may include an inner sheath retraction tube 162 that is slidably positioned about outer sheath retraction tube 156, as shown in FIG. 30. Inner sheath retraction tube 162 is coupled at a proximal end to inner sheath 134 and first locking element 160 is coupled to an opposing distal end of inner sheath retraction tube 162. As shown in FIG. 30, inner sheath retraction tube 162 is slidably movable with respect to outer sheath retraction tube 156 in a distal direction to retract inner sheath 134 and deploy support stent 126. In one example, a second locking element 164 is coupled to housing 154 and configured to lock inner sheath retraction tube 162 in a locked position to prevent or limit movement of inner sheath retraction tube 162 with respect to outer sheath retraction tube 156 as stent graft 110 is delivered and/or positioned at the lesion site. With stent graft 110 properly positioned at the lesion site, second locking element 164 is unlocked and inner sheath retraction tube 162 is drawn in a distal direction with respect to outer sheath retraction tube 156 to retract inner sheath 134.

Referring to FIG. 31, outer sheath retraction tube 156 and/or inner sheath retraction tube 162 has a non-circular cross-sectional area configured to prevent or limit undesirable rotational movement of outer sheath retraction tube 156 and/or inner sheath retraction tube 162.

In this example, with stent graft 110 properly positioned at the lesion site, first locking element 160 is unlocked. Retraction element 158, and outer sheath retraction tube 156 coupled thereto, is slid in a distal direction to retract outer sheath 142 to deploy graft 114 at a lesion site. Second locking element 164 is then unlocked and first locking element 160, and inner sheath retraction tube 162 coupled thereto, is slid in the distal direction to retract inner sheath 134 positioned about support stent 126. As inner sheath 134 is retracted, support stent 126 expands from the compressed delivery configuration to an expanded deployed configuration. In the deployed configuration, an outer surface of support stent 126 contacts an inner surface of graft 114. First locking element 160 and second locking element 164 may be unlocked and retraction element 158 and first locking element 160 are slid in the distal direction substantially simultaneously to deploy graft 114 and support stent 126 at the lesion site.

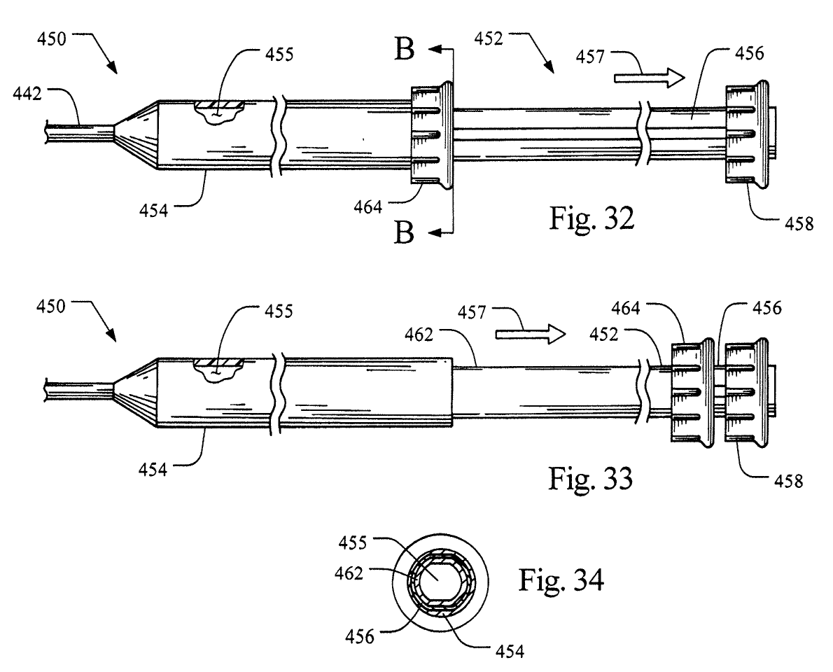

Referring to FIGS. 32-41, an actuator 450 may include a handle 452 operatively coupled to inner sheath 434 and outer sheath 442. Handle 452 may include a housing 454 defining a chamber 455. Handle 452 also may include an outer sheath retraction tube 456 that is slidably positioned within housing 454 and coupled to a distal end of outer sheath 442. A first retraction element 458 is coupled to a distal end of outer sheath retraction tube 456 for facilitating moving outer sheath retraction tube 456 with respect to housing 454. As shown in FIG. 32, outer sheath retraction tube 456 is slidably movable with respect to housing 454 in a distal direction as shown by directional arrow 457 to retract outer sheath 442 and deploy graft 414. In one example, first retraction element 458 is configured to lock outer sheath retraction tube 456 in a locked position to prevent or limit movement of outer sheath retraction tube 456 within housing 454.

As shown in FIG. 32, outer sheath retraction tube 456 is slidably movable with respect to housing 454 in the distal direction to retract outer sheath 442 and deploy graft 414. In this example, as outer sheath 442 is retracted, graft 414 expands in a radial direction to contact an interior surface of the vessel wall. Alternatively, actuator 450 is activated to deploy graft 414 from the delivery configuration to a deployed configuration at the lesion site.

First retraction element 458 is positioned about outer sheath retraction tube 556 and configured to lock outer sheath retraction tube 456 in a locked position to prevent or limit movement of outer sheath retraction tube 456 within housing 454 as stent graft 410 is delivered and/or positioned at the lesion site. With stent graft 410 properly positioned at the lesion site, first retraction element 458 is rotated with respect to outer sheath retraction tube 456 to unlock outer sheath retraction tube 456. Outer sheath retraction tube 456 is then drawn in the distal direction with respect to housing 454 to retract outer sheath 442.

Handle 452 also may include an inner sheath retraction tube 462 that is slidably positioned about outer sheath retraction tube 456, as shown in FIG. 33. Inner sheath retraction tube 462 is coupled at a proximal end to inner sheath 434 and a second retraction element 464 is coupled to an opposing distal end of inner sheath retraction tube 462. As shown in FIG. 33, inner sheath retraction tube 462 is slidably movable with respect to outer sheath retraction tube 456 in a distal direction to retract inner sheath 434 and deploy support stent 426. In one example, second retraction element 464 is configured to lock inner sheath retraction tube 462 in a locked position to prevent or limit movement of inner sheath retraction tube 462 with respect to outer sheath retraction tube 456 as stent graft 410 is delivered and/or positioned at the lesion site. With stent graft 410 properly positioned at the lesion site, second retraction element 464 is rotated to an unlocked position and inner sheath retraction tube 462 is drawn in the distal direction with respect to outer sheath retraction tube 456 to retract outer sheath 442.

Referring to FIG. 34, outer sheath retraction tube 456 and/or inner sheath retraction tube 462 may have a non-circular cross-sectional area configured to prevent or limit undesirable rotational movement of outer sheath retraction tube 456 and/or inner sheath retraction tube 460.

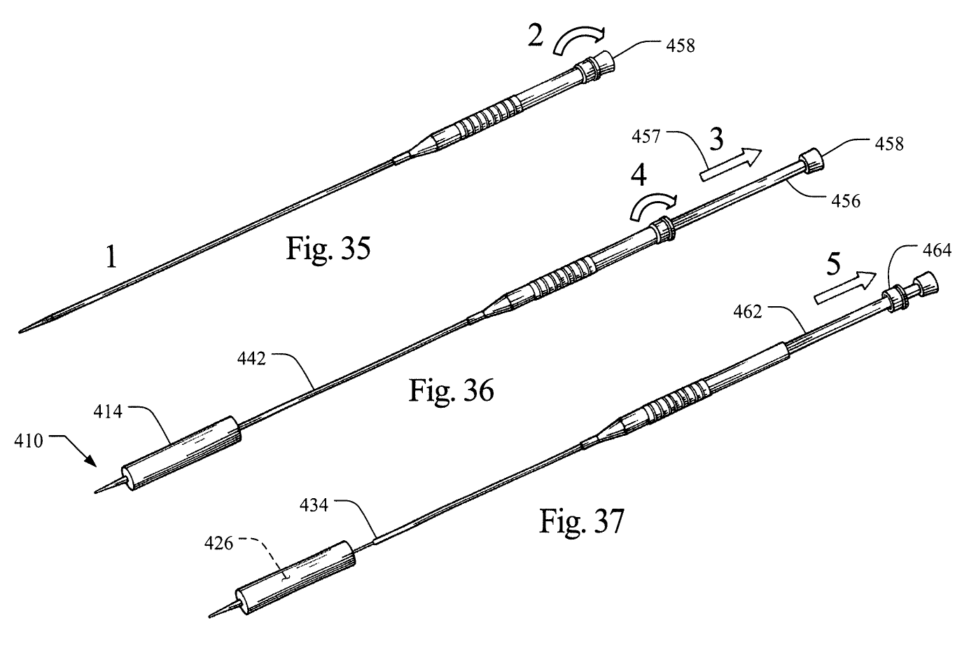

Referring further to FIGS. 35-41, with stent graft 410 properly positioned at the lesion site, first retraction element 458 is rotated to an unlocked position, as shown in FIG. 35. Outer sheath retraction tube 456 is slid with respect to housing 454 in distal direction 457 to retract outer sheath 442 positioned about anchor stent 414 in the delivery configuration to deploy graft 414 at a lesion site, as shown in FIG. 36. Second retraction element 464 is then rotated to an unlocked position and inner sheath retraction tube 462 is slid in the distal direction to retract inner sheath 434 positioned about support stent 426 in a compressed delivery configuration, as shown in FIG. 37. As inner sheath 434 is retracted, support stent 426 expands from the compressed delivery configuration to an expanded deployed configuration. In the deployed configuration, an outer surface of support stent 426 contacts an inner surface of graft 414. First retraction element 458 and second retraction element 464 may be unlocked and outer sheath retraction tube 456 and inner sheath retraction tube 462 slid in the distal direction substantially simultaneously to deploy graft 414 and support stent 426 at the lesion site.

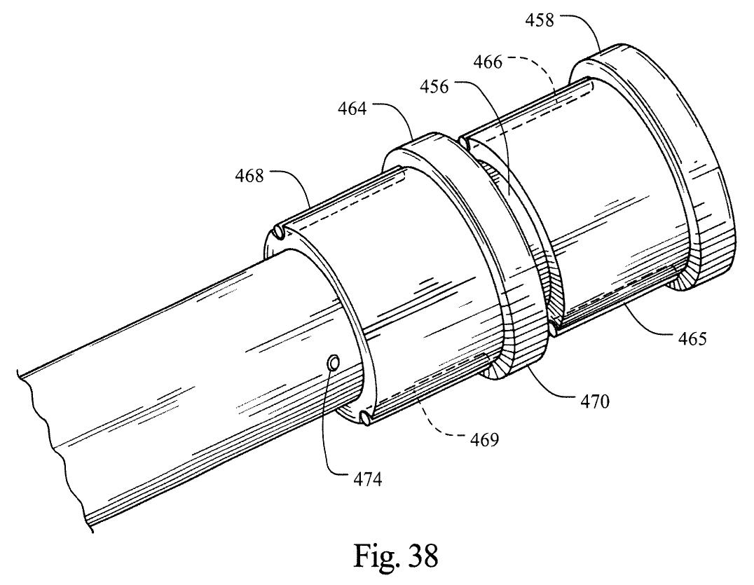

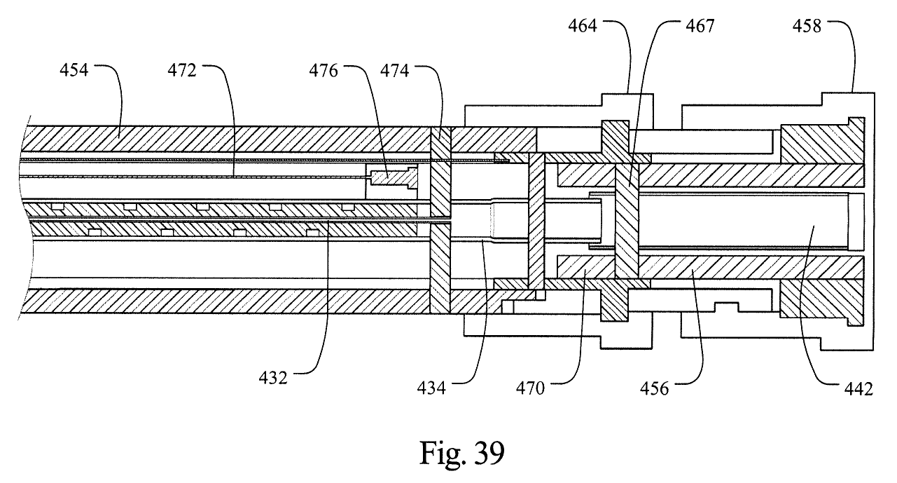

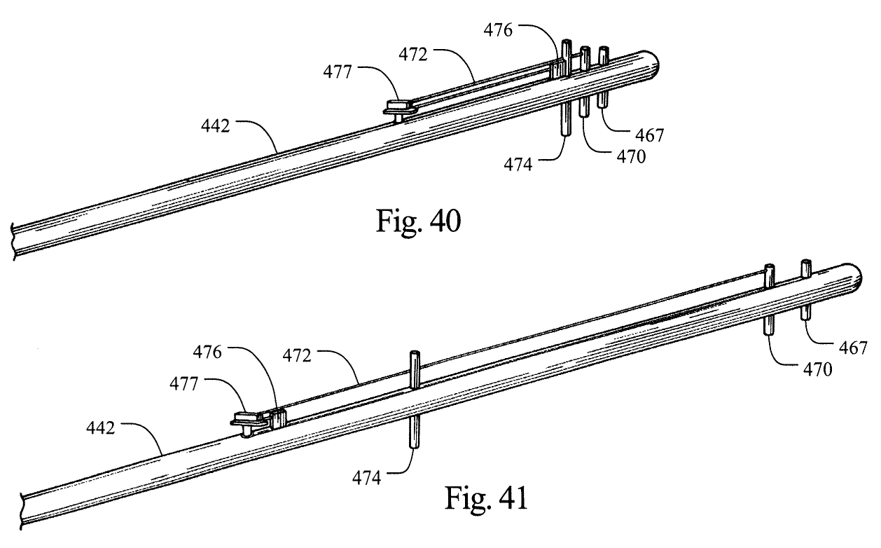

As shown in FIG. 38, first retraction element 458 forms a projection, such as pin 465, that is movably positioned within a slot 466 defined within outer sheath retraction tube 456. First retraction element 458 is rotated such that pin 465 travels along slot 466 to move first retraction element 458 between a locked position and an unlocked position, as shown in FIG. 38. With first retraction element 456 in the unlocked position, outer sheath retraction tube 456 is drawn in the distal direction to retract outer sheath 442. In this example, pin 465 interferes with a post 467 coupled to outer sheath 442, as shown in FIG. 39, to retract outer sheath 442 as outer sheath retraction tube 456 is drawn or pulled in the distal direction. Similarly, second retraction element 464 forms a projection, such as pin 468 shown in FIG. 38, which is movably positioned within a slot 469 defined within inner sheath retraction tube 462. Second retraction element 464 is rotated such that pin 468 travels along slot 469 to move second retraction element 464 between a locked position, as shown in FIG. 38, and an unlocked position. With second retraction element 464 in the unlocked position, inner sheath retraction tube 462 is drawn or pulled in the distal direction to retract inner sheath 434. Referring to FIGS. 38-41, pin 468 interferes with a post 470 coupled to inner sheath 434, as shown in FIG. 39, to retract inner sheath 434 as inner sheath retraction tube 462 is drawn. Further, as shown in FIGS. 39-41, a string 472 couples post 470 through an anchor pin 474 to support member 436. In one example, support member 436 may include a projection, such as a block 476, to which string 472 is coupled. Referring further to FIGS. 40 and 41, string 472 is wrapped about a spindle 477 operatively coupled to housing 454. As inner sheath 434 is retracted in the distal direction, support member 436 coupled to inner sheath 434 by string 472 is moved in an opposing proximal direction to retain support stent 426 properly positioned at the lesion site.

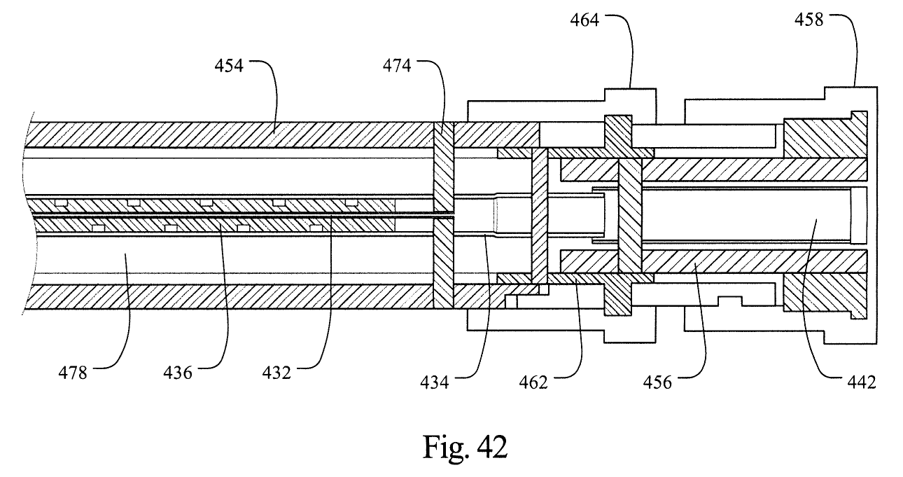

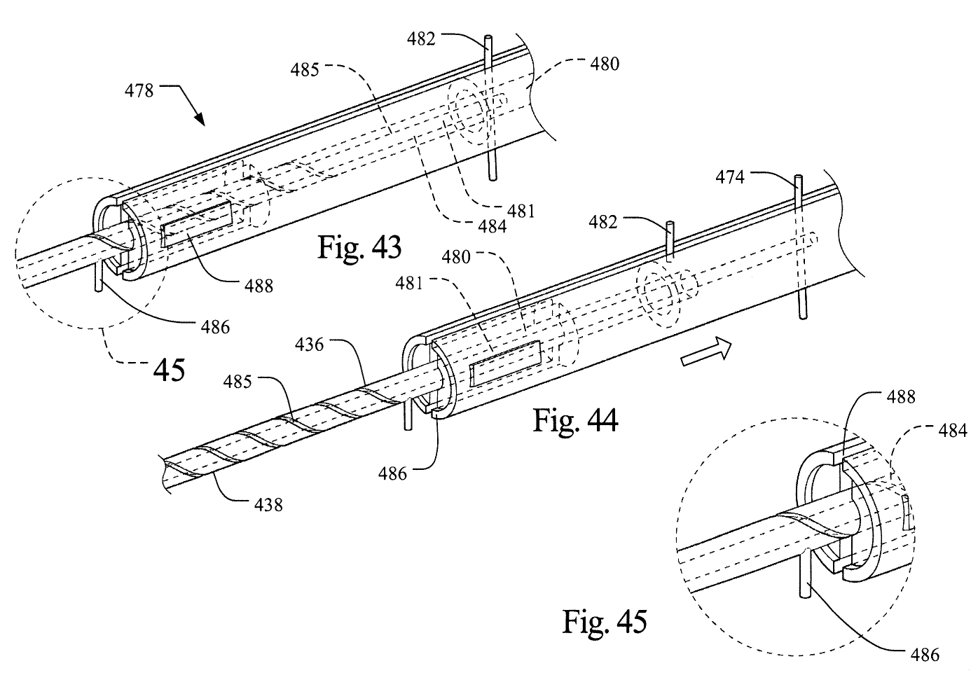

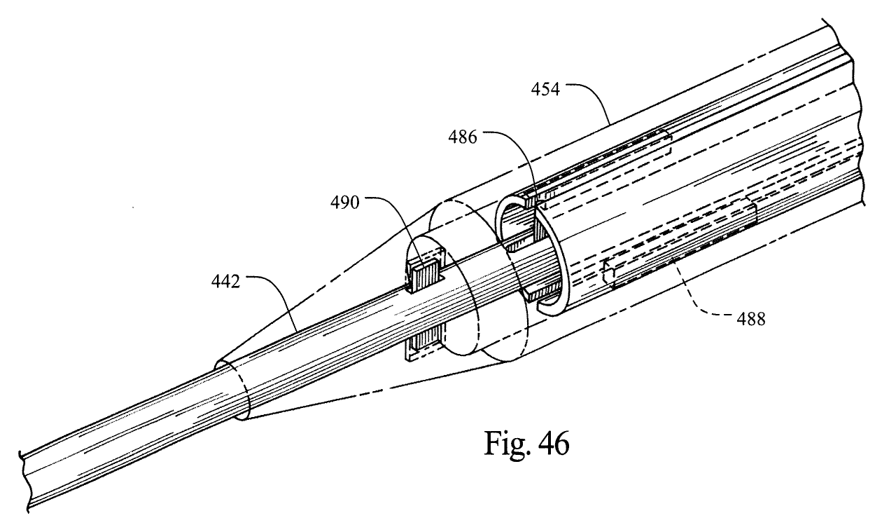

Referring to FIGS. 42-46, inner sheath retraction tube 462 is retracted to activate a cam system 478 that advances support member 436 as inner sheath retraction tube 462 is retracted. In this example, support member 436 may include a first or distal cam portion 480 forming a helical track 481 that cooperates with an advancement pin 482 that is fixedly coupled to housing 454 as support member 436 is advanced in the proximal direction. The cooperation of first cam portion 480 with advancement pin 482 facilitates advancement of support member 436 in the proximal direction. Support member 436 also may include a second or proximal cam portion 484 forming a helical track 485 that cooperates with a rotation pin 486 that is fixedly coupled to housing 454 as support member 436 advances in the proximal direction. The cooperation of second cam portion 484 with rotation pin 486 facilitates rotation of support member 436. In one example, at least one rail 488 is coupled to or formed in housing 454 for facilitating resisting torque stresses and/or rational forces produced by inner sheath retraction tube 462 as inner sheath retraction tube 462 is retracted to activate cam system 478. As shown in FIG. 44, proximal end 438 of support member 436 is coupled to second cam portion 484 such that proximal end 438 does not rotate as support member 436 is advanced. Further, a blade 490 may be mounted with respect to a proximal end of housing 454, as shown in FIG. 46, to cut and/or split outer sheath 442 for facilitating clearing cam system 478 (not shown in FIG. 46) without interfering with cam system 478 as outer sheath 442 is retracted.

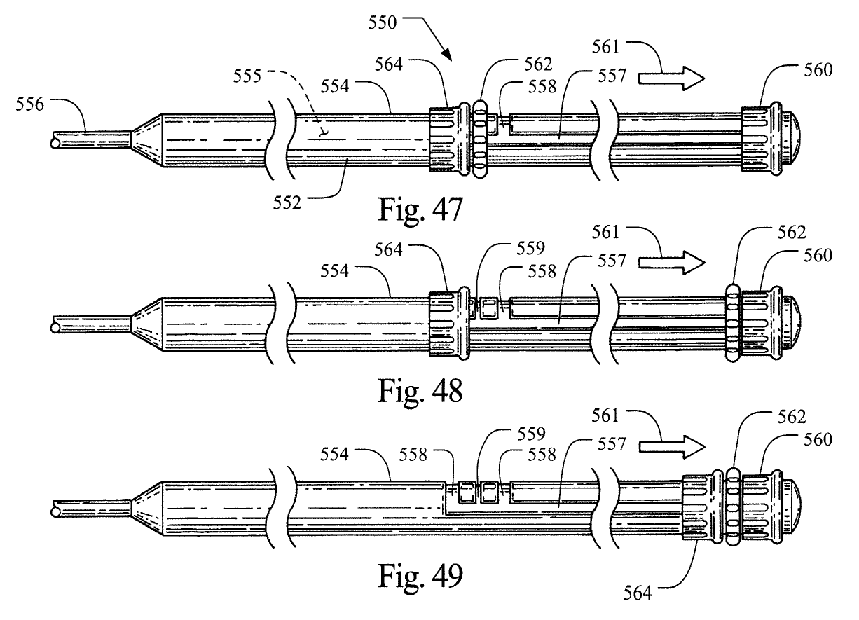

Referring to FIGS. 47-49, an actuator 550 may include a handle 552 operatively coupled to inner sheath 534 and/or outer sheath 542. Handle 552 may include a housing 554 defining a chamber 555. Housing 554 defines an axis 556 and a track 557 along at least a portion of axis 556, as shown in FIG. 47. In one example, track 557 defines or may include at least one locking groove 558 and/or at least one intermediate groove 559.

A first retraction element 560 is positioned about housing 554 and operatively coupled to outer sheath 542. First retraction element 560 is movable, such as by rotating first retraction element 560, between a locked position and an unlocked position. In the locked position, first retraction element 560 is positioned within a first locking groove 558 to prevent or limit movement of outer sheath 542 as stent graft 510 is delivered and/or positioned at the lesion site. With stent graft 510 properly positioned at the lesion site, first retraction element 560 is unlocked and slidably movable within track 557 in a distal direction, as shown by directional arrow 561, to retract outer sheath 542, as shown in FIG. 47.

A second retraction element 562 is positioned about housing 554 and operatively coupled to graft 514. Second retraction element 562 is movable, such as by rotating second retraction element 562, between a locked position and an unlocked position. In the locked position, second retraction element 562 is positioned within intermediate locking groove 559 to prevent or limit movement of graft 514 as stent graft 510 is delivered and/or positioned at the lesion site. As shown in FIG. 48, with stent graft 510 properly positioned at the lesion site, second retraction element 562 is unlocked and slidably movable with respect to housing 554 in the distal direction to deploy graft 514, as described in greater detail below.

A third retraction element 564 is positioned about housing 554 and operatively coupled to inner sheath 534. Third retraction element 564 is movable, such as by rotating third retraction element 564, between a locked position and an unlocked position. In the locked position, third retraction element 564 is positioned within a locking groove 558 to prevent or limit movement of inner sheath 534 as stent graft 510 is delivered and/or positioned at the lesion site. With stent graft 510 properly positioned at the lesion site, third retraction element 564 is unlocked and slidably movable with respect to housing 554 in the distal direction, as shown in FIG. 49, to retract inner sheath 534 and deploy support stent 526.

In this example, with stent graft 510 properly positioned at the lesion site, first retraction element 560 is rotated within corresponding locking groove 558 to unlock first retraction element 560. As shown in FIG. 47, first retraction element 560 is drawn or pulled with respect to housing 554 in the distal direction to retract outer sheath 542 positioned about graft 514 in the delivery configuration. Second retraction element 562 is rotated within intermediate groove 559 and is drawn or pulled with respect to housing 554 in the distal direction, as shown in FIG. 48, to deploy graft 514 at the lesion site. Third retraction element 564 is rotated within corresponding locking groove 558 to unlock third retraction element 564 positioned about housing 554 and operatively coupled to inner sheath 534. Third retraction element 564 is drawn or pulled with respect to housing 554 in the distal direction to retract inner sheath 534 positioned about support stent 526 in a compressed delivery configuration. With inner sheath 534 in the retracted position, support stent 526 is expandable from the compressed delivery configuration to an expanded configuration, wherein an outer surface of support stent 526 contacts an inner surface of graft 514. In one example, first retraction element 560, second retraction element 562 and third retraction element 564 are unlocked and slid in the distal direction substantially simultaneously to deploy graft 514 and support stent 526 at the lesion site.

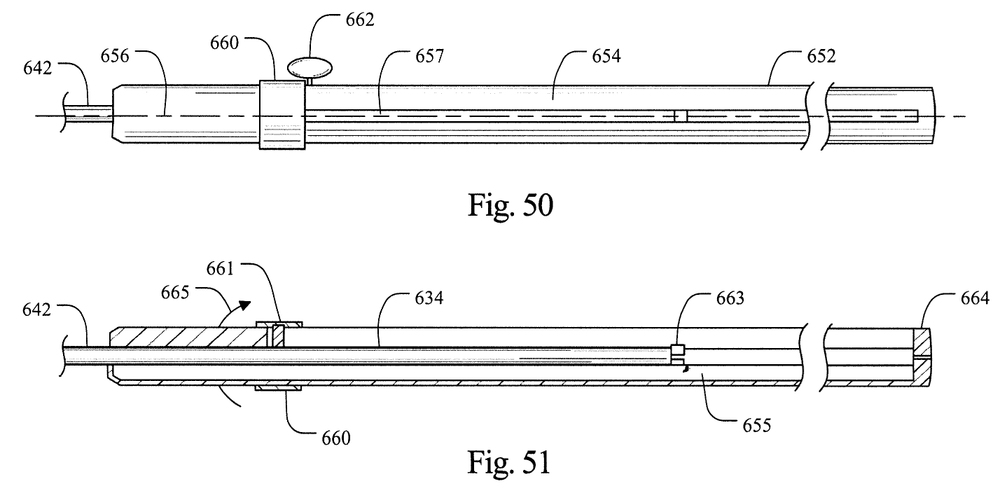

Referring to FIGS. 50-53, an actuator 650 may include a handle 652 operatively coupled to inner sheath 634 and/or outer sheath 642. Handle 652 may include a housing 654 defining a chamber 655 within which inner sheath 634 and/or outer sheath 642 is positionable in the retracted position. Housing 654 further defines an axis 656 and a track 657 along at least a portion of axis 656, as shown in FIG. 50. In one example, track 657 extends through housing 654 and is in communication with chamber 655.

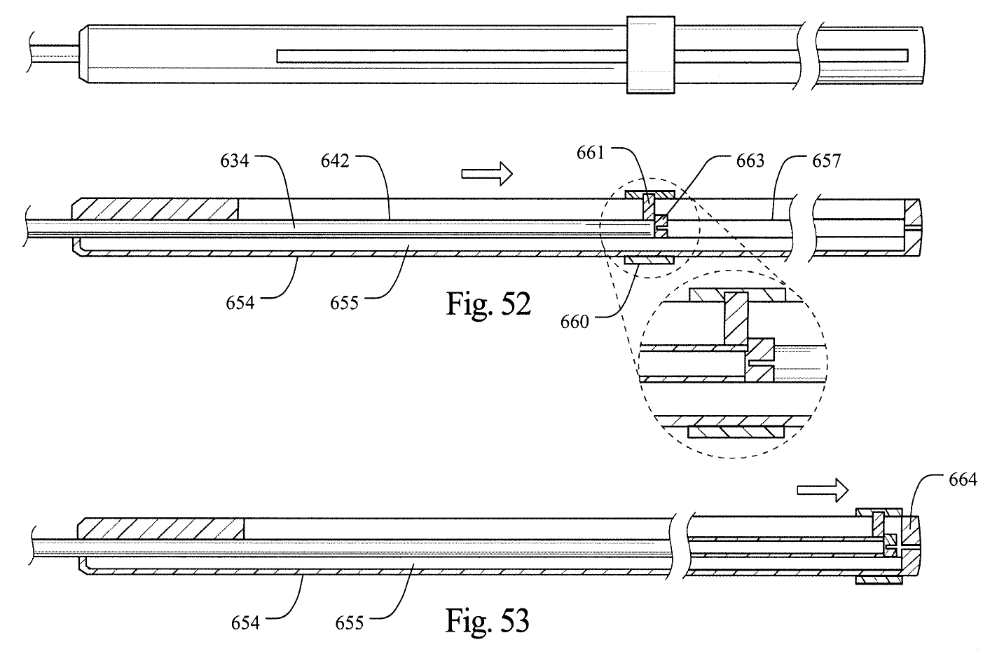

A retraction element 660 is positioned about housing 654 and operatively coupled to outer sheath 642. In one example, a connector 661 couples outer sheath 642 to retraction element 660. As shown in FIG. 51, connector 661 is coupled to outer sheath 642 and extends through track 657 to couple to retraction element 660. Retraction element 660 is retained in an initial position along axis 656 by a locking element 662 that extends into an aperture (not shown) defined by housing 654. Locking element 662 is configured to initially prevent or limit movement of retraction element 660 and/or outer sheath 642 along axis 656. In one example, locking element 662 is removable from within housing 654 to allow retraction element 660 to move along a length of housing 654. Alternatively, locking element 662 is breakable at a coupling point or area with housing 654 to allow retraction element 660 to move along the length of housing 654. Retraction element 660 is rotatable with respect to housing 654 between a locked position and an unlocked position. With locking element 662 removed from the aperture in housing 654 and retraction element 660 rotated to the unlocked position, retraction element 660 is slidably movable with respect to housing 654 in a distal direction between an initial position, as shown in FIG. 50, and a first stop position, as shown in FIG. 52, to retract outer sheath 642. As shown in FIG. 52, at the first stop position connector 661 contacts a connector 663 that is coupled to inner sheath 634. Connector 663 is at least partially positioned within track 657 for facilitating preventing inner sheath 634 from undesirably rotating within chamber 655. In one example, connector 663 is configured to interfere with connector 661 as retraction element 660 is moved from the first stop position to a second or final stop position, as shown in FIG. 53. As retraction element 660 moves toward the final stop position, connector 663 moves along track 657 towards a back stop 664 coupled to and/or integrated with a distal end of housing 654 to retract inner sheath 634.

In one example, with stent graft 610 properly positioned at the lesion site, locking element 662 is removed from housing 654, for example by breaking locking element 662 at the housing coupling area. Retraction element 660 is rotated to an unlocked position. In one example, retraction element 660 is rotated in a rotational direction as shown by directional arrow 665 in FIG. 51. Alternatively, retraction element 660 is configured to rotate in a rotational direction opposite the rotational direction shown in FIG. 51. Retraction element 660 is drawn or pulled with respect to housing 654 in a distal direction between an initial position, as shown in FIG. 51, and the first stop position, as shown in FIG. 52, to retract outer sheath 642 and deploy graft 114 at the lesion site. Retraction element 660 is slidably movable with respect to housing 654 in the distal direction between the first stop position and the final stop position at or near back stop 664, as shown in FIG. 53, to retract inner sheath 634 and deploy support stent 126 at the lesion site. With graft 114 deployed at the lesion site, support stent 126 is deployed such that at least a portion of an exterior surface of support stent 126 contacts at least a portion of an interior surface of graft 114.

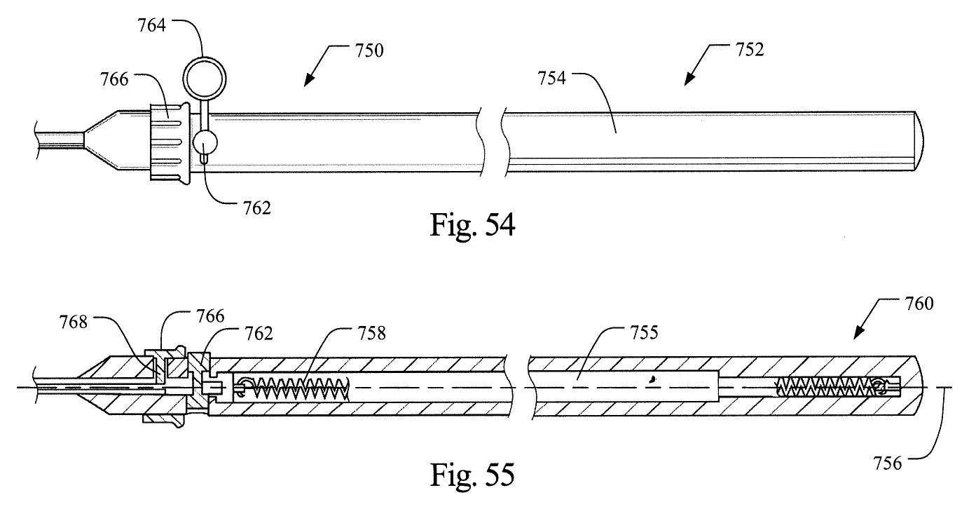

Referring to FIGS. 54-58, an actuator 750 may include a handle 752 operatively coupled to inner sheath 734 and/or outer sheath 742. Handle 752 may include a housing 754 defining a chamber 755 along at least a portion of a length of housing 754 and an axis 756. Referring further to FIGS. 56 and 57, at least a portion of inner sheath 734 and/or outer sheath 742 is slidably movable within chamber 755.

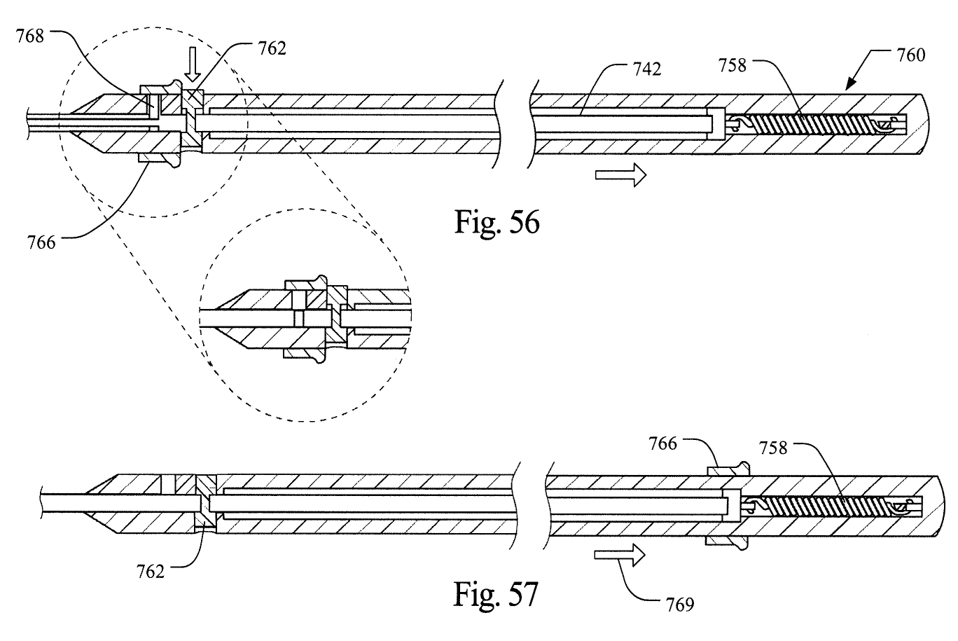

In one example, a biasing element 758, such as a spring, is positioned within chamber 755. Biasing element 758 is coupled at a first end to a distal end 760 of housing 754 and at a second end to outer sheath 742. In this example, biasing element 758 biases outer sheath 742 towards distal end 760. A push button 762 is positioned within and/or coupled to housing 754 and configured to retain outer sheath 742 in a delivery configuration. As shown in FIG. 55, push button 762 extends into chamber 755 to retain outer sheath 742 in the delivery configuration. Push button 762 is movable between a delivery position wherein push button 762 retains outer sheath 742 in the initial delivery configuration and a depressed position for facilitating retracting outer sheath 742. More specifically, push button 762 is configured to lock or interfere with outer sheath 742 to retain biasing element 758 in an extended position, as shown in FIG. 55. In one example, a locking element 764 is configured to retain push button 762 in an initial position. Push button 762 defines a passage through which locking element 764 extends to prevent push button 762 from moving inwardly with respect to housing 754.

With locking element 764 removed, push button 762 is depressed to release outer sheath 742 and allow biasing element 758 to recoil to an inertial position. As biasing element 758 moves toward the inertial position, biasing element 758 biases outer sheath 742 towards distal end 760 to retract outer sheath 742, as shown in FIG. 56, and deploy graft 714. In one example, with outer sheath 742 retracted, inner sheath 734 rotates and partially retracts to allow a portion of support stent 126 to expand. A retraction element 766 is positioned about housing 754 and operatively coupled to inner sheath 734. A connector 768 may couple or engage retraction element 766 to inner sheath 734, as shown in FIGS. 55 and 56. Retraction element 766 may be rotatable with respect to housing 754 between a locked position and an unlocked position. In the unlocked position, retraction element 766 facilitates aligning connector 768 with a slot or track defined within housing 754. In the unlocked position, retraction element 766 is slidably movable with respect to housing 754 in a distal direction to retract inner sheath 734, as shown by directional arrow 769 in FIG. 57.

In one example, a second biasing element 770, such as a spring, is positioned within chamber 755. Biasing element 770 is coupled at a first end to distal end 760 of housing 754 and at a second end to connector 768. In this example, biasing element 770 biases inner sheath 734 towards distal end 760. A second push button (not shown) is positioned within and/or coupled to housing 754 and configured to retain inner sheath 734 in a delivery configuration. The push button may extend into chamber 755 to retain inner sheath 734 in the delivery configuration. The push button is movable between a delivery position, wherein the push button retains inner sheath 734 in the initial delivery configuration, and a depressed position for facilitating retracting inner sheath 734.

In one example, with stent graft 110 properly positioned at the lesion site, locking element 764 is removed from housing 754, which retains push button 762 in an initial position. Push button 762 is pressed to release outer sheath 742 and retract outer sheath 742 to automatically deploy graft 114. By pressing push button 762 to move push button 762 with respect to housing 754, outer sheath 742 is released and spring 758 recoils to retract outer sheath 742. Inner sheath 734 may be partially retracted to partially deploy support stent 726. Retraction element 766 coupled to inner sheath 734 is rotated to unlock retraction element 766 and align connector 768 with a slot formed in outer sheath 742. Retraction element 766 is slid along housing 754 in the distal direction, to retract inner sheath 734 and deploy support stent 126.

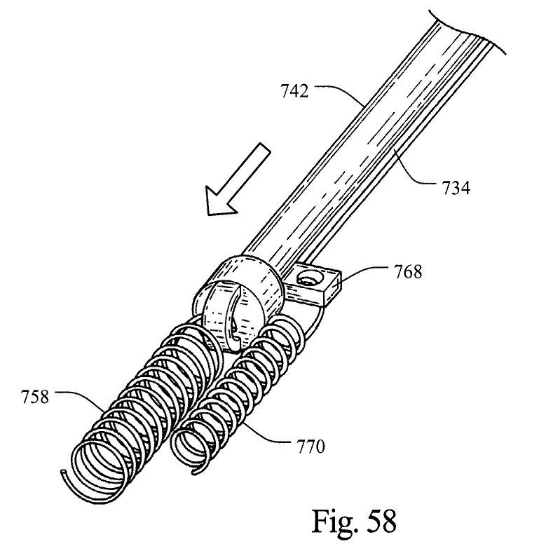

In one example, as shown in FIG. 58, each of outer sheath 742 and inner sheath 734 is coupled to a biasing element 758 and 770, respectively, to bias outer sheath 742 and inner sheath 734 towards distal end 760 of housing 754. Each biasing element 758, 770 is operatively coupled to a corresponding push button that is pressed to release respective biasing elements 758, 770 and retract outer sheath 742 and inner sheath 734. The push buttons may be pressed substantially simultaneously to release and retract outer sheath 742 and inner sheath 734 and deploy stent graph 110.

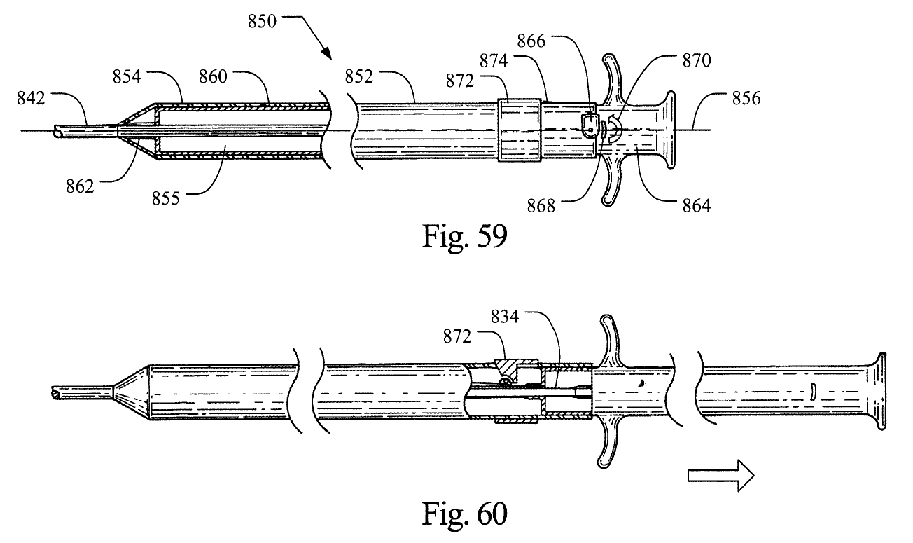

Referring to FIGS. 59-69, an actuator 850 may include a handle 852 operatively coupled to inner sheath 834 and/or outer sheath 842. Handle 852 may include a housing 854 defining a chamber 855 along at least a portion of a length of housing 854 and an axis 856. As shown in FIGS. 59-61, at least a portion of inner sheath 834 and/or outer sheath 842 is slidably movable within chamber 855.

In one example, an outer sheath retraction tube 860 is concentrically positioned within housing 854. Outer sheath retraction tube 860 is movable within housing 854 along axis 856 and configured to retract outer sheath 842. As shown in FIG. 59, outer sheath 842 is coupled about a nipple 862 formed at a proximal end of outer sheath retraction tube 860. Outer sheath retraction tube 860 transitions into or is coupled to an outer sheath retraction element 864. Outer sheath retraction element 864 is movable along axis 856 to move outer sheath retraction tube 860 in a distal direction along axis 856 and retract outer sheath 842. As shown in FIG. 59, an outer sheath locking element 866 is coupled to housing 854 and is configured to prevent or limit movement of outer sheath 842 with respect to axis 856. In one example, outer sheath locking element 866 is rotatably coupled to housing 854. In a locked position, outer sheath locking element 866 contacts a projection 868, such as an arcuate wall, formed on an outer surface of outer sheath retraction grip 864. Outer sheath locking element 866 is rotatable in a rotational direction as shown by directional arrow 870 in FIG. 59 to an unlocked position to release outer sheath retraction element 864 for facilitating retracting outer sheath 842.

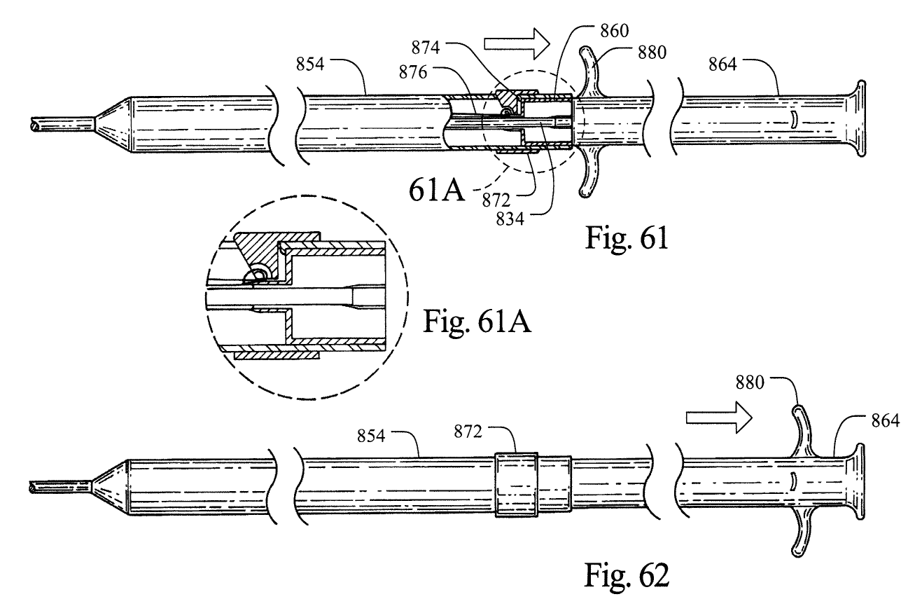

With outer sheath 842 retracted, graft 114 is deployed. In one example, a graft retraction element 872 is coupled to graft 114 and configured to retain graft 114 in a compressed delivery configuration. A graft locking element 874 is formed in or integrated with housing 854. Graft locking element 874 is movable between a locked position, as shown in FIG. 59, and an unlocked position, as shown in FIG. 61. In the locked position, graft locking element 874 extends from an outer surface of housing 854 to interfere with graft retraction element 872 and prevent or limit movement of graft retraction element 872 along axis 856. Graft locking element 874 is moved inwardly with respect to axis 856 to the unlocked position for facilitating deploying graft 114. As shown in FIG. 61, a release string 876 is coupled between graft retraction element 872 and graft 114 such that as graft retraction element 872 is slide along housing 854, release string 876 is uncoupled from graft 114 to release graft 114 for deployment.

In one example, an inner sheath retraction element 880 is movably mounted to handle 852 and coupled to inner sheath 834. Inner sheath retraction element 880 is movable along axis 856 and configured to retract inner sheath 834. As inner sheath retraction element 880 is moved in a distal direction along axis 856, inner sheath 834 is retracted and support stent 126 is released for deployment.

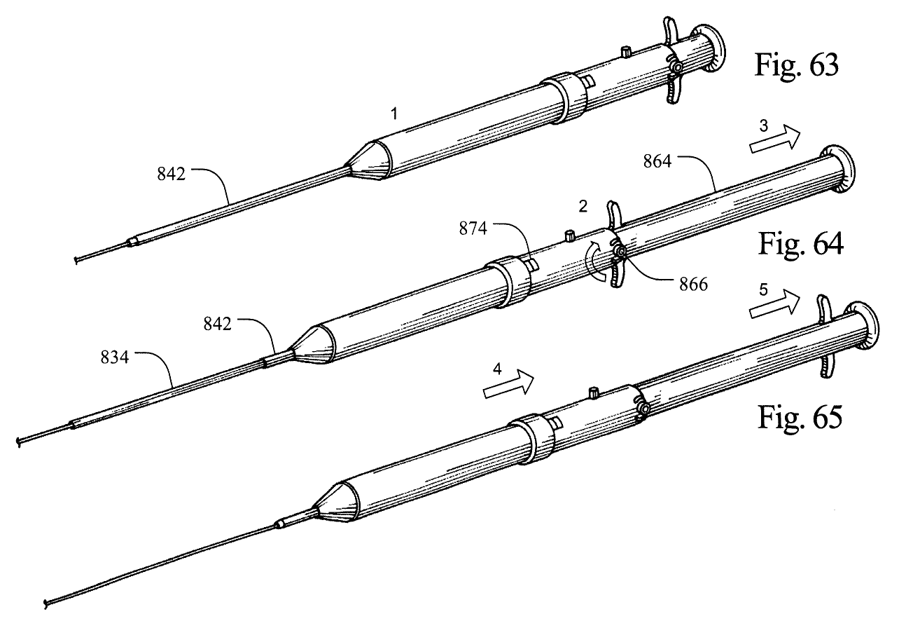

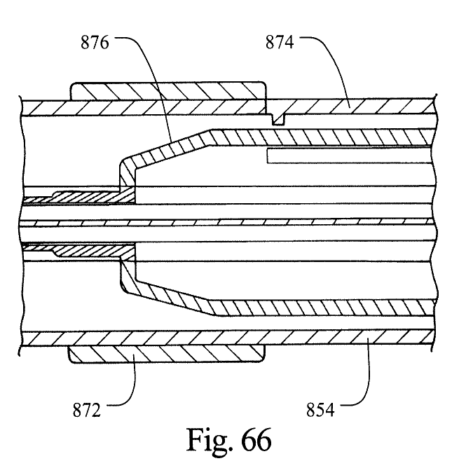

Referring further to FIGS. 63-69, with stent graft 110 properly positioned at the lesion site, outer sheath retraction tube 860, concentrically positioned within housing 854, is unlocked. In this example, outer sheath locking element 866 is rotated to the unlocked position, as shown in FIG. 64. Outer sheath retraction element 864 is pulled in a distal direction along axis 856 to move outer sheath retraction tube 860 within housing 854 and retract outer sheath 842 to expose graft 114. Graft 114 is properly positioned within the vessel at the lesion site and deployed. In one example, graft retraction element 872 is coupled to graft 114 and configured to retain graft 114 in a compressed delivery configuration. Graft locking element 874 is moved from the locked position, as shown in FIG. 63, to the unlocked position, as shown in FIGS. 64 and 66, for facilitating deploying graft 114. Referring further to FIG. 66, release string 876 is uncoupled from graft 114 to release graft 114 for deployment as graft retraction element 872 is slid along housing 854. Inner sheath retraction element 880 is movable in the distal direction along axis 856 to retract inner sheath 834 and release support stent 126 for deployment, as shown in FIG. 65.

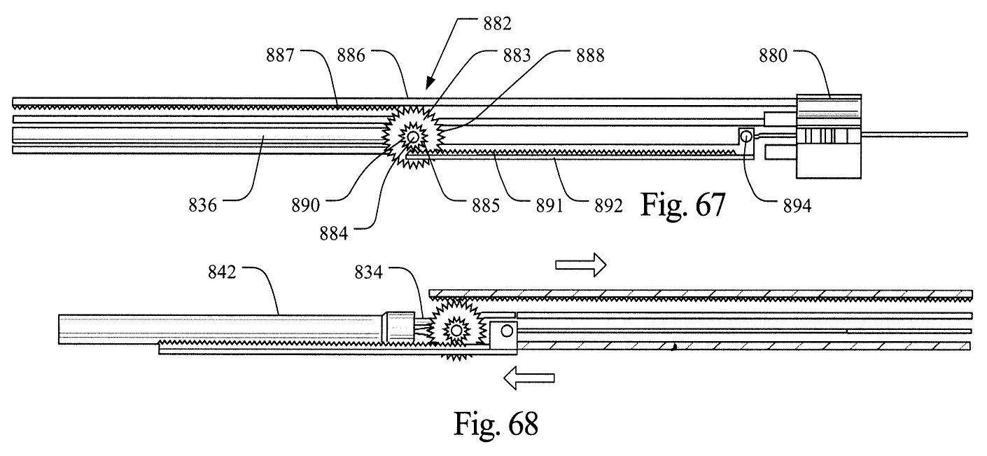

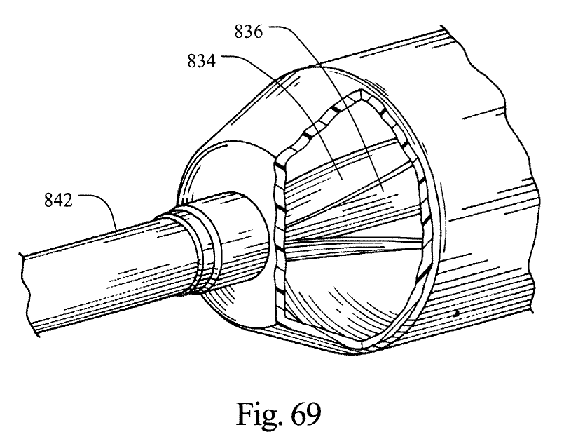

Referring further to FIGS. 67 and 68, inner sheath retraction element 880 is retracted to activate a gear assembly 882 that advances support member 836 as inner sheath retraction element 880 is retracted. In this example, gear assembly 882 is mounted to housing 854. As shown in FIGS. 67 and 68, a first gear 883 is rotatably mounted about an axis 884 and a reduction gear 885 is coupled to first gear 883 and coaxially mounted about axis 884. As inner sheath retraction element 880 is drawn or pulled in the distal direction from an initial position, as shown in FIG. 67, to a final position, as shown in FIG. 68, a rack 886 forming a plurality of teeth 887 cooperates with corresponding teeth 888 formed about a periphery of first gear 883 to rotate first gear 883 about axis 884. As first gear 883 rotates about axis 884, reduction gear 885 coupled to first gear 883 also rotates about axis 884. As shown in FIGS. 67 and 68, reduction gear 885 forms a plurality of teeth 890 about a periphery of reduction gear 885 that cooperate with a plurality of teeth 891 formed on a rack 892. Rack 892 is coupled to support member 836 at bracket 894. Referring to FIGS. 67 and 68, as inner sheath retraction element 880 is drawn or pulled in the distal direction to retract inner sheath 834, gear assembly 882 advances support member 836 in an opposing proximal direction to contact support stent 126 and maintain support stent 126 properly positioned at the lesion site. As shown in FIG. 69, sheath 834 is slotted to accommodate pins and/or support members of gear assembly 882. Inner sheath 834 is coupled to inner sheath retraction element 880 using an interference grip, as shown in FIG. 67, or any suitable fitting mechanism. Further, outer sheath retraction tube 860 is slotted to accommodate pins and/or support members of inner sheath retraction element 880. In this example, outer sheath 842 is coupled to outer retraction tube 860 using a barb fitting, as shown in FIG. 69, or other suitable fitting.

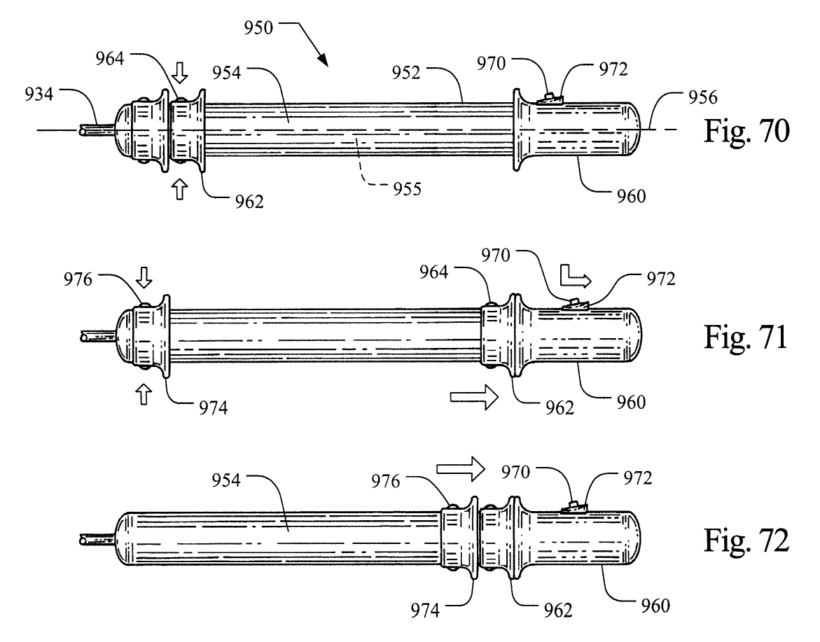

Referring to FIGS. 70-80, in one example an actuator 950 may include a handle 952 operatively coupled to inner sheath 934 and/or outer sheath 942. Handle 952 may include a housing 954 defining a chamber 955 along at least a portion of a length of housing 954 and an axis 956. A housing grip 960 is coupled to a distal end of housing 954, as shown in FIGS. 70-72. An outer sheath retraction element 962 is positioned about housing 954 and coupled to outer sheath 942. Outer sheath retraction element 962 is slidably movable along housing 954 with respect to axis 956 between a proximal end of housing 954 and housing grip 960 to retract outer sheath 942. In one example, at least one locking element 964 is coupled to or positioned with respect to outer sheath retraction element 962 and configured to retain outer sheath retraction element 962 in a locked position, as shown in FIG. 70. With outer sheath retraction element 962 in the locked position, movement of outer sheath 942 with respect to axis 956 is prevented or limited. By pressing cooperating locking element 964, outer sheath retraction element 962 is released to an unlocked position for facilitating retracting outer sheath 942. In one example, in the unlocked position outer sheath retraction element 962 is movable in a distal direction along axis 956 to retract outer sheath 942 and expose graft 114.

With outer sheath 942 retracted, graft 114 is deployed. In one example, a graft release locking element 970 is mounted to housing grip 960 and is configured to control and/or activate release and/or deployment of graft 114. A graft retraction element 972 is operatively coupled to graft release locking element 970. Further, graft retraction element 972 is operatively coupled to graft 114. Movement of graft retraction element 972 initiates deployment of graft 114. Referring to FIG. 70, graft release locking element 970 initially retains graft retraction element 972 in a locked position and graft 114 in a delivery configuration. Graft release locking element 970 is movable between a biased position and a release position, such as pressing graft release locking element 970, to move graft retraction element 972 to an unlocked position, as shown in FIG. 71. In the unlocked position, graft retraction element 972 is slidably movable with respect to housing grip 960 for facilitating deploying graft 114.

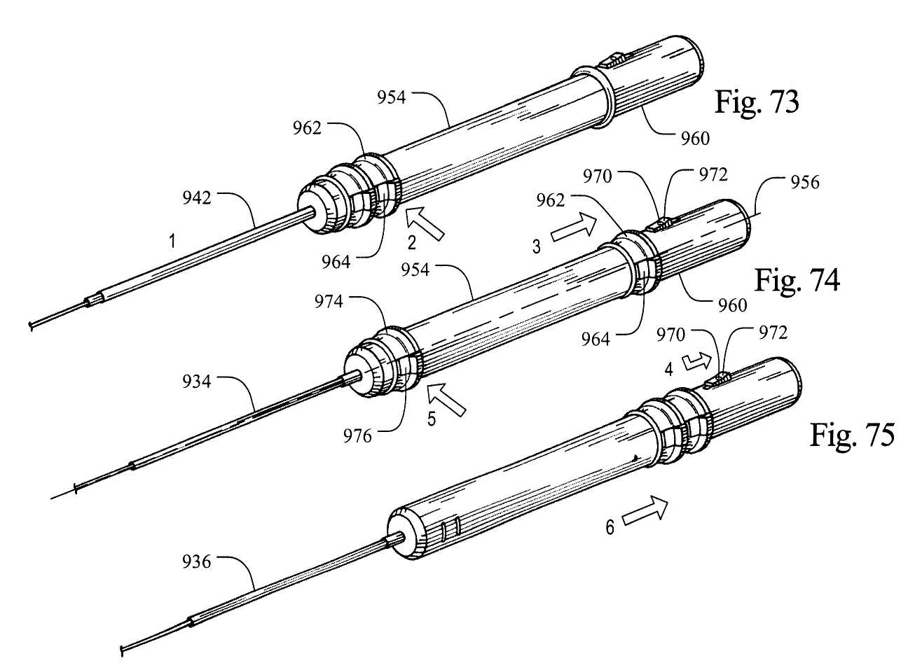

In one example, an inner sheath retraction element 974 is positioned about housing 954 and coupled to inner sheath 934. Inner sheath retraction element 974 is slidably movable along housing 954 with respect to axis 956 between a proximal end of housing 954 and outer sheath retraction element 962 to retract inner sheath 934, as shown in FIG. 72. In this example, a locking element 976 is coupled to or positioned with respect to inner sheath retraction element 974 and configured to retain inner sheath retraction element 974 in a locked position, as shown in FIG. 71. With inner sheath retraction element 974 in the locked position, movement of inner sheath 934 with respect to axis 956 is prevented or limited. By pressing locking element 976, inner sheath retraction element 974 is released to an unlocked position for facilitating retracting inner sheath 934. In one example, in the unlocked position inner sheath retraction element 974 is movable in a distal direction along axis 956 to retract inner sheath 934 and release and/or deploy support stent 126, as shown in FIG. 72.

Referring further to FIGS. 73-80, with stent graft 110 properly positioned at the lesion site, outer sheath retraction element 962, positioned about housing 954 and coupled to outer sheath 942, is unlocked by pressing locking element 964 to release outer sheath retraction element 962, as shown in FIG. 73. As shown in FIG. 74, outer sheath retraction element 962 is movable in the distal direction along housing 954 with respect to axis 956 between the proximal end of housing 954 and housing grip 960 to retract outer sheath 942 and expose graft 114.

With outer sheath 942 retracted, graft 114 positioned within the vessel at the lesion site is deployed. Graft release locking element 970 is movable between the biased position and the release position, such as pressing graft release locking element 970, to move graft retraction element 972 to an unlocked position, as shown in FIG. 75. In the unlocked position, graft retraction element 972 is slidably movable with respect to housing grip 960 to deploy graft 114.

After graft 114 is deployed, inner sheath 934 is retracted to deploy support stent 126. As shown in FIG. 74, in one example, inner sheath retraction element 974 is unlocked by pressing locking element 976, which is movable between the locked position and the unlocked position. In the locked position, locking element 976 is configured to limit movement of inner sheath 934 with respect to axis 956. Inner sheath retraction element 974 is moved along housing 954 between a proximal end of housing 954 and outer sheath retraction element 962, as shown in FIG. 75, to retract inner sheath 934 and deploy support stent 126.

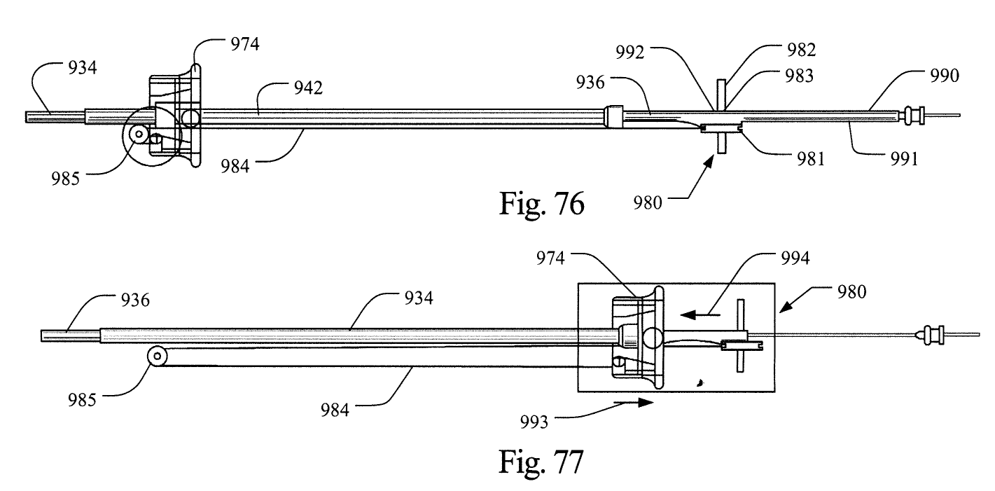

Referring further to FIGS. 76 and 77, inner sheath retraction element 974 is retracted to activate a rack and pinion assembly 980 that advances support member 936 as inner sheath retraction element 974 is moved in the distal direction along housing 954. In this example, rack and pinion assembly 980 may include a pulley 981 rotatable mounted about a shaft 982 that is mounted to housing 954. As shown in FIGS. 76 and 77, a pinion 983 is coupled to pulley 981 and coaxially mounted about shaft 982. A string 984 is coupled at a first end to a distal end of inner sheath 934 and extends and wraps around pulley 981 of rack and pinion assembly 980. String 984 extends in a proximal direction with respect to rack and pinion assembly 980 through inner sheath retraction element 974 to wrap about a second pulley 985 rotatably mounted to housing 954 proximal to inner sheath retraction element 974. As shown in FIGS. 76 and 77, string 984 wraps around second pulley 985 and is coupled at a second end to inner sheath retraction element 974. In this example, a support member 936 may include a rack portion 990 forming a plurality of teeth 991 that cooperate with corresponding teeth 992 formed about a periphery of pinion 983. As inner sheath retraction element 974 is drawn or pulled in the distal direction as shown by directional arrow 993, from an initial position as shown in FIG. 76 to a final position as shown in FIG. 77, string 984 is drawn or pulled in the distal direction Pulley 981 rotates as inner sheath 934 is retracted. Pinion 983 coupled to pulley 981 also rotates such that teeth 992 formed on the periphery of pinion 983 cooperate with corresponding teeth 991 formed on rack portion 990 to advance support member 936 in an opposing proximal direction as shown by directional arrow 994 in FIG. 77. Support member 936 advances to contact support stent 126 and maintain support stent 126 properly positioned at the lesion site.

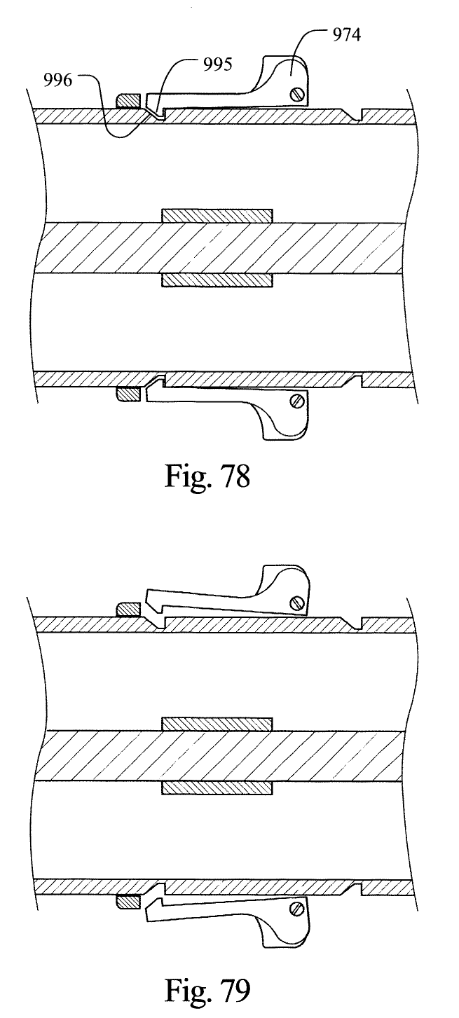

As shown in FIG. 78, locking elements 976 are pivotally coupled to inner sheath retraction element 974 such that with inner sheath retraction element 974 in the locked position a snap component 995 formed on locking elements 976 are positioned within a corresponding depression 996 defined in housing 954. By pressing locking elements 976, snap component 995 is released from within corresponding depression 996 and inner sheath retraction element 974 is released to an unlocked position for facilitating retracting inner sheath 934, as shown in FIG. 79.

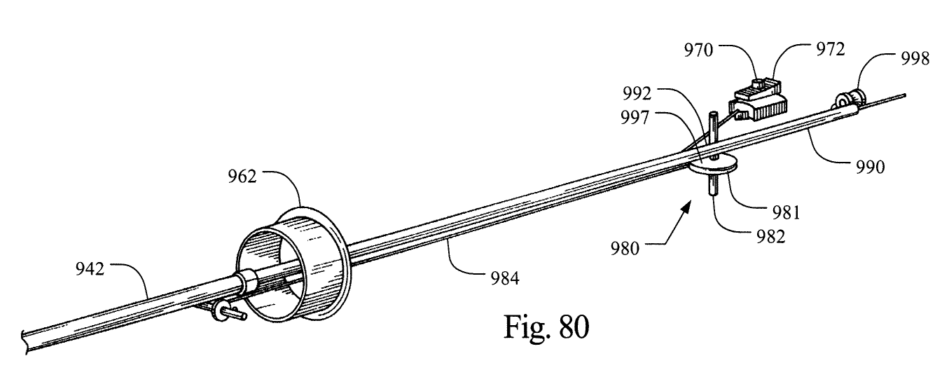

A string 997 may be coupled at a first end to graft retraction element 972, as shown in FIG. 80. An opposing second end of string 997 is coupled about graft 114 (not shown) using at least one slip knot or other suitable coupling mechanism or technique. As graft retraction element 972 is pulled, string 997 is pulled to release the slip knot coupled about graft 114 to release graft 114, which is then deployed to a deployed position at the lesion site. A luer lock fitting 998 may be positioned with respect to chamber 955 defined within housing 954 for facilitating sufficient irrigation during the procedure.

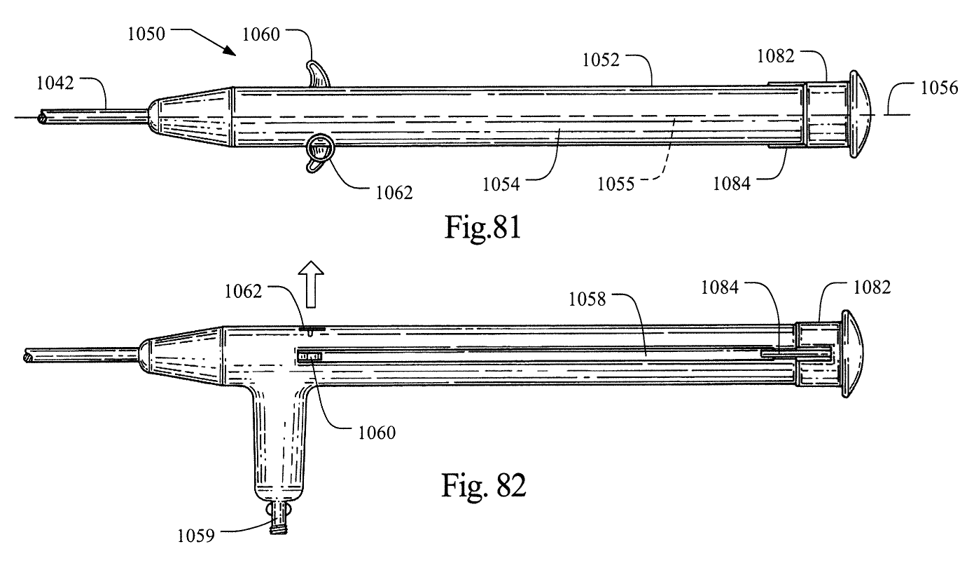

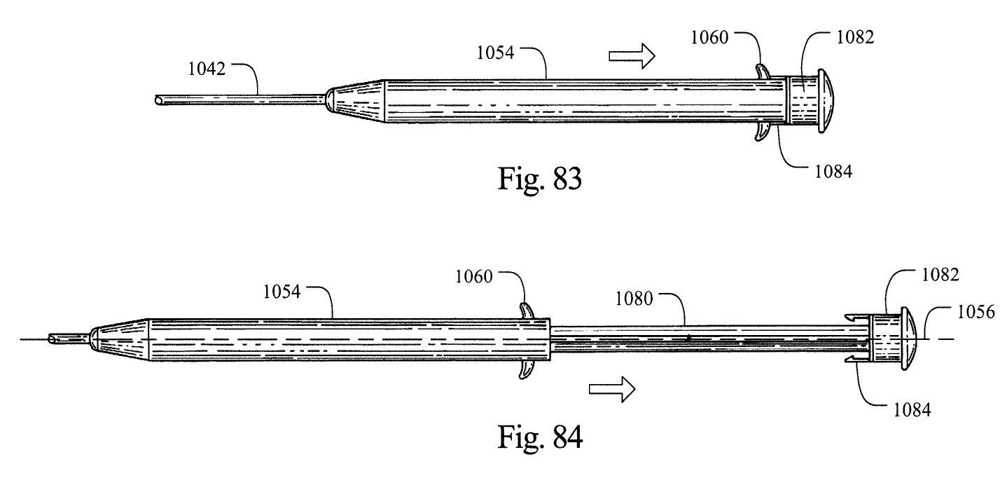

Referring to FIGS. 81-90, an actuator 1050 may include a handle 1052 operatively coupled to inner sheath 1034 and/or outer sheath 1042. Handle 1052 may include a housing 1054 defining a chamber 1055 along at least a portion of a length of housing 1054 and an axis 1056. As shown in FIG. 82, housing 1054 further defines a track 1058 in communication with at least a portion of chamber 1055. In one example, handle 1052 may include an irrigation tube 1059 coupled to or integrated with handle 1052 and in fluid communication with the vessel for facilitating irrigating undesirable fluids and/or air from within the vessel during the procedure.

An outer sheath retraction element 1060 is coupled to outer sheath 1042 and at least partially positioned within track 1058. Outer sheath retraction element 1060 is movable within track 1058 and configured to retract outer sheath 1042. A locking element 1062 is positionable within housing 1054 and configured to lock outer sheath retraction element 1060 to prevent or limit movement of outer sheath retraction element 1060 within track 1058.

An inner sheath retraction tube 1080 is movably positioned at least partially within chamber 1055 and coupled to inner sheath 1034. Referring to FIG. 84, inner sheath retraction tube 1080 is movable within chamber 1055 along axis 1056 for facilitating retracting inner sheath 1034. In one example, a retraction element 1082 is coupled to or integrated with inner sheath retraction tube 1080. Retraction element 1082 is initially coupled to a distal end of housing 1054, as shown in FIGS. 81-83, to retain inner sheath retraction tube 1080 in a locked position to prevent or limit undesirable movement of inner sheath 1034. Retraction element 1082 may include at least one finger 1084 that is initially coupled to the distal end of housing 1054. As shown in FIG. 82, finger 1084 is initially positioned within a corresponding track 1058 to couple retraction element 1082 to housing 1054. As outer sheath retraction element 1060 is moved in the distal direction with respect to axis 1056, outer sheath retraction element 1060 contacts fingers 1084 coupling retraction element 1082 to housing 1054. Such contact unlocks inner sheath retraction tube 1080, which is then moved in the distal direction with respect housing 1054 along axis 1056 to retract inner sheath 1034 and release and/or deploy support stent 126.

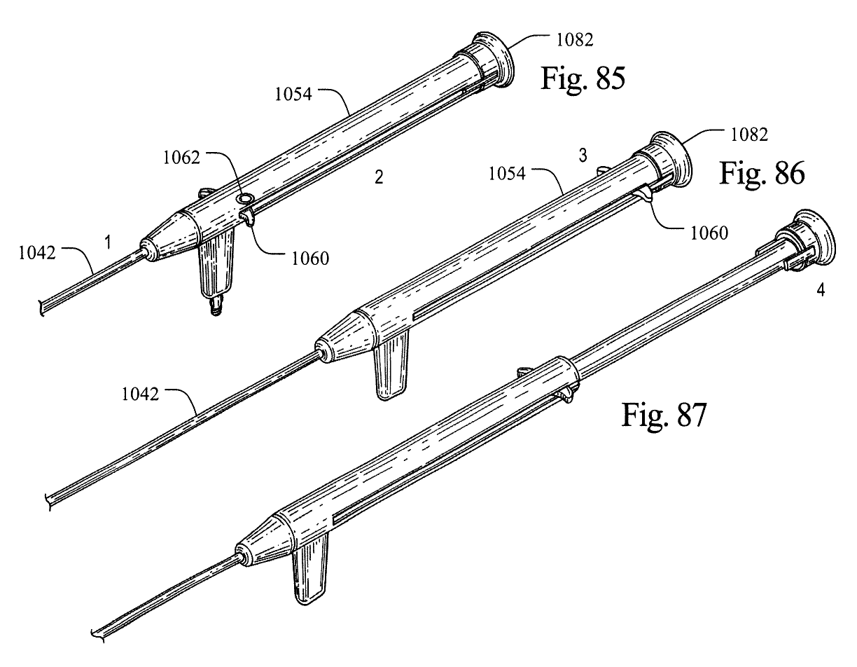

Referring to FIGS. 85-90, with stent graft 110 properly positioned at the lesion site, the delivery system is unlocked by removing locking element 1062 from within housing 1054. Locking element 1062 is initially coupled through housing 1054 to outer sheath retraction element 1060 and is configured to retain outer sheath 1042 and inner sheath 1034 in a delivery configuration, as shown in FIG. 85. As shown in FIG. 86, outer sheath retraction element 1060 is movable in the distal direction along housing 1054 with respect to axis 1056 between the proximal end of housing 1054 and retraction element 1082 coupled to the distal end of housing 1054 to retract outer sheath 1042 and automatically release graft 114. As outer sheath retraction element 1060 is moved along housing 1054, outer sheath retraction element 1060 contacts retraction element 1082 to decouple retraction element 1082 from housing 1054. As shown in FIG. 87, retraction element 1082 is then moved along axis 1056 in a distal direction to retract inner sheath 1034 and release and/or deploy support stent 126.

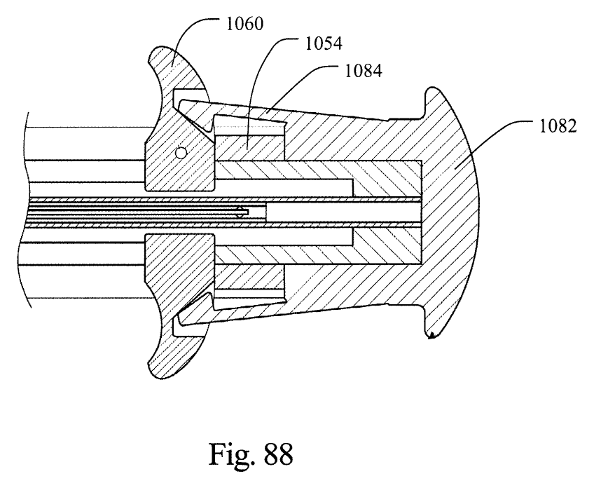

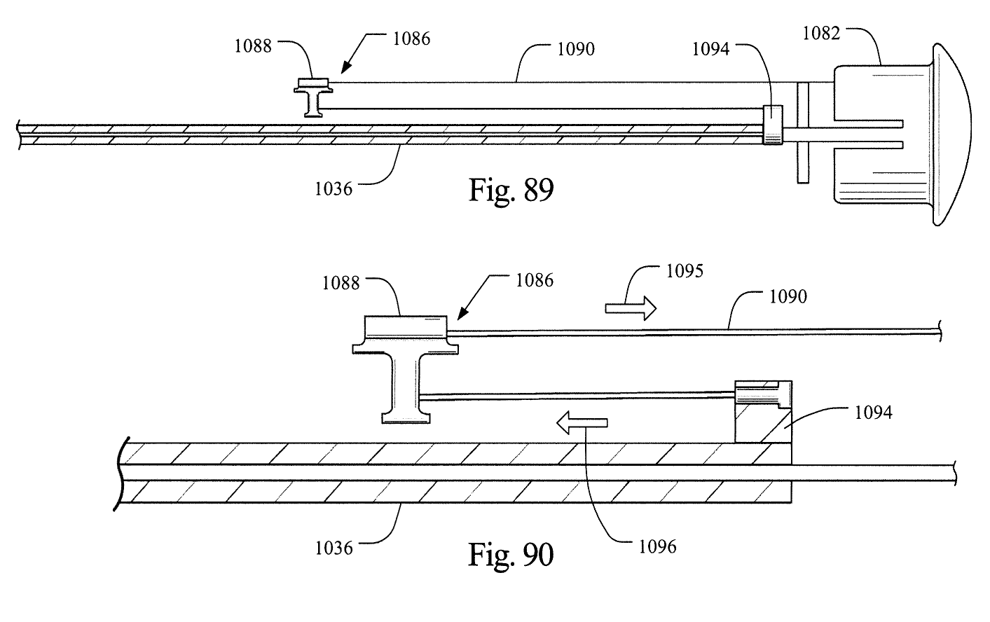

Referring further to FIGS. 88-90, with outer sheath retraction element 1060 in a retracted position, outer sheath retraction element 1060 contacts fingers 1084 to unlock fingers 1084 from housing 1054 and decouple retraction element 1082 from housing 1054. Inner sheath retraction tube 1080 is then moved in the distal direction with respect housing 1054 along axis 1056 to retract inner sheath 1034 and release and/or deploy support stent 126. As shown in FIGS. 89 and 90, retraction element 1082 is retracted to activate a spindle arrangement 1086 for facilitating advancing support member 1036 as inner sheath retraction tube 1080 is moved in the distal direction along housing 1054. In this example, spindle arrangement 1086 may include a spindle 1088 rotatably mounted to housing 1054. A string 1090 is coupled at a first end to retraction element 1082 and extends in the proximal direction to wrap around and/or through spindle 1088. String 1090 extends in the distal direction with respect to spindle 1088 and is coupled at an opposing second end to support member 1036. In one example, support member 1036 may include a block 1094 to which string 1090 is coupled. In this example, as retraction element 1082, and inner sheath retraction tube 1080 coupled thereto, is drawn in the distal direction as shown by directional arrow 1095, from an initial position as shown in FIG. 89 to a final position as shown in FIG. 90, string 1090 is drawn or pulled in the distal direction, which causes support member 1036 to advance in an opposing proximal direction as shown by directional arrow 1096. String 1090 moves about spindle 1088 causing spindle 1088 to rotate for facilitating smooth retraction of inner sheath 1034 and accurate deployment of support stent 126 at the lesion site.

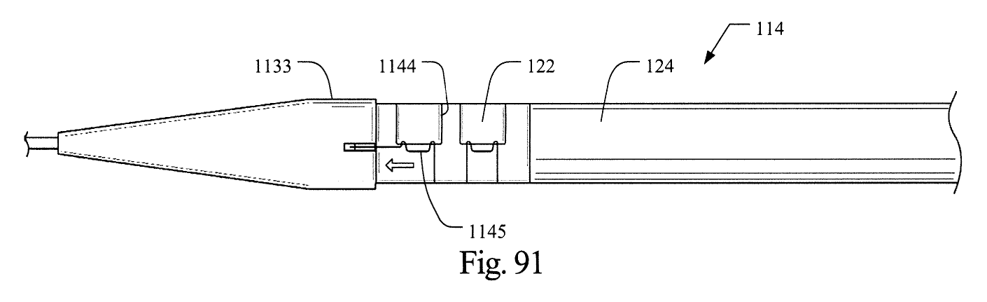

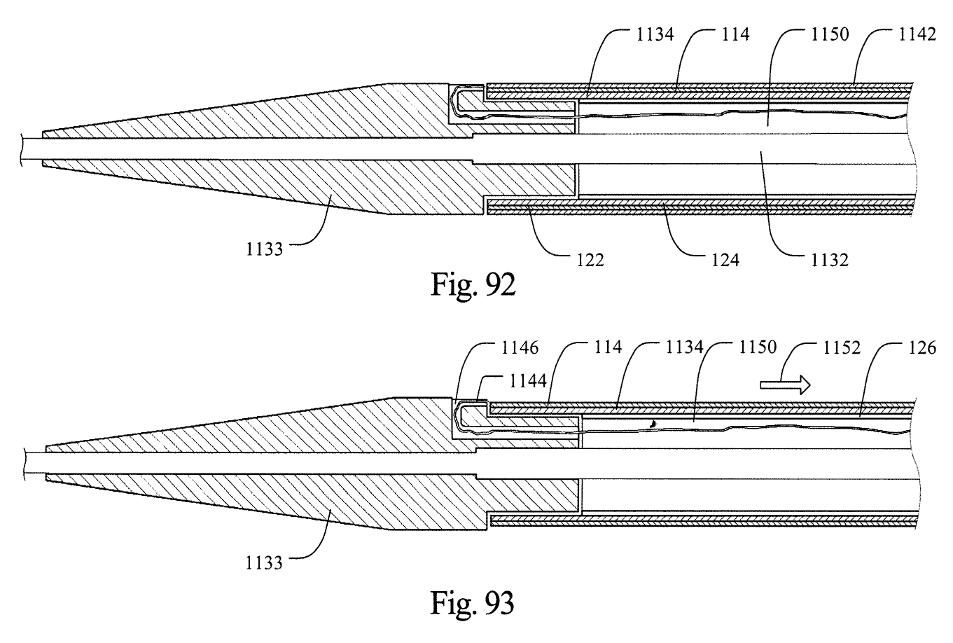

Referring now to FIGS. 91-93, during a thoracic aortic aneurysm repair procedure, a delivery system 1130 delivers and/or positions stent graft 110 with respect to the lesion site at or near the aneurysm. As shown in FIGS. 92 and 93, graft 114 is slidably positioned about inner sheath 1134. A portion of inner sheath 1134 is coupled to nose cone 1133. Outer sheath 1142 is retractably positioned about graft 114 with graft 114 in the delivery configuration to maintain graft 114 in the delivery configuration as stent graft 110 is advanced to the lesion site. With stent graft 110 positioned within the vessel as desired, outer sheath 1142 is retractable for facilitating deployment of graft 114 from the delivery configuration to the deployed configuration.

In one example, a string 1144 is positioned about at least a portion of graft 114, such as graft portion 122, and configured to temporarily maintain graft 114 in the compressed delivery configuration after outer sheath 1142 is retracted from about graft 114. As shown in FIG. 91, string 1144 may include at least one slip knot 1145 to maintain graft 114 in the compressed delivery configuration. String 1144 is fed through a passage 1146 formed in nose cone 1133 and into passage 1150 defined between an inner surface of support stent 126 and an outer surface of wire lumen 1132. String 1100 is coupled to an actuator, such as described above, that is configured to pull or drawn string 1100 to release graft 114, which then expands from the delivery configuration to the deployed configuration.

With delivery system 1130 at the lesion site, outer sheath 1142 is moved in a distal direction, as shown by directional arrow 1152 in FIG. 93, to retract outer sheath 1142 and expose at least a portion of graft 114. The actuator is activated to release string 1144 from about graft 114 and graft 114 expands in a radial direction with respect to wire lumen 1132 between the delivery configuration and the deployed configuration. In the deployed configuration, an outer radial surface of graft 114 contacts the interior surface of the vessel wall at the lesion site and graft 114 defines a passage therethrough. Proximal end 118 of graft 114 is positioned proximal to the aneurysm and distal end 120 is positioned distal to the aneurysm.

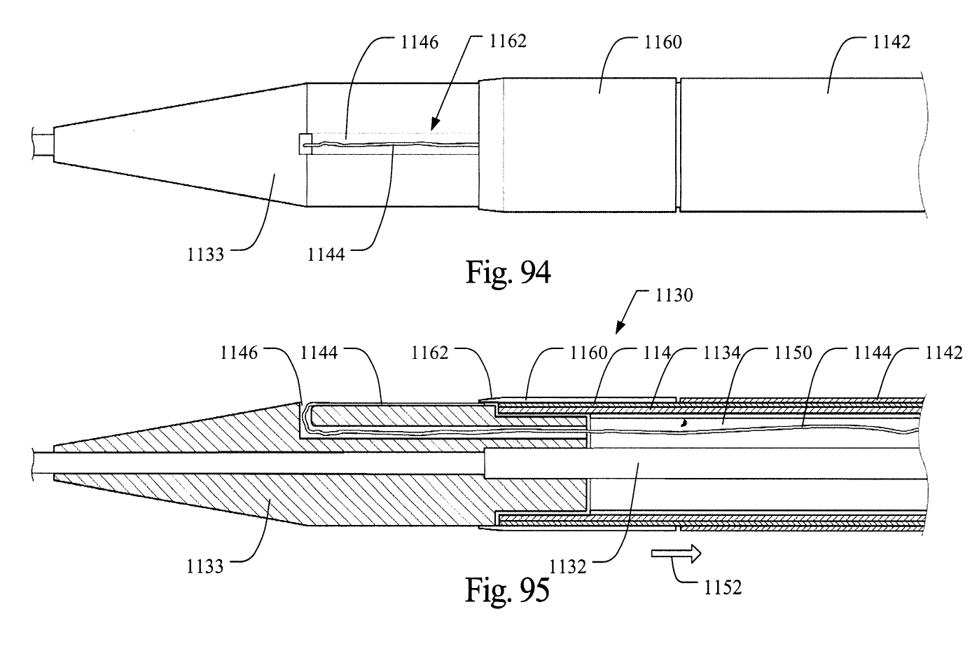

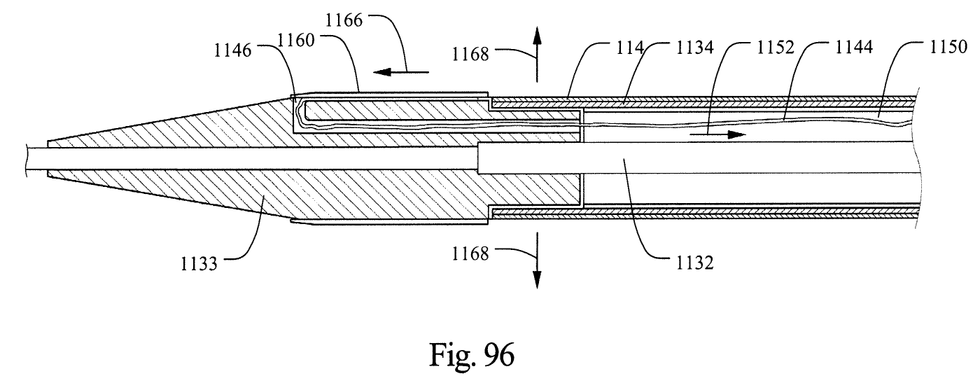

Alternatively, as shown in FIGS. 94-96, string 1144 is coupled to a retaining ring 1160 positioned about at least a portion of graft 114, such as graft portion 122. Retaining ring 1160 is slidably movable with respect to nose cone 1133 between an initial position and a release position. In the initial position, retaining ring 1160 is configured to temporarily maintain graft 114 in the compressed delivery configuration after outer sheath 1142 is retracted from about graft 114, as shown in FIGS. 94 and 95. In the release position, as shown in FIG. 96, retaining ring is configured for facilitating deployment of graft 114. String 1144 is coupled at a first end 1162 to retaining ring 1160 and fed through passage 1146 defined by nose cone 1133, as shown in FIG. 94, and into passage 1150 defined between an inner surface of support stent 126 and an outer surface of wire lumen 1132, as shown in FIGS. 95 and 96. A second end 1164 of string 1144 is coupled to an actuator, such as described above, that is configured to pull or draw string 1144 in the distal direction, as shown by directional arrow 1152 in FIG. 96, and move retaining ring 1160 in an opposing proximal direction, as shown by directional arrow 1166 in FIG. 96, to release graft 114. Released graft 114 expands in a radial direction, as shown by directional arrows 1168 in FIG. 96, from the delivery configuration to the deployed configuration.

With delivery system 1130 at the lesion site, outer sheath 1142 is moved in the distal direction, as shown by directional arrow 1152 in FIG. 95, to retract outer sheath 1142 and expose at least a portion of graft 114. The actuator is activated to pull or draw string 1144 in the distal direction and move retaining ring 1160 in the proximal direction to release graft 114, as shown in FIG. 96. Graft 114 expands in a radial direction with respect to wire lumen 1132 between the delivery configuration and the deployed configuration. In the deployed configuration, an outer radial surface of graft 114 contacts the interior surface of the vessel wall at the lesion site and graft 114 defines a passage therethrough. Proximal end 118 of graft 114 is positioned proximal to the aneurysm and distal end 120 is positioned distal to the aneurysm.

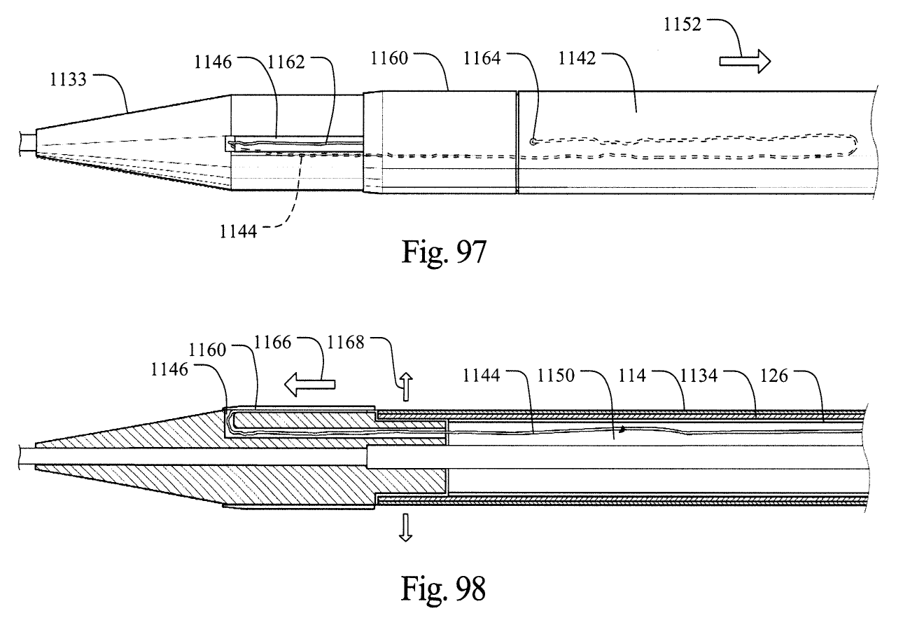

Alternatively, as shown in FIGS. 97 and 98, second end 1164 of string 1144 is coupled to outer sheath 1142. With delivery system 1130 at the lesion site, outer sheath 1142 is moved in the distal direction, as shown by directional arrow 1152 in FIG. 97, to retract outer sheath 1142 and expose at least a portion of graft 114. As outer sheath 1142 is retracted, outer sheath 1142 draws string 1144 in the distal direction and moves retaining ring 1160 in the proximal direction to release graft 114, as shown in FIG. 98. Graft 114 expands in a radial direction with respect to wire lumen 1132 between the delivery configuration and the deployed configuration.

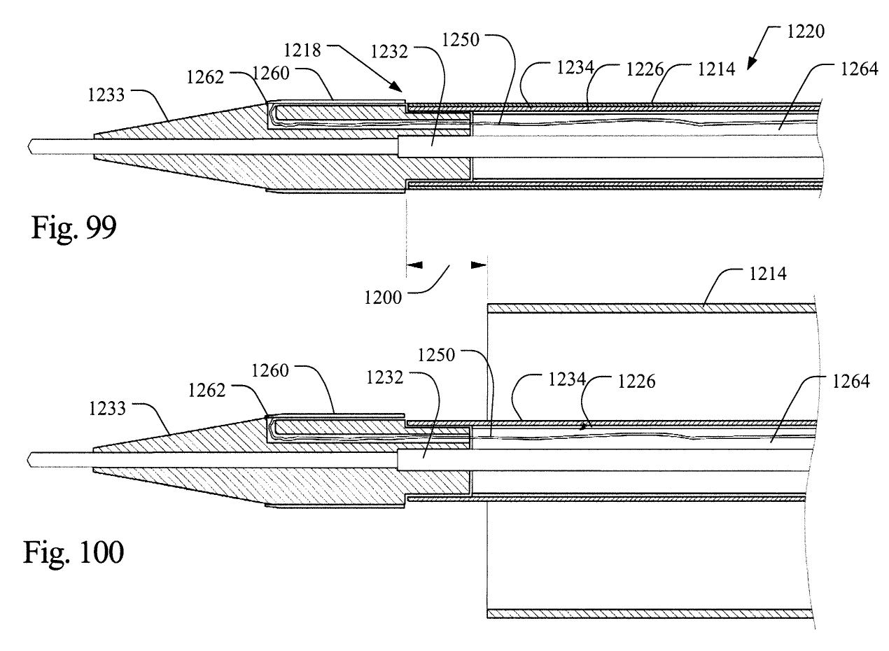

Referring to FIGS. 99-103, graft 114 may be deployed in two stages to prevent undesirable axial migration of graft 114 upon deployment. For example, referring further to FIGS. 99 and 100, the aorta has a diameter of about 1.12 inches and a cross-section area of about 0.1284 in². Blood flows through the aorta at a velocity of about 12.99 in/sec. As a result, upon deployment of graft 114, graft 114 will be displaced in a distal direction a distance 1200, as shown in FIG. 99, based on several parameters including, without limitation, blood flow rate, dimensions of the aorta section, resisting surface area and/or deployment time.

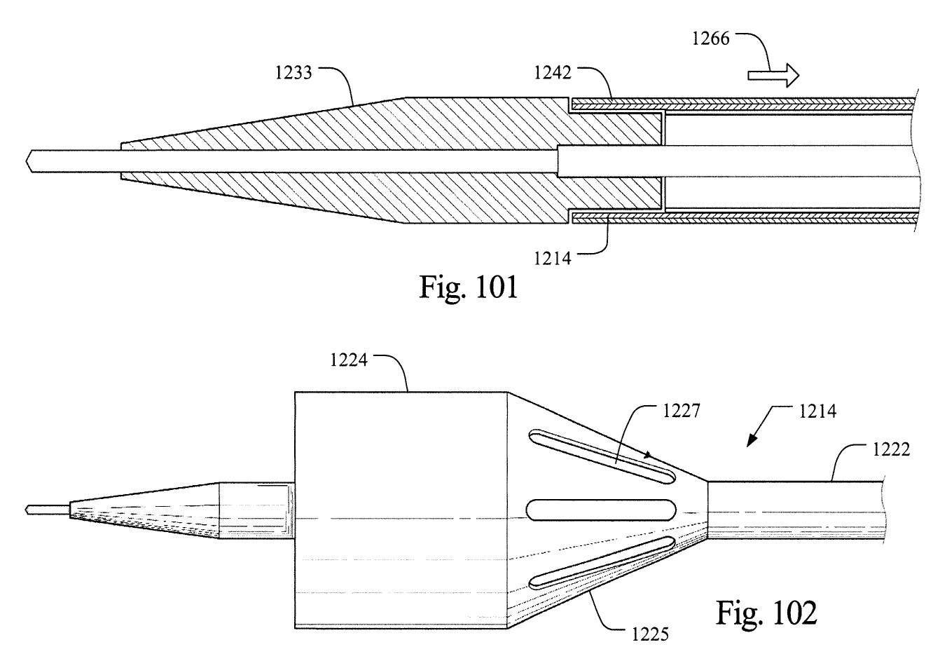

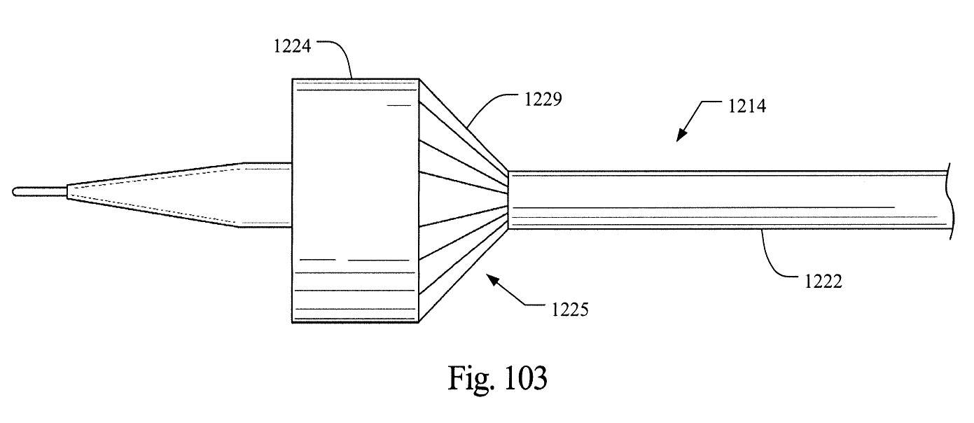

Referring now to FIGS. 101-103, a graft 1214 is deployed in two stages to prevent undesirable axial migration of graft 1214 upon deployment. In a first stage, outer sheath 1242 is retracted a first distance, such as about 1.0 inch to about 2.0 inches, for facilitating partial deployment of graft 1214 to increase the accuracy of placement of graft 1214 without a graft portion 1222 free to migrate. During the first stage, an anchor portion 1224 of graft 1214 is deployed, as shown in FIG. 102. Upon deployment of anchor portion 1224, outer sheath 1242 is retracted during a second stage to deploy the remaining portion of graft 1214. During the second stage, a speed at which outer sheath 1242 is retracted is substantially equal to a blood flow rate through the vessel to minimize pressure on graft 1214 during deployment. Graft 1214 may include a transition portion 1225 coupling anchor portion 1224 to graft portion 1222. Transition portion 1225 defines a plurality of perforations 1227, as shown in FIG. 102, for facilitating blood flow through graft 1214 as graft 1214 expands to engage the inner wall of the aorta. Alternatively, transition portion 1225 may include a plurality of strings 1229 that couple anchor portion 1224 to graft portion 1222, as shown in FIG. 103, for facilitating blood flow through graft 1214 as graft 1214 expands.

For example, referring further to FIGS. 99 and 100, a string 1250 is coupled to a retaining ring 1260 positioned about at least a portion of graft 1214, such as graft portion 122. Retaining ring 1260 is slidably movable with respect to nose cone 1233 between an initial position configured to temporarily maintain graft 1214 in the compressed delivery configuration after outer sheath 1242 is retracted from about graft 1214, as shown in FIG. 99, and a release position, as shown in FIG. 100, for facilitating deployment of graft 1214. String 1250 is coupled at a first end to retaining ring 1260 and fed through passage 1262 defined by nose cone 1233, as shown in FIGS. 99 and 100, and into passage 1264 defined between an inner surface of support stent 1226 and an outer surface of wire lumen 1232, as shown in FIGS. 99 and 100. A second end (not shown) of string 1250 is coupled to an actuator, such as described above, that is configured to draw string 1250 in the distal direction, as shown by directional arrow 1266 in FIG. 101, and move retaining ring 1260 in an opposing proximal direction, to release graft 1214. Released graft 1214 expands in a radial direction, from the delivery configuration to the deployed configuration.

With delivery system 1230 at the lesion site, outer sheath 1242 is moved in the distal direction, to retract outer sheath 1242 and expose at least a portion of graft 1214. The actuator is activated to pull or draw string 1250 in the distal direction and move retaining ring 860 in the proximal direction to release graft 1214, as shown in FIG. 99. Graft 1214 expands in a radial direction with respect to wire lumen 1232 between the delivery configuration and the deployed configuration. In the deployed configuration, an outer radial surface of graft 1214 contacts the interior surface of the vessel wall at the lesion site and graft 1214 defines a passage therethrough. Proximal end 1218 of graft 1214 is positioned proximal to the aneurysm and distal end 1220 is positioned distal to the aneurysm.

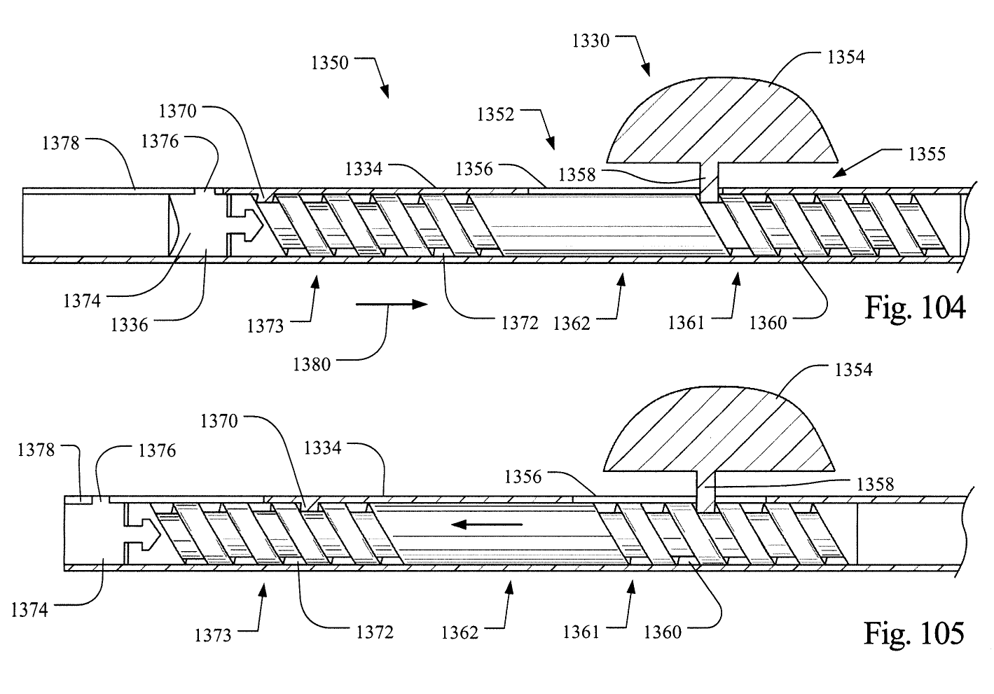

Referring to FIGS. 104 and 105, a delivery system 1330 may include an actuator 1350 having a handle 1352 operatively coupled to inner sheath 1334 and an outer sheath (not shown). Handle 1352 may include a housing 1354 defining a chamber 1355. Inner sheath 1334 is slidably positioned within chamber 1355 and defines a first slot 1356. As shown in FIGS. 104 and 105, a first or stationary projection 1358 formed by housing 1354 extends through slot 1356 and is positioned within a helical groove 1360 at least partially forming a first cam 1361 within a distal portion 1362 of support member 1336. A second projection 1370 formed on an inner surface of inner sheath 1334 is positioned within a helical groove 1372 at least partially forming a second cam 1373 within distal portion 1362. A tip portion 1374 of support member 1336 is coupled to distal portion 1362 and may include or form a key 1376 that extends at least partially into a second slot 1378 defined by inner sheath 1334. Inner sheath 1334 is retracted by moving inner sheath 134 in a distal direction, as shown by directional arrow 1380 in FIG. 104. As inner sheath 134 is moved in the distal direction, second cam 1373 causes projection 1370 to rotationally advance along helical groove 1372 as first cam 1361 advances with respect to stationary projection 1358 formed on housing 1354. Key 1376 positioned within second slot 1378 prevents tip portion 1374 from rotating as tip portion 1374 moves in the proximal direction. In this example, as inner sheath 1334 is retracted in the distal direction, support member 1336 is advanced in the opposing proximal direction to maintain support stent 126 properly positioned at the lesion site and with respect to graft 114.

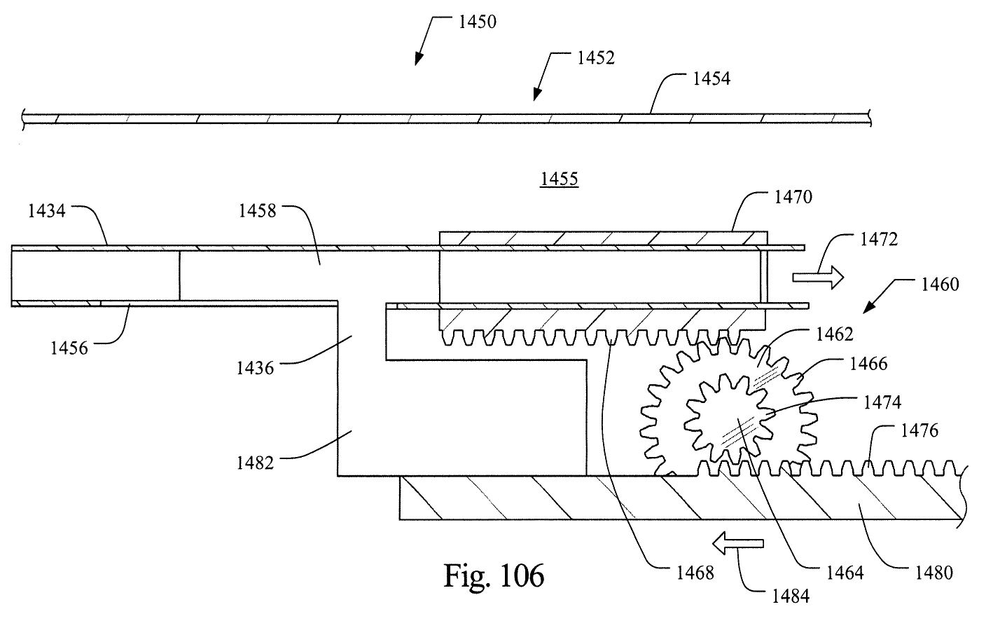

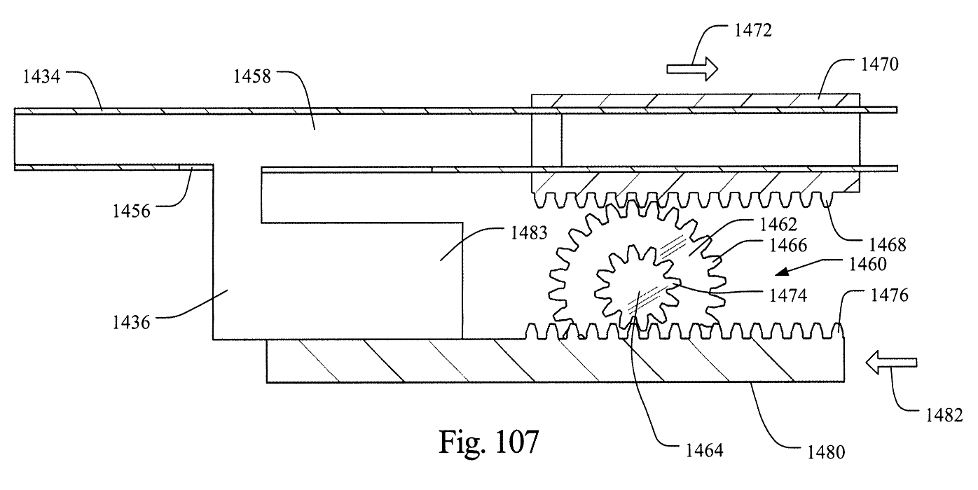

Alternatively, a delivery system 1430 may include an actuator 1450 having a handle 1452 operatively coupled to inner sheath 1434 and an outer sheath (not shown). Handle 1452 may include a housing 1454 defining a chamber 1455. Inner sheath 1434 is slidably positioned within chamber 1455 and defines a first slot 1456. As shown in FIGS. 106 and 107, a first portion 1458 of support member 1436 extends through first slot 1456 and is slidably positioned within inner sheath 1434. A gear assembly 1460 is rotatably mounted within housing 1454 and may include a first gear 1462 and a reduction gear 1464. First gear 1462 forms a plurality of teeth 1466 that cooperate with a plurality of teeth 1468 formed on a rack 1470 coupled to inner sheath 134. As inner sheath 134 is moved in a distal direction, as shown by directional arrow 1472 in FIG. 106, rack 1470 moves with respect to first gear 1462 causing first gear 1462 to rotate as each tooth 1468 cooperates with corresponding teeth 1466 formed on first gear 1462. Simultaneously, reduction gear 1464 rotates and a plurality of teeth 1474 formed on reduction gear 1464 cooperate with a plurality of teeth 1476 formed on a rack 1480 to cause rack 1480 to move in a proximal direction as shown by directional arrow 1482 in FIG. 85-A. Rack 1480 is coupled to a base portion 1483 of support member 1436 and, thus, movement of rack 1480 in the proximal direction results in advancement of support member 1436 within inner sheath 1434.

In this example, inner sheath 1434 is retracted by moving inner sheath 1434 in the distal direction. As inner sheath 1434 moves in the distal direction, rack 1470 moves with respect to first gear 1462 to cause gear assembly 1460 to rotate. As gear assembly 1460 rotates, rack 1480 moves in the proximal direction as shown by directional arrow 1484, causing first portion 1458 of support member 1436 to advance, as shown in FIG. 107. In this example, as inner sheath 1434 is retracted in the distal direction, support member 1436 is advanced in the opposing proximal direction to maintain support stent 126 properly positioned at the lesion site and with respect to graft 114.

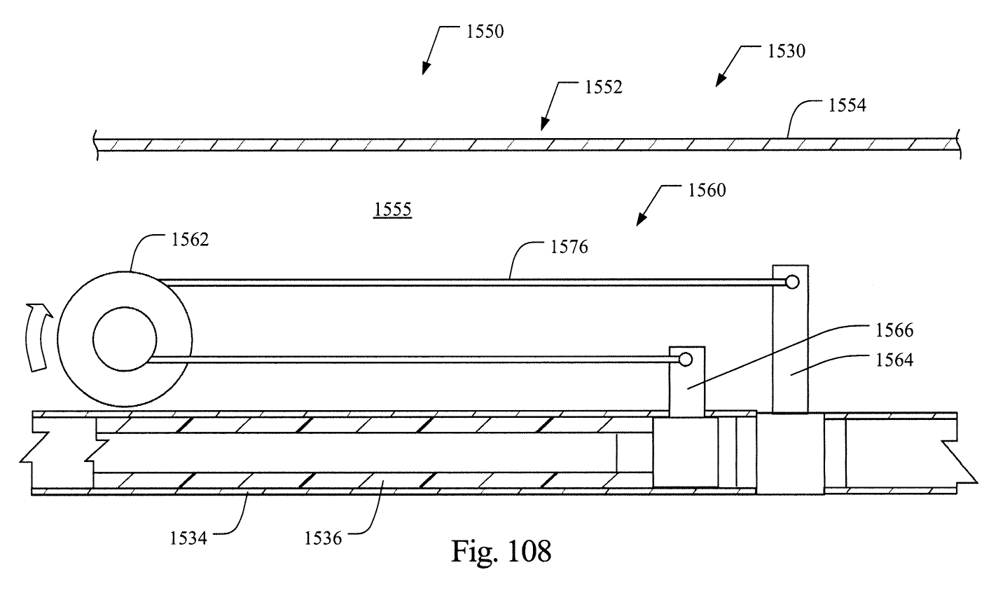

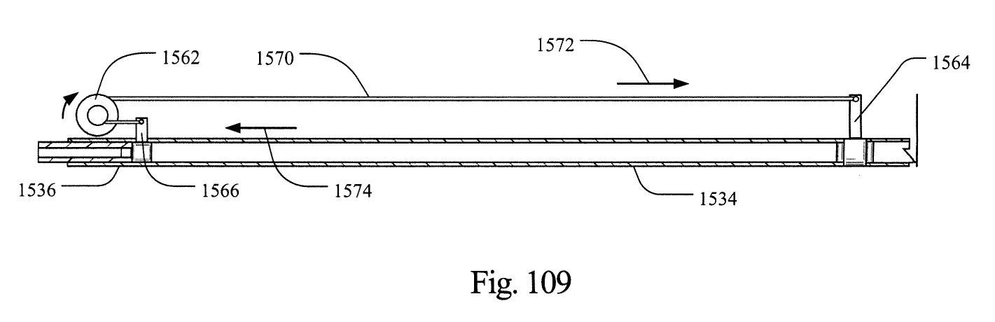

Alternatively, a delivery system 1530 may include an actuator 1550 having a handle 1552 operatively coupled to inner sheath 1434 and an outer sheath (not shown). Handle 1552 may include a housing 1554 defining a chamber 1555. Inner sheath 1534 is slidably positioned within chamber 1555. As shown in FIGS. 108 and 109, a pulley assembly 1560 is positioned within housing 1554. Pulley assembly 1560 may include a hub 1562 rotatably mounted to housing 1554. A first bracket 1564 is coupled to inner sheath 1534 and a second bracket 1566 is positioned within inner sheath 1534 to contact support member 1536. A first end of a string 1570 is coupled to first bracket 1564 and wrapped around hub 1562. An opposing second end of string 1570 is coupled to second bracket 1566. As inner sheath 1534 is moved in a distal direction, as shown by directional arrow 1572 in FIG. 109, first bracket 1564, coupled to inner sheath 1534, also moves in the distal direction, which causes hub 1562 to rotate and draw second bracket 1566 in an opposing proximal direction, as shown by directional arrow 1574 in FIG. 109. As second bracket 1566 moves in the proximal direction, second bracket 1566 contacts support member 1536 and urges support member 1536 to advance in the proximal direction to maintain support stent 126 properly positioned at the lesion site and with respect to graft 114.

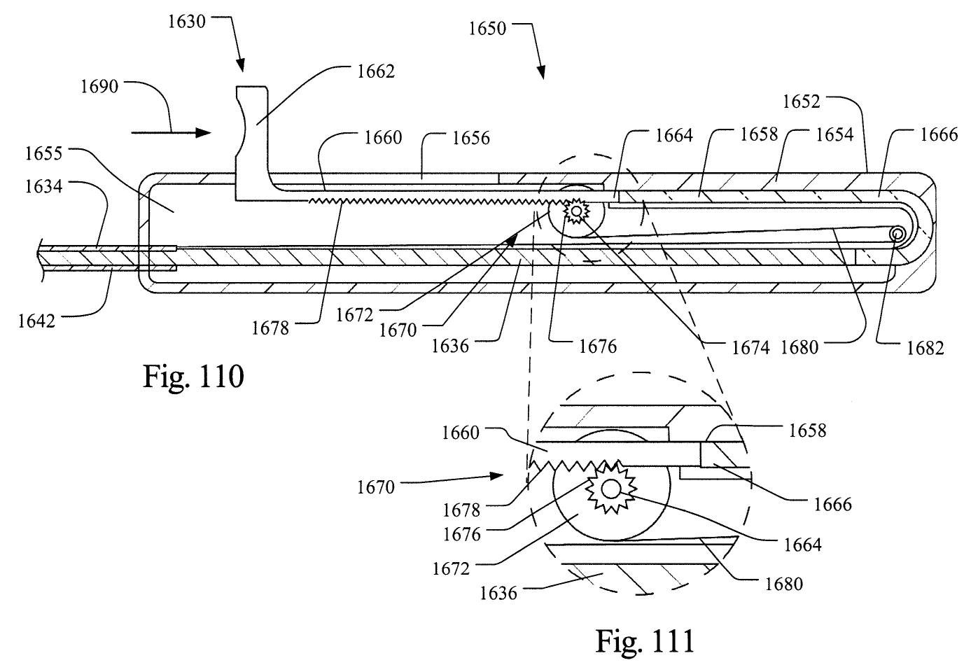

Alternatively, delivery system 1630 may include an actuator 1650 having a handle 1652 operatively coupled to inner sheath 1634 and outer sheath 1642. Handle 1652 may include a housing 1654 defining a chamber 1655 and a slot 1656 along at least a portion of a length of housing 1654. Further, as shown in FIG. 110, housing 1654 defines an inner passage 1658. A retraction element 1660 is positioned within slot 1656. Retraction element 1660 may include a first portion 1662 external to housing 1654 and a second portion 1664 at least partially positioned within inner passage 1658. In one example, a semi-rigid or bendable member 1666 is at least partially positioned within inner passage 1658 between second portion 1664 and support member 1636. A pulley/spindle assembly 1670 is rotatably positioned within housing 1654 and may include a pulley 1672 and a spindle 1674 coaxially coupled to pulley 1672. Spindle 1674 forms a plurality of teeth 1676 that cooperate with a plurality of corresponding teeth 1678 formed on retraction element 1660, as described in greater detail below. Pulley 1672 is coupled to inner sheath 1634 with a string 1680. String 1680 is coupled at a first end to pulley 1672 and is positioned about a pulley 1682. A second end of string 1680 is coupled to inner sheath 1634.

Referring to FIGS. 110 and 111, retraction element 1660 is moved in a distal direction as shown by directional arrow 1690 in FIG. 110. As retraction element 1660 is moved, teeth 1678 cooperate with teeth 1676 of spindle 1674 to rotate pulley/spindle assembly 1670. As pulley 1672 rotates, string 1680 is wrapped about an outer periphery of pulley 1670 to retract inner sheath 1634. Simultaneously, retraction element 1660 pushes semi-rigid member 1666 through inner passage 1658 to contact support member 1636. Semi-rigid member 1666 urges support member 1636 to advance in the proximal direction to maintain support stent 126 properly positioned at the lesion site and with respect to graft 114.

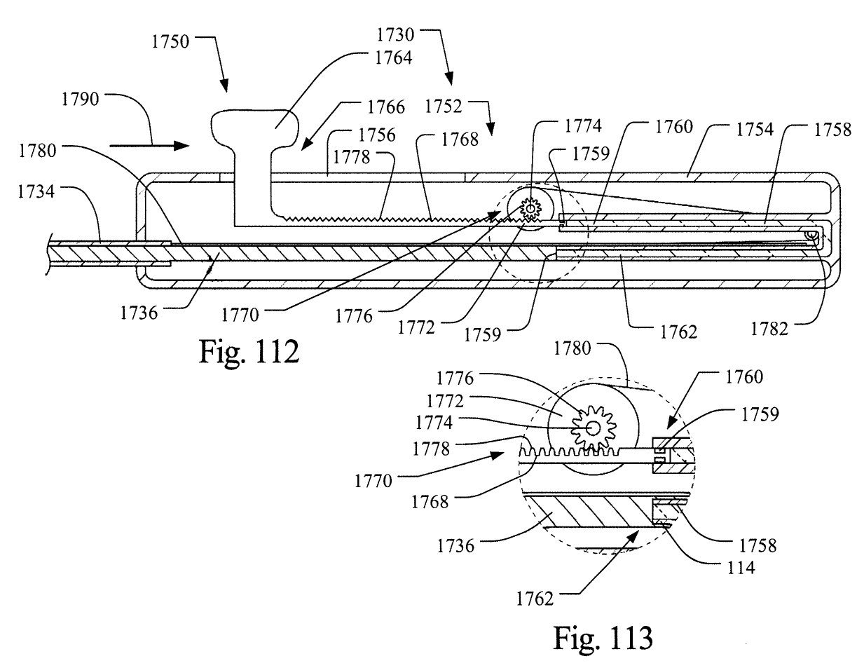

Alternatively, delivery system 1730 may include an actuator 1750 having a handle 1752 operatively coupled to inner sheath 1734 and an outer sheath (not shown). Handle 1752 may include a housing 1754 defining a chamber 1755 and a slot 1756 along at least a portion of a length of housing 1754. Further, as shown in FIG. 112, housing 1754 defines an inner passage 1758. Inner passage 1758 may include a sealing member 1759, such as an O-ring or other suitable sealing member, positioned at an inlet end 1760 and a generally opposing outlet end 1762 and configured to sealingly contain a hydraulic fluid, such as water, within inner passage 1758. A retraction element 1764 is positioned within slot 1756. Retraction element 1764 may include a first portion 1766 external to housing 1754 and a second portion 1768 at least partially positioned within inner passage 1758. A pulley/spindle assembly 1770 is rotatably positioned within housing 1754 and includes a pulley 1772 and a spindle 1774 coaxially coupled to pulley 1772. Spindle 1774 forms a plurality of teeth 1776 that cooperate with a plurality of corresponding teeth 1778 formed on second portion 1768 of retraction element 1764, as described in greater detail below. Pulley 1772 is coupled to inner sheath 1734 with a string 1780. String 1780 is coupled at a first end to pulley 1772 and is positioned about a pulley 1782. A second end of string 1780 is coupled to inner sheath 1734.

Referring to FIGS. 112 and 113, retraction element 1764 is moved in a distal direction as shown by directional arrow 1790 in FIG. 112. As retraction element 1764 is moved, teeth 1778 cooperate with teeth 1776 of spindle 1774 to rotate pulley/spindle assembly 1770. As pulley 1772 rotates, string 1780 is wrapped about an outer periphery of pulley 1772 to retract inner sheath 1734. Simultaneously, retraction element 1764 provides a force against the hydraulic fluid within inner passage 1758 to advance support member 1736. The hydraulic fluid urges support member 1736 to advance in the proximal direction to maintain support stent 126 properly positioned at the lesion site and with respect to graft 114.

Delivery System for Generic Prosthesis

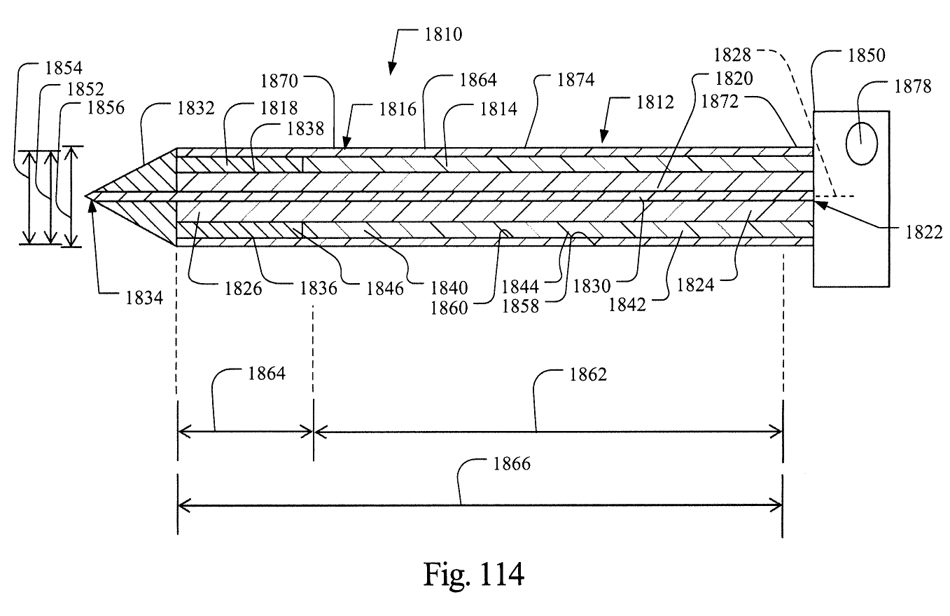

FIG. 114 is a partial sectional view of a delivery system 1810. Components of delivery system 1810 may have any suitable shape, size and/or configuration. Delivery system 1810 can be used in conjunction with a plurality of components including, without limitation, a balloon catheter, a dual balloon catheter a trans-medicinal catheter and/or a multi-branched catheter.

In one example, prosthesis delivery system 1810 may include a catheter 1812 including a support member 1814 and a catheter sheath 1816. Delivery system 1810 also may include an expandable balloon (not shown). A prosthesis 1818, such as a stent or stent graft, is positioned on delivery system 1810.

Catheter 1812 has any suitable shape and/or size. Further, catheter 1812 is fabricated using any suitable material that enables catheter 1812 to function as described herein. Catheter 1812 may include an elongate shaft 1820 defining a guide wire passage 1822 extending therethrough from a proximal end 1824 to a distal end 1826 along an axis 1828.

In operation, a guide wire 1830 extends through guide wire passage 1822 to guide delivery system 1810 to a target location or lesion site, as shown in FIG. 94-A. In one example, a nose cone 1832 is coupled to shaft distal end 1826. Nose cone 1832 may include a guide wire passage 1834 extending therethrough. Nose cone 1832 facilitates advancement of catheter 1812 through a body lumen to the lesion site.

Shaft 1820 may be slidably coupled to support member 1814 and/or prosthesis 1818. Specifically, at least a portion of shaft 1820, such as distal end 1826, is circumferentially surrounded by support member 1814 and prosthesis 1818. Alternatively, shaft distal end 1826 is coupled to an expandable balloon (not shown) which extends within prosthesis 1818.

Prosthesis 1818 may be a tubular, radially expandable prosthesis, such as a stent, a vascular graft, a stent graft composite, a nitinol stent, a covered stent, a mesh stent, a braided stent, a tapered stent, a Z stent, a Wallstent or a combination thereof. Prosthesis 1818 may include any suitable prosthesis. In this example, prosthesis 1818 is radially expandable between a generally unexpanded configuration having an unexpanded delivery diameter and an expanded or configuration having an expanded or deployment diameter, which is greater than the delivery diameter. Prosthesis 1818 is flexible and coupled to shaft 1820 in a radially compressed configuration and then expanded at the lesion site. In one example, prosthesis 1818 is fabricated from self-expandable material having a spring-like action and/or memory properties, such as temperature-dependant memory properties. Alternatively, a balloon positioned with respect to prosthesis 1818 facilitates expansion of prosthesis 1818. Prosthesis 1818 is radially distensible or deformable.

Prosthesis 1818 may have any suitable geometry and/or configuration. Further, prosthesis 1818 may be fabricated of any suitable biocompatible material including, without limitation, a suitable metal, such as stainless steel, platinum, gold and titanium, an alloy and/or a polymeric material. In one example, prosthesis 1818 is fabricated from a Nitinol material, which exhibits a spring-like or shape-memory deformation.

In one example, prosthesis 1818 may include an outer surface 1836 in frictional contact with sheath 1816 and an inner surface 1838 in frictional contact with shaft 1820. Prosthesis 1818 is positioned between support member 1814 and nose cone 1832. Prosthesis 1818 is configured to be deployed by support member 1814 and/or sheath 1816.

Support member 1814 defines a distal end 1840 and an opposing proximal end 1842. An elongate body 1844 extends between distal end 1840 and proximal end 1842. In one example, body 1844 was a tubular shape forming a passage through which shaft 1820 extends. In alternative example, body 1844 has any suitable shape and/or size. In one example, support member 1814 is fabricated from Pebax. Alternatively, support member 1814 is fabricated from a suitable polymeric material, such as a polyether amide, or any suitable material that enables support member 1814 to function as described herein.

Support member distal end 1840 may be positioned adjacent a prosthesis proximal end 1846 and in a contacting relationship with proximal end 1846. Specifically, support member 1814 is releasably coupled to prosthesis 1818. In one example, support member proximal end 1842 is coupled to a catheter handle 1850, which will be discussed in greater detail below.

Support member body 1844 has a diameter 1852 substantially equal to an unexpanded diameter 1854 of prosthesis 1818 and less than an inner diameter 1856 of sheath 1816. Support member 1814 is sized to fit within sheath 1816 and slidably contact an inner surface 1858 of sheath 1816. Support member 1814 and sheath 1816 are fabricated with tight tolerances such that a frictional force exists between sheath inner surface 1858 and a support member outer surface 1860. Specifically, support member 1814 frictionally contacts sheath 1816, and is movable within sheath 1816. As will be discussed in further detail below, support member 1814 is configured to contact and/or engage and deploy prosthesis 1818 at the lesion site.

Support member 1814 has a suitable length 1862. In one example, length 1862 is greater than a prosthesis length 1864 and less than a sheath length 1866. Lengths 1862, 1864, 1866, and diameters 1852, 1854, 1856, may have different lengths and/or diameters than the above-indicated lengths and/or diameters, depending upon the particular application.

Catheter sheath 1816 defines a distal end 1870, and an opposing proximal end 1872. An elongate body 1874 extends between distal end 1870 and proximal end 1872. Body 1874 defines a housing, a sleeve, a sock or any suitable assembly for surrounding and retaining prosthesis 1818 and/or support member 1814 properly position on catheter 1812. In one example, body 1874 has a tubular shape. Sheath 1816 is sized to overlay prosthesis 1818 and support member 1814. Body 1874 has any suitable shape and/or size. Sheath 1816 may be substantially shorter than support member 1814. In one example, sheath 1816 is retractable. Sheath 1816 may be coupled to handle 1850 and is configured to move in a proximal direction and/or distal direction.

In one example, sheath 1816 is fabricated from a braided, reinforced extruded material. Alternatively, sheath 1816 is fabricated from Pebax material or any suitable polymeric material. Sheath 1816 may be fabricated from a suitable material that enables sheath 1816 to function as described herein.

In one example, sheath 1816 is configured to have a yield strength greater than a self-expansion force of prosthesis 1818. As such, sheath 1816 retains prosthesis 1818 in a compressed or unexpanded configuration during delivery of prosthesis 1818. While the yield strength of sheath 1816 is sufficient to maintain prosthesis 1818 in a compressed state, sheath 1816 is configured to axially move over an outside surface 1876 of support member 1814 along axis 1828 during deployment. In one example, sheath 1816 is slidably coupled with prosthesis 1818 and/or support member 1814 for facilitating retaining of prosthesis 1818 adjacent and/or in contacting relationship with support member 1814 during delivery and deployment of prosthesis 1818. In one example, sheath 1816 is releasably coupled to nose cone 1832.

Handle 1850 is configured to simultaneously impart relative movement to support member 1814 and sheath 1816 in opposite directions. More specifically, handle 1850 simultaneously imparts a proximal movement on support member 1814 and a distal movement on sheath 1816 during deployment of prosthesis 1818. This relative movement is in an axial direction and the ratio of movement is based, at least partially, on a predetermined foreshortening percentage of prosthesis 1818. In one example, this relative movement ratio is based on the specific prosthesis included in delivery system 1810. Handle 1850 may include an adjustable relative movement control member 1878 configured to vary the amount of axial force according to the predetermined foreshortening percentage of prosthesis 1818 and the specific usage of delivery system 1810.

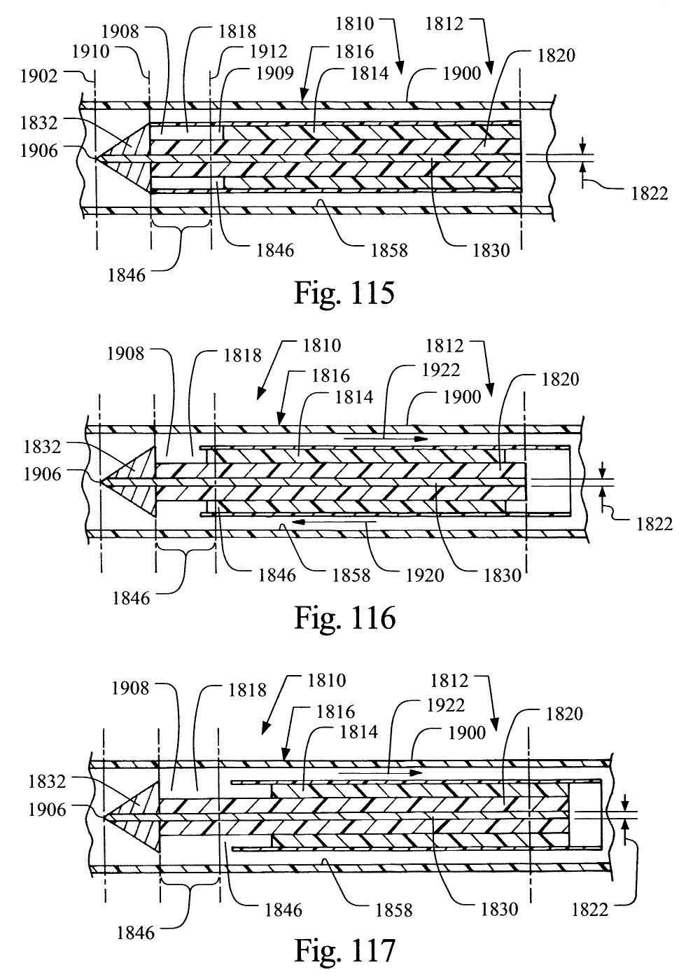

FIG. 115 is a sectional view of an exemplary prosthesis delivery system 1810 before deployment. FIG. 116 is a sectional view of an exemplary prosthesis delivery system 1810 during deployment. FIG. 117 is a sectional view of an exemplary prosthesis delivery system 1810 after deployment. FIGS. 115-117 share common location reference numbers to aid in understanding the deployment of delivery system 1810 at selected stages of deployment. These numbers are for illustration and are not meant to limit in any way the application of prosthesis delivery system 1810.

In one example, prosthesis 1818 is a self-expanding stent 1819 configured to contact and/or engage an interior surface of lumen wall 1900. Before deployment, stent 1819 is releasably coupled to or loaded on shaft 1820 in a compressed configuration. Guide wire 1830 is percutaneously inserted into a patient's lumen or vessel, and guide wire 1830 is guided to a location 1902 proximal to a target location or lesion site 1904 such that guide wire distal end 1906 is positioned at lesion site 1904. Catheter 1812 is then positioned such that guide wire 1830 extends through passage 1822 in nose cone 1832 and shaft 1820. Nose cone 1832 is guided to lesion site 1904 such that stent proximal end 1908 is positioned at a target location proximal end 1910 and stent distal end 1909 is positioned at a target location distal end 1912.

During deployment at lesion site 1904, support member 1814 advances proximal while, simultaneously, sheath 1816 retracts distally and guide wire end 1906 and nose cone 1832 are kept stationary relative to location 1902. More specifically, a first axial force is applied to support member 1814 in a proximal direction 1920 along axis 1828. The first axial force is greater than the frictional force applied against sheath inner surface 1858 by compressed stent 1819 and support member 1814, thus support member 1814 engages stent 1819. Simultaneously, a second axial force is applied in a distal direction 1922 opposite proximal direction 1920 and sheath 1816 releases stent 1819 which begins to expand as stent 1819 exits sheath 1816. The second axial force is greater that the frictional force applied by prosthesis 1818 and/or the interior surface of lumen wall 1900. In this example, “simultaneously” refers to the first and second axial forces imparted substantially concurrently.

The amount of the first axial force is sufficient to maintain stent 1819 stationary. In one example, first axial force and second axial force are determined by the foreshortening percentage of stent 1819 as well as the friction between sheath 1816 and stent 1819 and/or support member 1814. In one example, the first axial force and the second axial force are equal. In another example, the first axial force and the second axial force are different.

After deployment of stent 1819, stent 1819 is fully expanded and accurately positioned at lesion site 1904. Specifically, stent proximal end 1908 is positioned at target location proximal end 1910 and stent distal end 1909 is positioned at target location distal end 1912. Additionally, guide wire end 1906 remains at location 1902. Catheter 1812 including guide wire 1830, nose cone 1832, support member 1814, sheath 1816 and shaft 1820 are withdrawn in distal direction 1922 from the patient, leaving stent 1819 properly positioned.

While FIGS. 115-177 illustrate a delivery system to facilitate accurate positioning of a self-expanding prosthesis, the advantages apply to all types of prostheses. The system can be sized and configured for use in various body lumens, specifically, any other lumen where accurate location of a stent or prosthesis is desired.

While the invention has been described in terms of various specific embodiments, those skilled in the art will recognize that the invention can be practiced with modification within the spirit and scope of the claims.

1. A delivery system for deploying a stent graft in a body vessel, comprising:
a wire lumen;
a support stent slidably positioned about the wire lumen and having a proximal end and a distal end, the support stent expandable from a compressed delivery configuration to an expanded configuration;
an inner sheath retractably positioned about the support stent with the support stent in the delivery configuration;
an anchor stent slidably positioned about the inner sheath and having a proximal end and a distal end, the anchor stent deployable from a compressed delivery configuration to a deployed configuration,
a tubular graft having a proximal end and a distal end, the graft proximal end coupled to the anchor stent and deployable with the anchor stent from a compressed delivery configuration to a deployed configuration; and
an outer sheath retractably positioned about the anchor stent and the graft in the compressed delivery configuration.

2. The delivery system of claim 1 further comprising a support member positioned about the wire lumen, the support member having a proximal end and a distal end, where the proximal end of the support member contacts the distal end of the support stent in the delivery configuration.

3. The delivery system of claim 2 where the support member is configured to substantially maintain a position of the support stent in the body vessel as the inner sheath is retracted from about the support stent.

4. The delivery system of claim 1 further comprising an actuator operatively coupled to the anchor stent, the actuator configured to retract the outer sheath and deploy the anchor stent, and retract the inner sheath and deploy the support stent.

5. The delivery system of claim 4 further comprising:
a housing;
an outer sheath retraction tube coupled to the outer sheath slideably positioned within the housing and movable in a distal direction to retract the outer sheath and deploy the anchor stent;
a first locking element configured to lock the outer sheath retraction tube in a locked position and limit movement of the outer sheath retraction tube within the housing;
an inner sheath retraction tube slidably positioned about the outer sheath retraction tube and coupling the inner sheath to the first locking element; and
a second locking element configured to lock the inner sheath retraction tube in a locked position and limit movement of the inner sheath retraction tube relative to the outer sheath retraction tube;
where the first locking element is movable relative to the outer sheath in a distal direction to retract the inner sheath and deploy the support stent.

10. A delivery system for deploying a stent graft in a body vessel, comprising:
a wire lumen slidably positionable about a guide wire;
a support stent having a proximal end and a distal end slidably positioned about the wire lumen and expandable from a compressed delivery configuration to an expanded configuration;
an inner sheath retractably positioned about the support stent with the support stent in the compressed delivery configuration;
an anchor stent having a proximal end and a distal end slidably positioned about the inner sheath and deployable from a compressed delivery configuration to a deployed configuration;
an outer sheath retractably positioned about the anchor stent with the anchor stent in the insertion configuration; and
a handle operatively coupled to each of the inner sheath and the outer sheath.

11. The delivery system of claim 10 where the handle comprises:
a housing;
an outer sheath retraction tube coupled to the outer sheath and slideably positioned within the housing and movable in a distal direction to retract the outer sheath and deploy the anchor stent;
a first locking element configured to lock the outer sheath retraction tube in a locked position and limit movement of the outer sheath retraction tube within the housing;
an inner sheath retraction tube slidably positioned about the outer sheath retraction tube and coupling the inner sheath to the first locking element; and
a second locking element configured to lock the inner sheath retraction tube in a locked position and limit movement of the inner sheath retraction tube relative to the outer sheath retraction tube;
where the first locking element is movable relative to the outer sheath in a distal direction to retract the inner sheath and deploy the support stent.

12. The delivery system of claim 11 where each of the outer sheath retraction tube and the inner sheath retraction tube has an anti-rotational cross-sectional area.

13. The delivery system of claim 10 where the handle comprises:
a housing defining an axis and a track along at least a portion of the axis, the track including a plurality of locking grooves;
a locking element positioned about the housing and operatively coupled to the outer sheath, where, in a locked position the locking element is positioned within a first locking groove of the plurality of locking grooves to limit movement of the outer sheath and, where, in an unlocked position the first locking element is slidably movable with respect to the housing in a distal direction along the track to retract the outer sheath;
a second locking element positioned about the housing and operatively coupled to the anchor stent, where, in a locked position the second locking element positioned within a second locking groove of the plurality of locking grooves to limit movement of the anchor stent and, where, in an unlocked position the second locking element is slidably movable with respect to the housing in the distal direction along the track to deploy the anchor stent; and
a third locking element positioned about the housing and operatively coupled to the inner sheath, where, in a locked position the third locking element is positioned within a third locking groove of the plurality of locking grooves to limit movement of the inner sheath and, where, in an unlocked position the third locking element is slidably movable with respect to the housing in the distal direction along the track to retract the inner sheath and deploy the support stent.

14. The delivery system of claim 10 where the handle comprises:
a housing defining a track along at least a portion of a length of the housing;
a retraction element operatively coupled to the outer sheath, the retraction element rotatable with respect to the housing between a locked position and an unlocked position, where in the unlocked position the retraction element is slidably movable with respect to the housing in a distal direction between an initial position and a first stop position to retract the outer sheath and between the first stop position and a second stop position to retract the inner sheath.

15. The delivery system of claim 14 further comprising a first connector coupling the retraction element to the outer sheath, the first connector slidably positioned within the track.

16. The delivery system of claim 15 further comprising a second connector coupled to the inner sheath, the second connector at least partially positioned within the track and configured to interfere with the first connector as the retraction element is moved from the first stop position to the second stop position.

17. The delivery system of claim 11 where the handle comprises:
a housing defining a chamber along at least a portion of a length of the housing, at least a portion of the outer sheath and at least a portion of the inner sheath movable within the chamber;
a button coupled to the housing and operatively coupled to the outer sheath to retain the outer sheath in an insertion configuration;
a biasing element positioned within the chamber and coupled to a distal end of the housing, the biasing element biasing the outer sheath towards the distal end; and
a retracting element operatively coupled to the inner sheath and rotatable with respect to the housing between a locked position and an unlocked position, where in the unlocked position the retraction element is slidably movable with respect to the housing in a distal direction to retract the inner sheath; and
a second biasing element positioned within the chamber and coupled to the distal end of the housing, the second biasing element biasing the inner sheath towards the distal end.

18. A delivery system for deploying an endoluminal prosthesis within a body lumen at a target location, comprising:
a delivery sheath having a proximal end and a distal end and configured to retain the prosthesis within the delivery system in an unexpanded configuration at the delivery sheath distal end;
a support member having a proximal end and a distal end positioned at least partially within the delivery sheath, where the proximal end of the support member is adjacent to the distal end of the prosthesis; and
a handle configured to impart relative movement to at least one of the delivery sheath and the support member.

19. The delivery system of claim 18 where the handle is configured to simultaneously axially retract the delivery sheath in a distal direction and axially advance the support member in a proximal direction, such that the support member engages the distal end of the prosthesis and maintains the prosthesis at the target location.

20. The delivery system of claim 19, where the prosthesis is releasable during deployment from the delivery sheath along a longitudinal axis by an axial force from the support member, and where the axial force is greater than a frictional force between the delivery sheath and the prosthesis.

21. The delivery system of claim 20, where the support member is configured to move a distance within the delivery sheath, the distance determined by a foreshortening percentage of the prosthesis.

22. A delivery system comprising:
a shaft defining a guide wire passage;
a support member having a proximal end and a distal end movably coupled to the shaft, and configured to advance in a proximal direction along the shaft; and
a tubular delivery sheath configured to at least partially surround the support member and to retract in a distal direction along the shaft.

23. The delivery system of claim 22, further comprising a radially expandable prosthesis having a proximal end and a distal end, and where the support member is positioned within the delivery sheath and adjacent the distal end of the prosthesis.

24. The delivery system of claim 23 further comprising a handle configured to impart relative axial movement to the delivery sheath in a distal direction and to the support member in an opposing proximal direction substantially simultaneously, where the prosthesis decreases in length upon radial expansion, and where the support member contacts the prosthesis and maintains the proximal end of the prosthesis at a target location.

25. A delivery system for deploying a stent graft in a body vessel, comprising:
a wire lumen;
a support stent slidably positioned about the wire lumen and having a proximal end and a distal end, the support stent expandable from a compressed delivery configuration to an expanded configuration;
an inner sheath retractably positioned about the support stent with the support stent in the compressed delivery configuration;
an anchor stent slidably positioned about the inner sheath and having a proximal end and a distal end, the anchor stent deployable from a compressed insertion configuration to a deployed configuration,
a tubular graft having a proximal end and a distal end, the graft proximal end coupled to the anchor stent and deployable with the anchor stent from a compressed delivery configuration to a deployed configuration; and
an outer sheath retractably positioned about the anchor stent and the graft in the compressed delivery configuration; and
a capture mechanism operatively coupled to the proximal end of the anchor stent, the capture mechanism initially configured to retain the proximal end of the stent in a delivery configuration, the capture mechanism actuatable to release the proximal end of the anchor stent.
